(19) United States
(12) Patent Application Publication
(10) Pub. No.: US 2026/0055120 A1
(43) Pub. Date: Feb. 26, 2026

(54) Bicyclic pyrimidinones as inhibitors of NOX4

(71) Applicant: Boehringer Ingelheim International GmbH, Ingelheim am Rhein (DE)

(72) Inventors: Christian GNAMM, Biberach an der Riss (DE); Elmar BAUSCHATZ, Schemmerhofen (DE); Frank H. BUETTNER, Attenweiler (DE); Cédrickx GODBOUT, Schemmerhofen (DE); Annekatrin Charlotte HEIMANN, Biberach an der Riss (DE); Christoph HOENKE, Biberach an der Riss (DE); Ferenc KONTES, Biberach (DE); Christian Andreas KUTTRUFF, Donaueschingen (DE); Dieter WIEDENMAYER, Biberach an der Riss (DE); Raphael WILDERMUTH, Ingelheim am Rhein (DE)

(21) Appl. No.: 19/308,754

(22) Filed: Aug. 25, 2025

(30) Foreign Application Priority Data
Aug. 26, 2024 (EP) 24196346.1

(51) Int. Cl. C07D 513/04; A61K 31/519; A61K 45/06; C07D 495/04

(52) U.S. Cl. CPC C07D 513/04; A61K 31/519; A61K 45/06; C07D 495/04

(57) ABSTRACT

The present disclosure provides bicyclic pyrimidinone derivatives that are inhibitors of NOX4 and therefore useful for the treatment of diseases treatable by inhibition of NOX4. Also provided are pharmaceutical compositions containing the same, and processes for preparing said compounds.

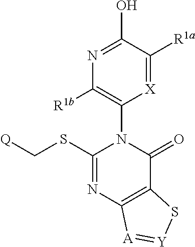

### TECHNICAL FIELD

The present disclosure provides certain thieno- and thiazolo-pyrimidinone derivatives that are inhibitors of NOX4, and are therefore useful for the treatment of diseases treatable by inhibition of NOX4. Furthermore, the invention relates to pharmaceutical compositions and combinations comprising these compounds, as well as their use in methods for the treatment of diseases associated with or modulated by NOX4. Particularly, the pharmaceutical compositions of the invention are suitable for the therapy of interstitial lung diseases, e.g. idiopathic pulmonary disease, and may also be suitable for the therapy of fibrotic diseases, allergic and inflammatory diseases.

### BACKGROUND INFORMATION

Redox signaling is a critical part of a variety of cell signaling pathways involved in the regulation of cell growth, differentiation, metabolism, immune regulation, and other physiological functions. It is characterized by an oxidation-reduction reaction or covalent adduct formation between the sensor and the second messenger. Thereby oxidation of the sensor protein can modulate its activity, conformation, or susceptibility towards degradation. In case of redox signaling the second messengers consist of a group of reactive oxygen and nitrogen species (ROS, RNS). These are generated as side products during metabolic reactions in the mitochondria but also by specialized enzymes like NADPH-oxidases (NOXes). In parallel, a complex antioxidant system evolved to protect the cells from injury by ROS and RNS, ensuring a tight balance between ROS/RNS formation and degradation. In case this balance is disturbed, oxidative stress can occur leading to increased protein oxidation and aberrant redox signaling. Therefore, oxidative stress is associated with various pathophysiological conditions like interstitial lung diseases, cancer and inflammatory diseases.

Several studies showed over the last decade that in interstitial lung diseases, like idiopathic pulmonary fibrosis (IPF), the redox balance is disturbed¹ and that NOX4 is specifically upregulated in the lungs of IPF patients¹. IPF patients experience a decline in their lung function and blood oxygenation, which is caused by the remodeling of the lung parenchyma in combination with the stiffening of the lung tissue due to increased matrix deposition. Upon epithelial injury due to e.g. infection, air pollutants etc., fibroblasts are activated to enable migration of immune cells as well as epithelial precursor cells to the site of injury. Once the injury is resolved fibroblasts as well as immune cells undergo apoptosis to allow complete restoration of the tissue. In IPF an aberrant repair is occurring leading to a progressive remodeling of the lung parenchyma². Recent studies using single cell sequencing allowed the identification and comparison of different cell types of lung tissue derived from IPF patients or healthy controls. These studies showed increased numbers of fibroblasts as well as a strong dedifferentiation of the epithelial cell population³.

NOX4 is part of the NADPH oxidase enzyme family. NOXes are membrane bound multisubunit protein complexes, which transfer electrons across the plasma membrane to generate ROS. While the activity of all other family members (NOX1, NOX2, NOX3 and NOX5) is tightly controlled and inducible, NOX4 is the only family member that is constitutively active¹. It is localized in the plasma membrane, perinuclear vesicles, the endoplasmatic reticulum (ER), the mitochondria and the nuclear membrane. The generation of hydrogen peroxide (H₂O₂) by NOX4 leads to reversible oxidation of cysteine thiol groups and/or S-glutathionylation¹. Thereby, NOX4 is modulating TGFβ signaling and other key pathways known to be involved in disease progression¹.

IPF fibroblasts show higher expression of NOX4 in comparison to fibroblasts isolated from healthy tissue, as well as hyperplastic alveolar cells. The role of NOX4 in fibroblast activation as well as proliferation was investigated in lung fibroblasts¹. Fibroblast proliferation, as well as fibroblast activation are known processes driving disease progression. Immunohistochemistry on control and IPF lung samples confirmed increased NOX4 staining in fibroblasts and showed stronger staining in bronchial and alveolar epithelial cells¹.

Acute respiratory distress syndrom (ARDS) and acute lung injury (ALI) are characterized by pulmonary infiltration and edema. Literature shows that NOX4 is increased in patients and plays a crucial role in cell recruitment in animal models⁴.

Metabolic Dysfunction-associated Steatohepatitis (MASH) is characterized by an increase in lipid content and inflammation in the liver. The chronic feedback loop of lipotoxicity, increased inflammation and cell death leads to liver fibrosis. Throughout that process liver cells, namely stellate cells, dedifferentiate to an activated phenotype, of which one hallmark is the induction of pro-fibrotic and pro-inflammatory expression pattern. As on main redoxmediating signalling pathway, the NOX-family—especially NOX4—plays a crucial role in that feedback loop. NOX4-dependent signalling directly leads to an induction of NFkB and MAPk transcription leading to induction of gene products, such as smooth muscle actin (SMA), collagen or tumor necrosis factor (TNF).

Published literature shows that NOX4 is expressed in stellate cells and is expressed through the process of stellate cell activation. Vice versa, the inhibition of NOX4 on the other hand leads to attenuated expression of stellate cell-activation markers.

In addition to in vitro analyses, studies in NOX4 knock-out mice show reduced fibrosis and inflammation in MASH-relevant models.

Aside from the prevention of fibrosis onset and progression, NOX4 plays a crucial role of endothelial integrity. Thereby, NOX4 activity fosters the nitric oxide (NO) production, leading to vascular relaxation and reduced endothelial inflammation. Following, NOX4 inhibition has a beneficial impact on portal hypertension.

In cancer, ROS deregulation contributes to tumor development, progression, and metastasis.

In some instances, this enhanced ROS, driven by various oncogenic perturbations, is required for tumorigenicity leading to the acquisition of further DNA damage and genome instability in cancer cells⁵. Increased ROS occurs through a variety of mechanisms such as increased expression of the NOX proteins or NOX activators, or the downregulation of ROS-regulating systems. NOX4 itself has been reported to be increased in many types of tumors leading to increased proliferation, migration, and apoptosis⁵. Tumor associated macrophages are influenced by ROS and can adopt an immunosuppressive phenotype within tumors in response to ROS⁵. Furthermore, when fibroblasts are educated to become cancer associated fibroblasts (CAFs), NOX4 is upregulated and leads to CD8+ T cell exclusion and immune suppression in tumors. Deletion or inhibition of NOX4 in several mouse models of cancer, has reversed this immune suppression and restored immunotherapy response to anti-PD-1 therapy (PD1 being programmed cell death protein 1)⁵. Therefore, specific inhibition of NOX proteins in tumor cells themselves or stromal cells, is a tractable target for anti-tumor therapies.

The list of the references cited above can be found here:
1. Veith, C., Boots, A. W., Idris, M., Schooten, F.-J. van & Vliet, A. van der. Redox Imbalance in Idiopathic Pulmonary Fibrosis: A Role for Oxidant Cross-Talk Between NADPH Oxidase Enzymes and Mitochondria. Antioxid Redox Sign 31, 1092-1115 (2019).
2. Hinz, B. & Lagares, D. Evasion of apoptosis by myofibroblasts: a hallmark of fibrotic diseases. Nat Rev Rheumatol 16, 11-31 (2020).
3. Habermann, A. C. et al. Single-cell RNA sequencing reveals profibrotic roles of distinct epithelial and mesenchymal lineages in pulmonary fibrosis. Sci Adv 6, eaba1972 (2020).
4. Lee, S. H. et al. NADPH oxidase 4 signaling in a ventilator-induced lung injury mouse model. Respir Res 23, 73 (2022).
5. Cheung, E. C. & Vousden, K. H. The role of ROS in tumour development and progression. Nat Rev Cancer 22, 280-297 (2022).

### DETAILED DESCRIPTION OF THE INVENTION

The present invention discloses novel thieno- and thiazolo-pyrimidinone derivatives that are inhibitors of NOX4, possessing appropriate pharmacological and pharmacokinetic properties enabling their use as medicaments for the treatment of conditions and/or diseases treatable by inhibition of NOX4.

PCT/EP2024/054867 (i.e. WO2024/180018) discloses thiazolo-pyrimidinone derivatives as NOX4-inhibitors and particularly discloses a thiazole-pyrimidinone substituted with a 2-hydroxy-pyrimidine as example 69.

WO2016207785 discloses benzoxazole and benzthiazole based NOX4 inhibitors with the following generalized structural formula

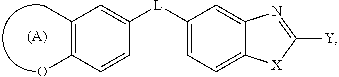

of which example 1, example 15 and example 80 are reported with the following IC₅₀ values in an assay on modified human embryonic kidney (HEK) cells using Amplex Red as detecting reagent:

| Example as dis- | | NOX4 IC₅₀ [μM] |
|---|---|---|
| closed in | | as disclosed in |
| WO2016207785 | Structure | WO2016207785 |
| 1 | 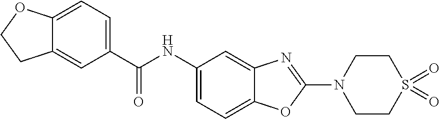 [SMILES: O=C(Nc1ccc2oc(N3CCS(=O)(=O)CC3)nc2c1)c1ccc2c(c1)CCO2] | 0.34 |
| 15 | 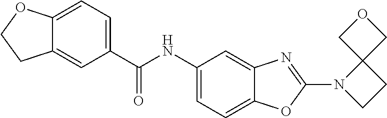 [SMILES: O=C(Nc1ccc2oc(N3CCC34COC4)nc2c1)c1ccc2c(c1)CCO2] | 0.40 |
| 80 | 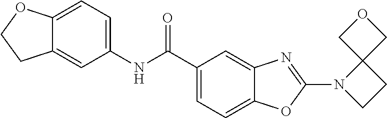 [SMILES: O=C(Nc1ccc2c(c1)CCO2)c1ccc2oc(N3CCC34COC4)nc2c1] | 0.31 |

When tested in an assay for metabolic stability as described herein below, examples 1, 15 and 80 show a clearance in human hepatocytes of 52% QH, 29% QH and 46% QH, respectively.

Another patent application, WO2005049613 discloses bicyclic pyrimidin-4-(3H)-one based modulators of the vanilloid-1 receptor (VR1) of the following generalized structural formula

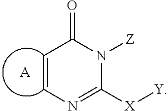

Examples 19, 20, 30, 59 and 63 from WO2005049613 have been tested for their activity on NOX4 inhibition with the assay described herein below, resulting in the following IC₅₀ values:

| Example as | | |
|---|---|---|
| disclosed in | | NOX4 |
| WO2005049613 | Structure | IC₅₀ [μM] |
| 19 | 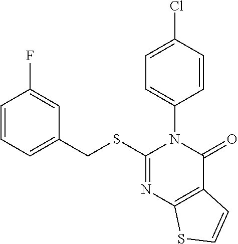 [SMILES: O=c1c2ccsc2nc(SCc2cccc(F)c2)n1-c1ccc(Cl)cc1] | >99 |
| 20 | 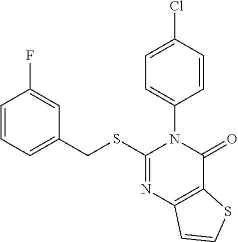 [SMILES: O=c1c2sccc2nc(SCc2cccc(F)c2)n1-c1ccc(Cl)cc1] | >99 |
| 30 | 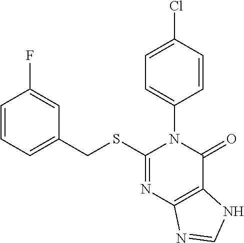 [SMILES: O=c1c2[nH]cnc2nc(SCc2cccc(F)c2)n1-c1ccc(Cl)cc1] | >99 |
| 59 | 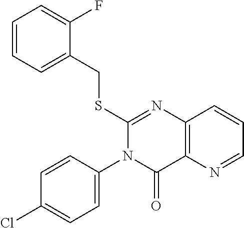 [SMILES: O=c1c2ncccc2nc(SCc2ccccc2F)n1-c1ccc(Cl)cc1] | 79 |
| 63 | 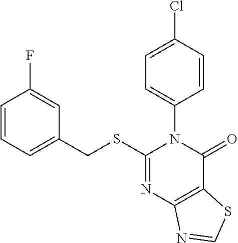 [SMILES: O=c1c2scnc2nc(SCc2cccc(F)c2)n1-c1ccc(Cl)cc1] | 55 |

As visible from the above data, the tested compounds of WO2005049613 show low activity as inhibitors of NOX4.

Surprisingly it was found that the exchange of the para-chloro substituent in example 63 from WO2005049613 with a para-hydroxy substituent increases the inhibition of NOX4 from 55 μM to 0.66 μM with respect to IC₅₀ as tested in the NOX4-inhibition assay described herein below, as shown by the following reference compound and the following examples 3 and 57 of the present invention in the following table:

| | | NOX4 |
|---|---|---|
| Example | Structure | IC₅₀ [μM] |
| Reference Example | 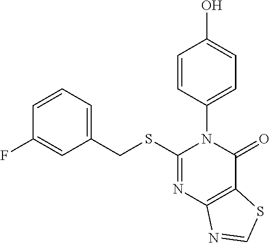 [SMILES: O=c1c2scnc2nc(SCc2cccc(F)c2)n1-c1ccc(O)cc1] | 0.56 |
| Example 3 of the present invention | 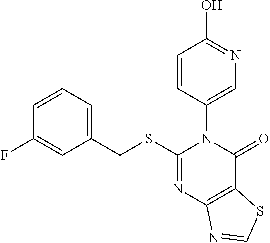 [SMILES: O=c1c2scnc2nc(SCc2cccc(F)c2)n1-c1ccc(O)nc1] | 0.84 |
| Example 57 of the present invention | 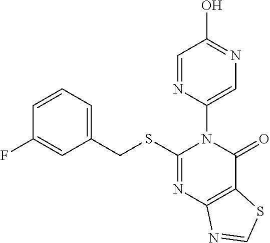 [SMILES: O=c1c2scnc2nc(SCc2cccc(F)c2)n1-c1cnc(O)cn1] | 0.10 |

The effect of the hydroxy group in this position on the NOX4 inhibitory activity of other compounds of the present invention is also shown in the following table:

| Example | Structure | NOX4 IC₅₀ [μM] |
|---|---|---|
| Reference Example | 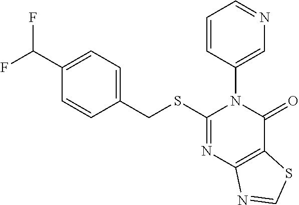 [SMILES: O=c1c2scnc2nc(SCc2ccc(C(F)F)cc2)n1-c1cccnc1] | 8.47 |
| Example 4 of the present invention | 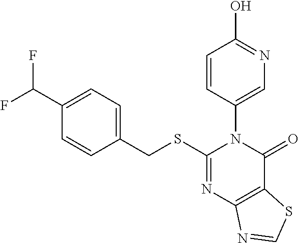 [SMILES: O=c1c2scnc2nc(SCc2ccc(C(F)F)cc2)n1-c1ccc(O)nc1] | 0.24 |
| Example 61 of the present invention | 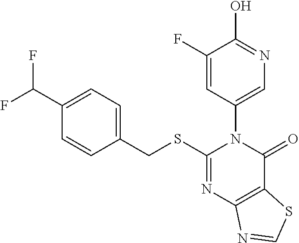 [SMILES: O=c1c2scnc2nc(SCc2ccc(C(F)F)cc2)n1-c1cnc(O)c(F)c1] | 0.064 |
| Reference Example | 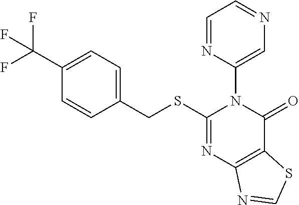 [SMILES: O=c1c2scnc2nc(SCc2ccc(C(F)(F)F)cc2)n1-c1cnccn1] | 3.81 |
| Example 6 of the present invention | 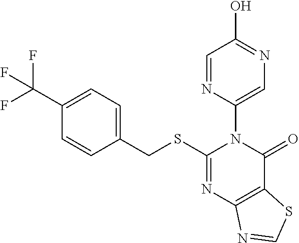 [SMILES: O=c1c2scnc2nc(SCc2ccc(C(F)(F)F)cc2)n1-c1cnc(O)cn1] | 0.036 |

It is therefore the aim of the present invention to provide thiazolo- and thieno-pyrimidinones which are potent, metabolically stable and selective NOX4 inhibitors. Such thiazolo- and thieno-pyrimidinones of the invention may also be represented as thiazolo- and thieno-2-hydroxypyrimidines.

The compounds of the present invention according to general formula (I)

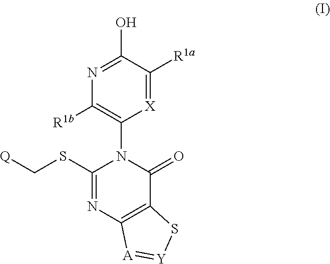

wherein
A is N or CH;
X is N or CR^{1c};
Y is N or CH;
R^{1a}, R^{1b} and R^{1c} is H or F;
Q is selected from among a group consisting of

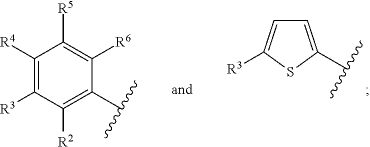

R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R⁷ is —Cl or —CF₃; or salts thereof, exhibit several advantageous properties such as high potency, high metabolic stability and high selectivity for NOX4 and against NOX1, NOX2, NOX3 and NOX5.

The compounds according to the invention typically show inhibition of NOX4 with IC₅₀-values below 600 nM, preferably below 400 nM, more preferably below 200 nM, most preferably below 100 nM (see assay description herein below and table 1). High potency can enable lower doses for pharmacological efficacy. Lower doses have the advantages of lower “drug load” or “drug burden” (parent drug and metabolites thereof) for the patient causing potentially less side effects, and lower production costs for the drug product.

In a further aspect of the invention, the compounds according to the invention are selective NOX4-inhibitors, and are selective against NOX1, NOX2, NOX3 and NOX5. Preferred are compounds in which the IC₅₀ on NOX4 is 10× lower than the IC₅₀ on either of NOX1, NOX2, NOX3 or NOX5. More preferred are compounds in which the IC₅₀ on NOX4 is 30× lower than the IC₅₀ on either of NOX1, NOX2, NOX3 or NOX5. Most preferred are compounds in which the IC₅₀ on NOX4 is 100× lower than the IC₅₀ on either of NOX1, NOX2, NOX3 or NOX5 (see assay description herein below and table 2).

Furthermore, the compounds according to the invention are metabolically stable as shown in human hepatocytes. Metabolic stability in human hepatocytes in this respect is defined as below or equal to 45% QH, preferably below or equal to 30% QH, more preferably below or equal to 20% QH (see assay description herein below and table 3 and the definition of how to calculate the % QH=hepatic blood flow herein below). Therefore, the compounds of the present invention are expected to have a favorable in vivo clearance and thus the favourably long duration of action in humans. Stability in human hepatocytes refers to the susceptibility of compounds to biotransformation in the context of selecting and/or designing drugs with favorable pharmacokinetic properties, as the primary site of metabolism for many drugs is the liver. Human hepatocytes contain the cytochrome P450 (CYPs) and additional enzymes for phase II metabolism (e.g. phosphatases and sulfatases), and thus represent a model system for studying in vitro how a drug is metabolised. Stability in hepatocytes is associated with several advantages, including improved bioavailability and half-life, which can allow lower and less frequent dosing in patients. Thus, stability in hepatocytes is a favorable characteristic for compounds that are to be used as drugs in the treatment of a disease.

### USED TERMS AND DEFINITIONS

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached. In case a chemical bond at the end of moiety is indicated with a “-”, this “-” signals the attachment point of the group to the molecule.

In case a compound of the present invention is depicted in the form of a chemical name and as a formula, in case of any discrepancy the formula shall prevail. A wavy line may be used in sub-formulas to indicate the bond which is connected to the core molecule as defined. For example, the term “3-carboxypropyl-group” represents the following substituent:

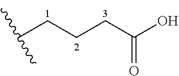 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

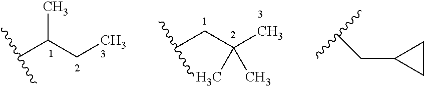

The wavy line may be used in sub-formulas to indicate the bond which is connected to the core molecule as defined.

### Term Substituted

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like. Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

### Salts

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

### Halogen

The term halogen denotes fluorine, chlorine, bromine and iodine.

### Alkyl

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

### Alkylene

The term “C₁₋ₙ-alkylene” wherein n is an integer selected from 2, 3, 4, 5 or 6, preferably 4, 5 or 6, either alone or in combination with another radical, denotes an acyclic, saturated, branched or linear chain divalent alkyl radical containing from 1 to n carbon atoms. For example the term C₁₋₄-alkylene includes —CH₂—, —CH₂—CH₂—, —CH(CH₃)—, —CH₂—CH₂—CH₂—, —C(CH₃)₂—, —CH(CH₂CH₃)—, —CH(CH₃)—CH₂—, —CH₂—CH(CH₃)—, —CH₂—CH₂—CH₂—CH₂—, —CH₂—CH₂—CH(CH₃)—, —CH(CH₃)—CH₂—CH₂—, —CH₂—CH(CH₃)—CH₂—, —CH₂—C(CH₃)₂—, —C(CH₃)₂—CH₂—, —CH(CH₃)—CH(CH₃)—, —CH₂—CH(CH₂CH₃)—, —CH(CH₂CH₃)—CH₂—, —CH(CH₂CH₂CH₃)—, —CH(CH(CH₃))₂— and —C(CH₃)(CH₂CH₃)—.

### Alkenyl

The term “C₂₋ₘ-alkenyl” is used for a group “C₂₋ₘ-alkyl” wherein m is an integer selected from 3, 4, 5 or 6, preferably 4, 5 or 6, if at least two carbon atoms of said group are bonded to each other by a double bond.

### Alkenylene

The term “C₂₋ₘ-alkenylene” is used for a group “C₂₋ₘ-alkylene”, wherein m is an integer selected from 3, 4, 5 or 6, preferably 4, 5 or 6, if at least two carbon atoms of said group are bonded to each other by a double bond.

### Alkynyl

The term “C₂₋ₘ-alkynyl” is used for a group “C₂₋ₘ-alkyl” wherein m is an integer selected from 3, 4, 5 or 6, preferably 4, 5 or 6, if at least two carbon atoms of said group are bonded to each other by a triple bond.

### Alynylene

The term “C₂₋ₘ-alkynylene” is used for a group “C₂₋ₘ-alkylene” wherein m is an integer selected from 3, 4, 5 or 6, preferably 4, 5 or 6, if at least two of those carbon atoms of said group are bonded to each other by a triple bond.

### Cycloalkyl

The term “C₃-k-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

Halo-(alkyl, alkylene or cycloalkyl)

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: F₃C—, H₂FC—, HF₂C.

Many of the terms given above may be used repeatedly in the definition of a formula or group and in each case have one of the meanings given above, independently of one another.

The term “bicyclic ring systems” means groups consisting of 2 joined cyclic substructures including spirocyclic, fused, and bridged ring systems.

### PREFERRED EMBODIMENTS

In another embodiment the invention relates to compounds of general formula (Ia)

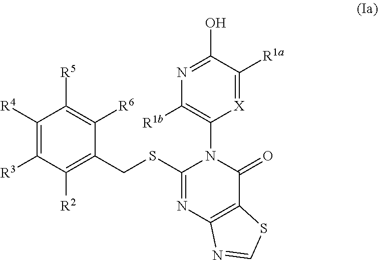

wherein X is N or C—R^{1c}, wherein R^{1a}, R^{1b} and R^{1c} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ia) wherein X is N or C—R^{1c}, wherein R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ia) wherein X is N or C—R^{1c}, wherein R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ia) wherein X is N or C—R^{1c}, wherein R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ia) wherein X is N or C—R^{1c}, wherein R^{1c} is H; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ia) wherein X is N or C—R^{1c}, wherein R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Ia) wherein X is N or C—R^{1c}, wherein R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Ia) wherein X is N or C—R^{1c}, wherein R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (Ia) wherein X is N or C—R^{1c}, wherein R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ia) wherein X is N or C—R^{1c}, wherein R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ia) wherein X is N and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ia) wherein X is N and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ia) wherein X is N and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Ia) wherein X is N and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂Cl and —OCH₃—CN, —SF₅ and —N₃; R³ is H and R⁵ is H; or a salt thereof.

In another embodiment the invention relates to compounds of general formula (Ia) wherein X is N and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (Ia) wherein X is CR^{1c} and R^{1c} is H and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ia) wherein X is CR^{1c} and R^{1c} is H and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ia) wherein X is CR^{1c} and R^{1c} is H and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ia) wherein X is CR^{1c} and R^{1c} is H and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Ia) wherein X is CR^{1c} and R^{1c} is H and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Ia) wherein X is CR^{1c} and R^{1c} is H and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (I)

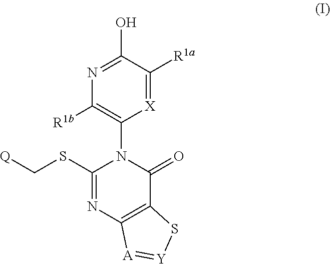

wherein Q is selected from among a group consisting of

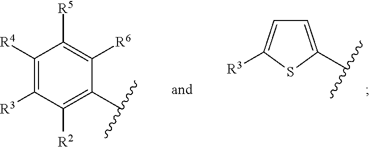

wherein A is N, X is N, and Y is CH; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; and wherein R⁷ is —Cl or —CF₃; or a salt thereof.

In another embodiment the invention relates to compounds of general formula (I) wherein Q is selected from among a group consisting of

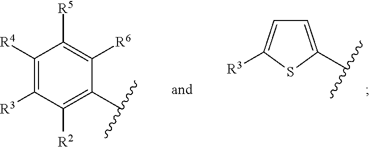

wherein A is N, X is N, and Y is CH; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; and wherein R⁷ is —Cl or —CF₃; or a salt thereof.

In another embodiment the invention relates to compounds of general formula (I) wherein Q is selected from among a group consisting of

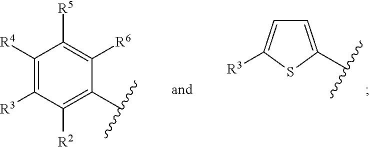

A is N, X is N, and Y is CH; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; and wherein R⁷ is —Cl or —CF₃; or a salt thereof.

In another embodiment the invention relates to compounds of general formula (I) wherein Q is selected from among a group consisting of

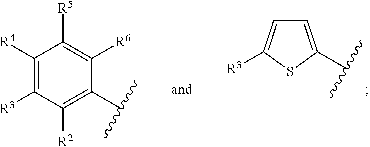

A is N, X is N, and Y is CH; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; and wherein R⁷ is —Cl or —CF₃; or a salt thereof.

In another embodiment the invention relates to compounds of general formula (I) Q is selected from among a group consisting of

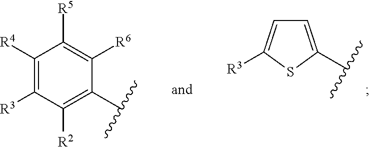

A is N, X is N, and Y is CH; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; and wherein R⁷ is —Cl or —CF₃; or a salt thereof.

In another embodiment the invention relates to compounds of general formula (I) wherein Q is selected from among a group consisting of

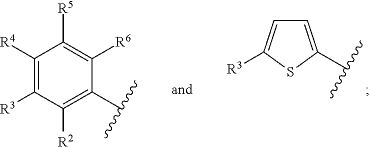

A is N, X is N, and Y is CH; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; and wherein R⁷ is —Cl or —CF₃; or a salt thereof.

In another embodiment the invention relates to compounds of general formula (I)

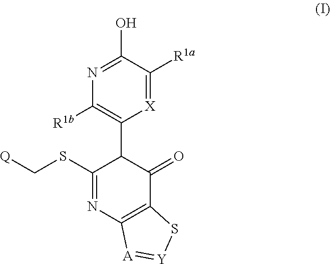

wherein Q is selected from among a group consisting of

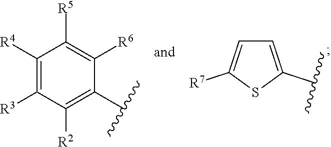

wherein A is N, X is CR^{1c}, R^{1c} is H and Y is CH; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; and wherein R⁷ is —Cl or —CF₃; or a salt thereof.

In another embodiment the invention relates to compounds of general formula (I) wherein Q is selected from among a group consisting of

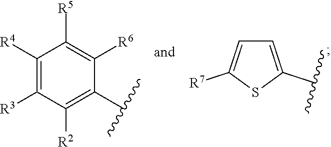

A is N, X is CR^{1c}, R^{1c} is H and Y is CH; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; and wherein R⁷ is —Cl or —CF₃; or a salt thereof.

In another embodiment the invention relates to compounds of general formula (I) wherein Q is selected from among a group consisting of

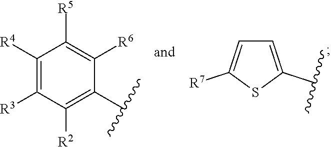

A is N, X is CR^{1c}, R^{1c} is H and Y is CH; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; and wherein R⁷ is —Cl or —CF₃; or a salt thereof.

In another embodiment the invention relates to compounds of general formula (I) wherein Q is selected from among a group consisting of

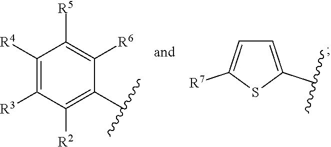

A is N, X is CR^{1c}, R^{1c} is H and Y is CH; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; and wherein R⁷ is —Cl or —CF₃; or a salt thereof.

In another embodiment the invention relates to compounds of general formula (I) wherein Q is selected from among a group consisting of

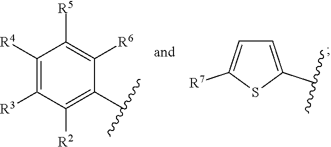

A is N, X is CR^{1c}, R^{1c} is H and Y is CH; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; and wherein R⁷ is —Cl or —CF₃; or a salt thereof.

In another embodiment the invention relates to compounds of general formula (I) wherein Q is selected from among a group consisting of

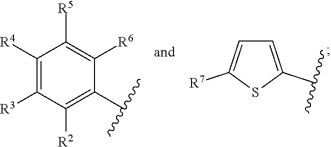

A is N, X is CR^{1c}, R^{1c} is H and Y is CH; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; and wherein R⁷ is —Cl or —CF₃; or a salt thereof.

In another embodiment the invention relates to compounds of general formula (Ib)

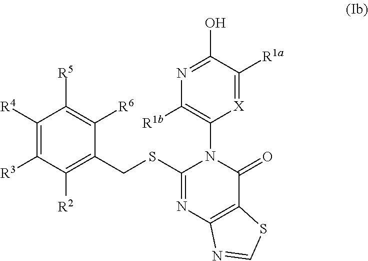

wherein X is N or CR^{1c}; and wherein R^{1a}, R^{1b} and R^{1c} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ib) wherein X is N or CR^{1c}; and wherein R^{1a} and R^{1b} is H or F and R^{1c} is H; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ib) wherein X is N or CR^{1c}; and wherein R^{1a} and R^{1b} is H or F and R^{1c} is H; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ib) wherein X is N or CR^{1c}; and wherein R^{1a} and R^{1b} is H or F and R^{1c} is H, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ib) wherein X is N or CR^{1c}; and wherein R^{1a} and R^{1b} is H or F and R^{1c} is H, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Ib) wherein X is N or CR^{1c}; and wherein R^{1a} and R^{1b} is H or F and R^{1c} is H, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Ib) wherein X is N or CR^{1c}; and wherein R^{1a} and R^{1b} is H or F and R^{1c} is H, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (Ib) wherein X is N; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ib) wherein X is N; and wherein R^{1a} and R^{1b} are, independently of each other, H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof; with the proviso that such compounds of general formula (Ib) are not compounds of general formula (Ib) wherein X is N; R^{1a} and R^{1b} are H; R², R⁴, and R⁶ are, independently of each other, —H or —F; and R³ and R⁵ are H.

In another embodiment the invention relates to compounds of general formula (Ib) wherein X is N; and wherein R^{1a} and R^{1b} are, independently of each other, H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof; with the proviso that the compound is not

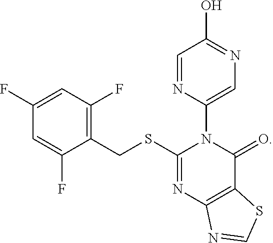 [SMILES: O=c1c2scnc2nc(SCc2c(F)cc(F)cc2F)n1-c1cnc(O)cn1]

In another embodiment the invention relates to compounds of general formula (Ib) wherein X is N; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ib) wherein X is N; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ib) wherein X is N; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general (Ib) wherein X is N; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Ib) wherein X is N; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (Ib) wherein X is N; and wherein R^{1a} and R^{1b} are, independently of each other, H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof; with the proviso that such compounds of general formula (Ib) are not compounds of general formula (Ib) wherein X is N; R^{1a} and R^{1b} are H; R², R⁴, and R⁶ are, independently of each other, —H or —F; and R³ and R⁵ are H.

In another embodiment the invention relates to compounds of general formula (Ib) wherein X is N; and wherein R^{1a} and R^{1b} are, independently of each other, H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof; with the proviso that the compound is not

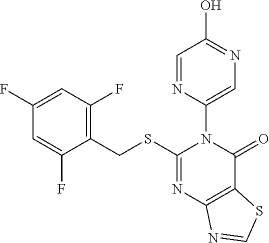 [SMILES: O=c1c2scnc2nc(SCc2c(F)cc(F)cc2F)n1-c1cnc(O)cn1]

(disclosed in WO2024/180018).

In another embodiment the invention relates to compounds of general formula (Ib) wherein X is CR^{1c}, R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Ib) wherein X is CR^{1c}, R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ib) wherein X is CR^{1c}, R^{1c} is H; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Ib) wherein X is CR^{1c}, R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Ib) wherein X is CR^{1c}, R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Ib) wherein X is CR^{1c}, R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (Ic)

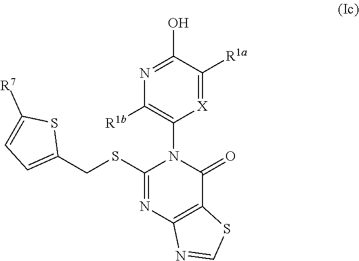

wherein R^{1a} and R^{1b} is H or F; and wherein X is N or C—R^{1c}; and wherein R^{1c} is H; and wherein R⁷ is —Cl or —CF₃; or a salt thereof.

In another embodiment the invention relates to compounds of general formula (Ic) wherein R^{1a} and R^{1b} is H or F, provided that R^{1a} and R^{1b} cannot be F simultaneously; and wherein X is N or C—R^{1c}; and wherein R^{1c} is H; and wherein R⁷ is —Cl or —CF₃; or a salt thereof.

In another embodiment the invention relates to compounds of general formula (Ic) wherein X is N; and wherein R^{1a} and R^{1b} is H or F; and wherein R⁷ is —Cl or —CF₃; or a salt thereof.

In another embodiment the invention relates to compounds of general formula (Ic) wherein X is N; and wherein R^{1a} and R^{1b} is H or F, provided that R^{1a} and R^{1b} cannot be F simultaneously; and wherein R⁷ is —Cl or —CF₃; or a salt thereof.

In another embodiment the invention relates to compounds of general formula (Ic) wherein X is CR^{1c}, R^{1c} is H; and wherein R^{1a} and R^{1b} is H or F; and wherein R⁷ is —Cl or —CF₃; or a salt thereof.

In another embodiment the invention relates to compounds of general formula (Ic) wherein X is CR^{1c}, R^{1c} is H; and wherein R^{1a} and R^{1b} is H or F, provided that R^{1a} and R^{1b} cannot be F simultaneously; and wherein R⁷ is —Cl or —CF₃; or a salt thereof.

In another embodiment the invention relates to compounds of general formula (Id)

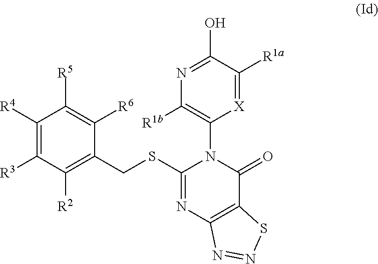

wherein X is N or CR^{1c}; and wherein R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, -cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Id) wherein X is N or CR^{1c}; and wherein R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, -cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Id) wherein X is N or CR^{1c}; and wherein R^{1c} is H; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, -cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Id) wherein X is N or CR^{1c}; and wherein R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, -cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Id) wherein X is N or CR^{1c}; and wherein R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, -cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Id) formula (Id) wherein X is N or CR^{1c}; and wherein R^{1c} is H and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, -cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (Id) wherein X is N; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Id) wherein X is N; and wherein R^{1a} and R^{1b} is H or F; and wherein R², R³, R⁴, R⁵, R⁶ are, independently of each other, selected from among a group consisting of —H, -halogen, —C₁₋₄-alkyl, -cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Id) wherein X is N; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, -cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Id) wherein X is N; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, -cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Id) wherein X is N; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, -cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Id) wherein X is N; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, -cyclopropyl, —CH═CH₂, —C≡CH, —CF₃, —CF₂H, —CF₂Cl and —OCH₃; R³ is H and R⁵ is H; or a salt thereof.

In another embodiment the invention relates to compounds of general formula (Id) wherein X is N; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, -cyclopropyl, —CF₃, and —CF₂H; R³ is H and R⁵ is H; or a salt thereof.

In another embodiment the invention relates to compounds of general formula (Id) wherein X is CR^{1c} and R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

In another embodiment the invention relates to compounds of general formula (Id) wherein X is CR^{1c} and R^{1c} is H; and wherein R^{1a} and R^{1b} is H or F; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Id) wherein X is CR^{1c} and R^{1c} is H; and wherein R^{1a} and R^{1b} is H or F, provided that R^{1a} and R^{1b} cannot be F simultaneously; and wherein R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, R⁶ are H; or a salt thereof.

In another embodiment the invention relates to compounds of general formula (Id) wherein X is CR^{1c} and R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; R³ is H and R⁵ is H; or a salt thereof.

In another embodiment the invention relates to compounds of general formula (Id) wherein X is CR^{1c} and R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —Br, —I, —C₁₋₂-alkyl, cyclopropyl, —CH═CH₂, —C≡CH, —CF₃, —CF₂H, —CF₂C₁, —OCH₃, —CN, —SF₅ and —N₃; R³ is H and R⁵ is H; or a salt thereof.

In another embodiment the invention relates to compounds of general formula (Id) wherein X is CR^{1c} and R^{1c} is H; and wherein R^{1a} and R^{1b} is H or F, provided that R^{1a} and R^{1b} cannot be F simultaneously; and wherein R², R⁴, R⁶ are, independently of each other, selected from among a group consisting of —H, —F, —Cl, —C₁₋₂-alkyl, cyclopropyl, —CH═CH₂, —C≡CH, —CF₃, —CF₂H, —CF₂Cl and —OCH₃; R³ is H and R⁵ is H; or a salt thereof.

In another embodiment, the invention relates to a compound selected from among the group consisting of

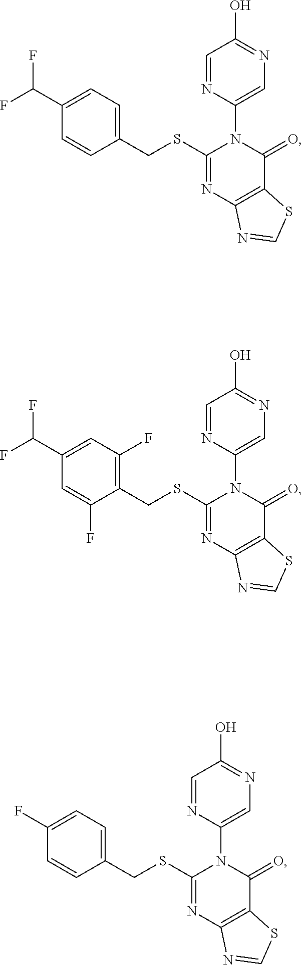 [SMILES: O=c1c2scnc2nc(SCc2ccc(C(F)F)cc2)n1-c1cnc(O)cn1 ; O=c1c2scnc2nc(SCc2c(F)cc(C(F)F)cc2F)n1-c1cnc(O)cn1 ; O=c1c2scnc2nc(SCc2ccc(F)cc2)n1-c1cnc(O)cn1]

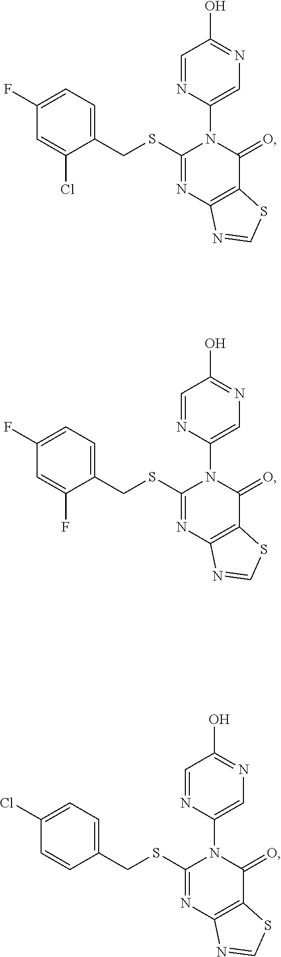 [SMILES: O=c1c2scnc2nc(SCc2ccc(F)cc2Cl)n1-c1cnc(O)cn1 ; O=c1c2scnc2nc(SCc2ccc(F)cc2F)n1-c1cnc(O)cn1 ; O=c1c2scnc2nc(SCc2ccc(Cl)cc2)n1-c1cnc(O)cn1]

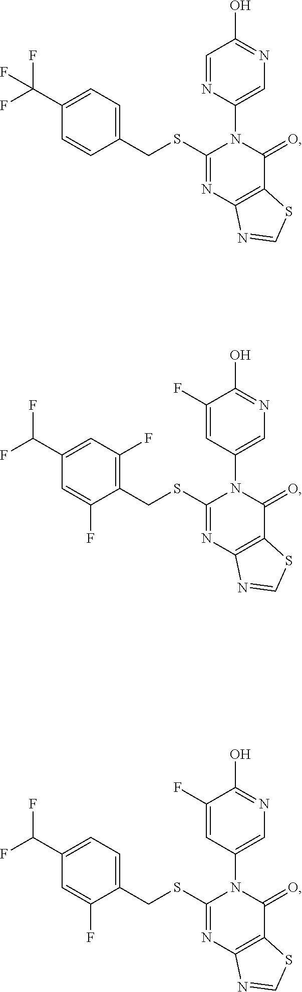 [SMILES: O=c1c2scnc2nc(SCc2ccc(C(F)(F)F)cc2)n1-c1cnc(O)cn1 ; O=c1c2scnc2nc(SCc2c(F)cc(C(F)F)cc2F)n1-c1cnc(O)c(F)c1 ; O=c1c2scnc2nc(SCc2ccc(C(F)F)cc2F)n1-c1cnc(O)c(F)c1]

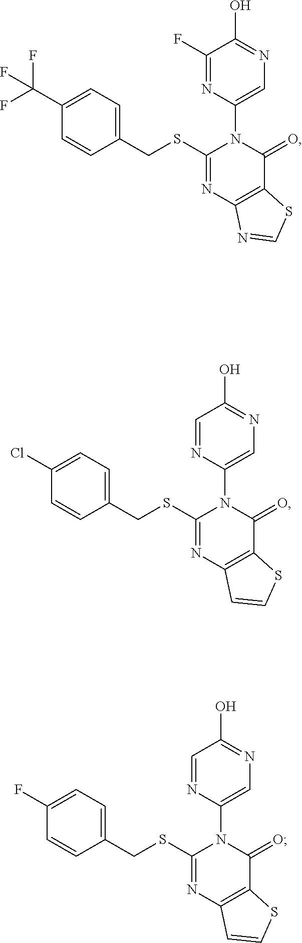 [SMILES: O=c1c2scnc2nc(SCc2ccc(C(F)(F)F)cc2)n1-c1cnc(O)c(F)n1 ; O=c1c2sccc2nc(SCc2ccc(Cl)cc2)n1-c1cnc(O)cn1 ; O=c1c2sccc2nc(SCc2ccc(F)cc2)n1-c1cnc(O)cn1]

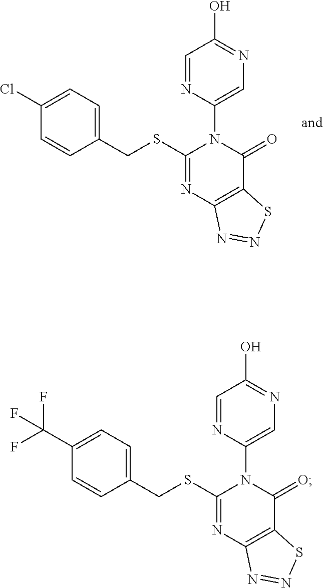 [SMILES: O=c1c2snnc2nc(SCc2ccc(Cl)cc2)n1-c1cnc(O)cn1 ; O=c1c2snnc2nc(SCc2ccc(C(F)(F)F)cc2)n1-c1cnc(O)cn1]

in its salt-free form.

In another embodiment, the invention relates to a compound selected from among the group consisting of

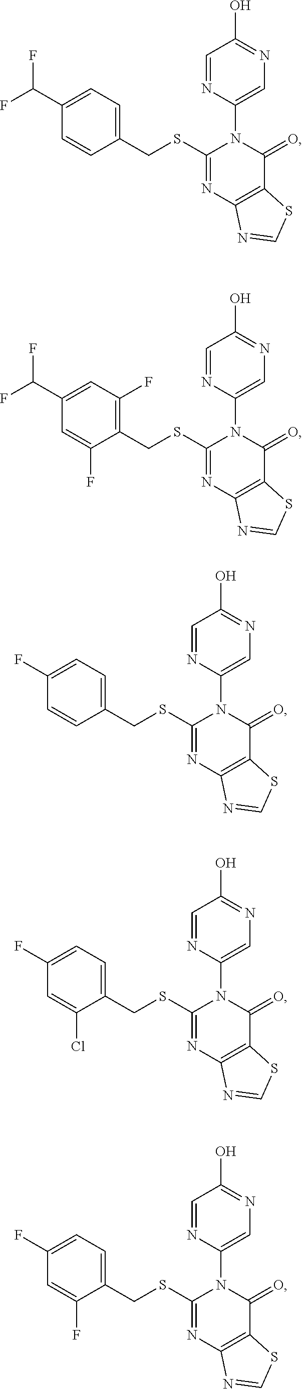 [SMILES: O=c1c2scnc2nc(SCc2ccc(C(F)F)cc2)n1-c1cnc(O)cn1 ; O=c1c2scnc2nc(SCc2c(F)cc(C(F)F)cc2F)n1-c1cnc(O)cn1 ; O=c1c2scnc2nc(SCc2ccc(F)cc2)n1-c1cnc(O)cn1 ; O=c1c2scnc2nc(SCc2ccc(F)cc2Cl)n1-c1cnc(O)cn1 ; O=c1c2scnc2nc(SCc2ccc(F)cc2F)n1-c1cnc(O)cn1]

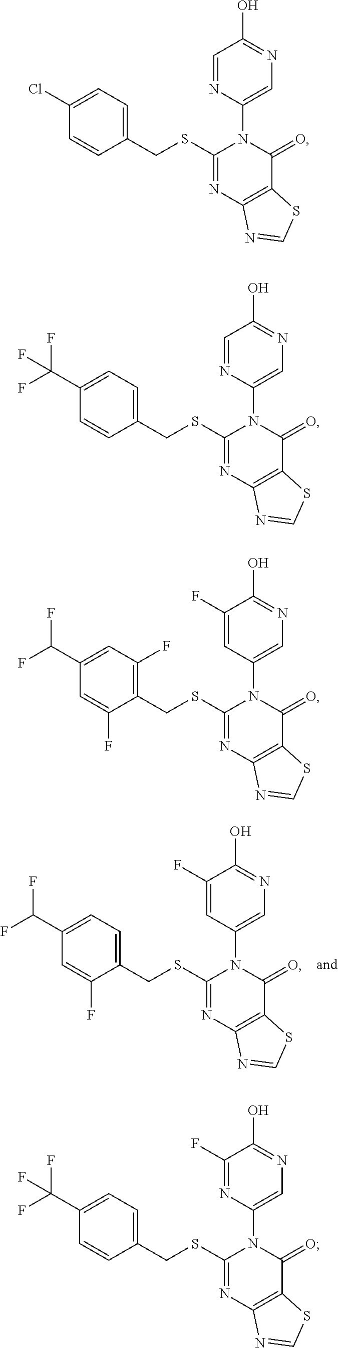

or a pharmaceutically acceptable salt thereof.

In another embodiment, the invention relates to a compound selected from among the group consisting of

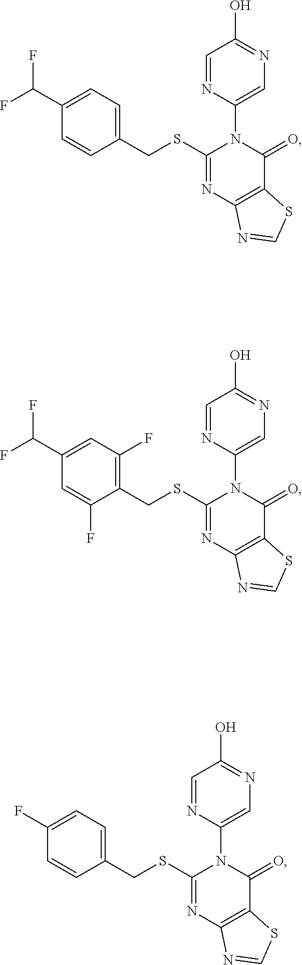 [SMILES: O=c1c2scnc2nc(SCc2ccc(C(F)F)cc2)n1-c1cnc(O)cn1 ; O=c1c2scnc2nc(SCc2c(F)cc(C(F)F)cc2F)n1-c1cnc(O)cn1 ; O=c1c2scnc2nc(SCc2ccc(F)cc2)n1-c1cnc(O)cn1]

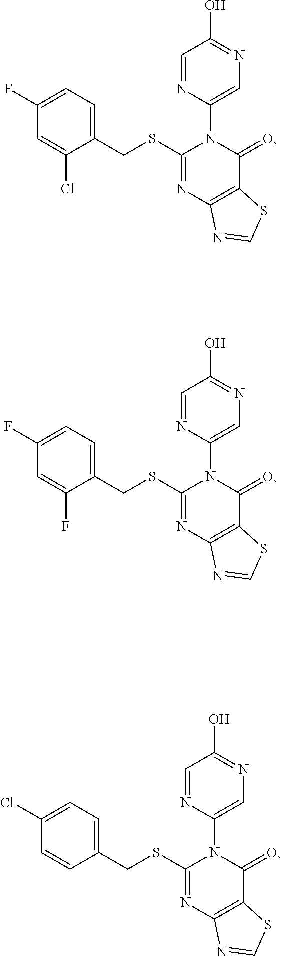 [SMILES: O=c1c2scnc2nc(SCc2ccc(F)cc2Cl)n1-c1cnc(O)cn1 ; O=c1c2scnc2nc(SCc2ccc(F)cc2F)n1-c1cnc(O)cn1 ; O=c1c2scnc2nc(SCc2ccc(Cl)cc2)n1-c1cnc(O)cn1]

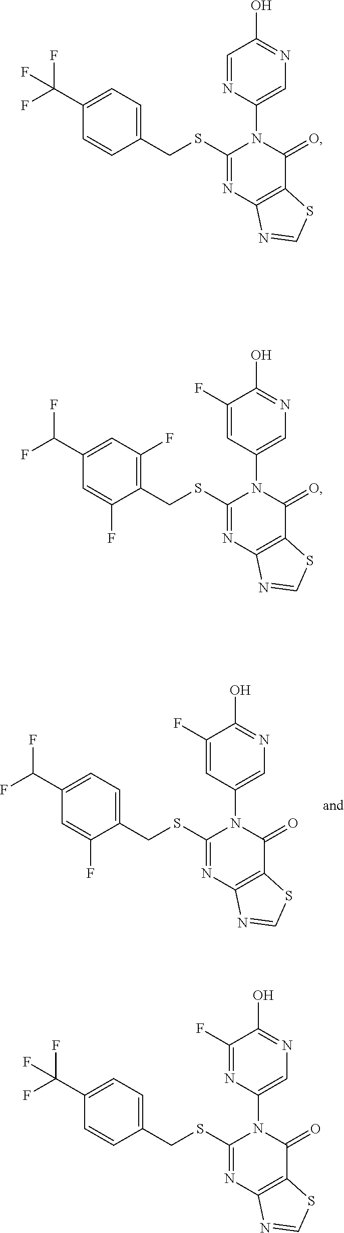 [SMILES: O=c1c2scnc2nc(SCc2ccc(C(F)(F)F)cc2)n1-c1cnc(O)cn1 ; O=c1c2scnc2nc(SCc2c(F)cc(C(F)F)cc2F)n1-c1cnc(O)c(F)c1 ; O=c1c2scnc2nc(SCc2ccc(C(F)F)cc2F)n1-c1cnc(O)c(F)c1 ; O=c1c2scnc2nc(SCc2ccc(C(F)(F)F)cc2)n1-c1cnc(O)c(F)n1]

in its salt-free form.

In another embodiment, the invention relates to a compound selected from the group consisting of

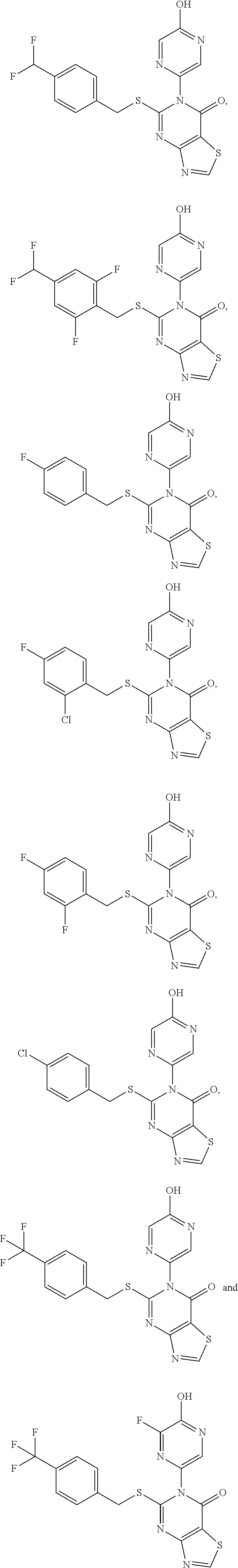

in its salt-free form or a salt thereof or a pharmaceutically acceptable salt thereof.

In another embodiment, the invention relates to a compound selected from the group consisting of

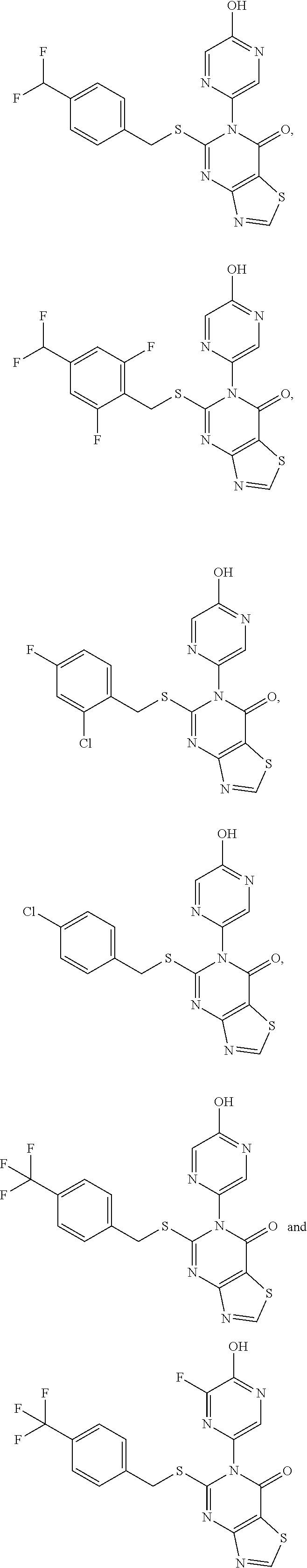

in its salt-free form or a salt thereof or a pharmaceutically acceptable salt thereof.

### Biological Assays

### Assay to Determine Inhibition of NOX4

### Cell Line Generation for NOX4 Inhibition Assay

The synthetic gene phNOX4_DNA3_1_Zeo (Accession: AAF68973) is assembled from synthetic oligonucleotides and/or PCR products. The fragment is cloned into pcDNA3.1_Zeo_A011 using NheI and XhoI cloning sites. The plasmid DNA is purified from transformed bacteria and its concentration is determined by UV spectroscopy. The final construct is verified by sequencing. The plasmid is transfected via electroporation (Amaxa electroporation device) in comibination with Nucleofector Kit V. Selection of NOX4 over-expressing genes is achieved using selection antibiotic Zeocin.

### NOX4 Inhibition Assay (HyPerBlu™)

The inhibitory activity of the example compounds of the invention is determined using the following procedure:

NOX4 inhibition is assessed utilizing HEK293 cells stably overexpressing human NOX4 (hNOX4), generating constitutively high levels of hydrogen peroxide (H₂O₂). Cells are cultured in the Dulbecco's Modified Eagle Medium (DMEM) media containing 4.5 g/L glucose supplemented with 10% fetal calf serum and 250 μg/ml Zeocin in an incubator at 37° C. with 5% CO₂. For the assay, cells are seeded in 384 well plates. After 24 h cells are washed and treated with several concentrations (10 nM-30 μM) of the test compounds (diluted in DMSO) or 100 μM diphenyleneiodonium chloride (DPI, used as positive control) in assay buffer consisting of phosphate-buffered saline (PBS) with 4-(2-hydroxyethyl)-1-pipera-zineethanesulfonic acid (HEPES), and incubated for 2 hours at 24° C. in a humidified incubator. After incubation Lumigen HyPerBlu™ is added and cells are incubated for additional 30 min at 24° C. in a humidified incubator. Next, luminescence is measured using an Envision Multimode Plate reader to determine levels of produced H₂O₂ in the wells. Results are visible in Table 1.

TABLE 1

| Example | IC₅₀ (NOX4) |
|---|---|
| Number | [nM] |
| 1 | 53 |
| 2 | 114 |
| 3 | 838 |
| 4 | 243 |
| 5 | 51 |
| 6 | 36 |
| 7 | 77 |
| 8 | 56 |
| 9 | 34 |
| 10 | 52 |
| 11 | 62 |
| 12 | 25 |
| 13 | 50 |
| 14 | 57 |
| 15 | 52 |
| 16 | 25 |
| 17 | 31 |
| 18 | 22 |
| 19 | 40 |
| 20 | 34 |
| 21 | 232 |
| 22 | 67 |
| 23 | 66 |
| 24 | 90 |
| 25 | 60 |
| 26 | 51 |
| 27 | 23 |
| 28 | 181 |
| 29 | 77 |
| 30 | 77 |
| 31 | 133 |
| 32 | 41 |
| 33 | 38 |
| 34 | 62 |
| 35 | 49 |
| 36 | 71 |
| 37 | 44 |
| 38 | 29 |
| 39 | 174 |
| 40 | 89 |
| 41 | 63 |
| 42 | 50 |
| 43 | 41 |
| 44 | 64 |
| 45 | 73 |
| 46 | 58 |
| 47 | 172 |
| 48 | 24 |
| 49 | 47 |
| 50 | 37 |
| 51 | 175 |
| 52 | 112 |
| 53 | 54 |
| 54 | 196 |
| 55 | 60 |
| 56 | 33 |
| 57 | 101 |
| 58 | 28 |
| 59 | 45 |
| 60 | 55 |
| 61 | 64 |
| 62 | 81 |
| 63 | 136 |
| 64 | 176 |
| 65 | 288 |
| 66 | 111 |
| 67 | 85 |
| 68 | 25 |
| 69 | 53 |
| 70 | 46 |
| 71 | 58 |
| 72 | 47 |
| 73 | 50 |
| 74 | 69 |
| 75 | 84 |
| 76 | 86 |
| 77 | 88 |
| 78 | 35 |
| 79 | 54 |
| 80 | 69 |
| 81 | 75 |
| 82 | 83 |
| 83 | 57 |
| 84 | 84 |
| 85 | 113 |
| 86 | 143 |
| 87 | 180 |

### Assays to Determine the Selectivity Against NOX1, NOX2, NOX3, NOX5

### Cell Line Generation

Plasmids are purchased from Vectorbuilder.
for HEK-hNOX1 all necessary subunits are cloned accordingly:
   pLV-CMV-hNOX1-puro (NM_007052.5)
   pLV-CMV-hNOXO1-blas (NM_172168.3)
   pLV-CMV-hp22phox-hygro (NM_000101.4)
   pLV-CMV-hNOXAl-neo (NM_006647.2)
for HEK-hNOX3
   pLV-CMV-hNOX3-puro (NM_015718.3)

3rd generation lentiviral particles are produced in HEK293 suspension cells by transient transfection of three helper and expression plasmids. Crucial lentivirus supernatants are harvested, clarified by filtration and concentrated by precipitation. Lentivirus titers are determined in transducing units in (TU/mL) by transduction of HT1080 cells and colony forming assay. Parental HEK293 cells, purchased from CLS GmbH, are transduced with replica-tion-incompetent 3rd generation lentiviral particles (MOI 2) and expanded for at least two weeks before cryopreservation of tested lots. During cultivation, medium is changed three times per week and cells are sub-cultured at least once per week. Selection antibiotics are added according to the plasmids (1 μg/ml puromycin, 1.5 μg/ml blasticidin, 200 μg/ml G418 and 100 μg/ml hygromycin).

### For HEK-hNOX5

The synthetic gene hNOX5 (Accession: Q96PH1) is cloned into pcDNA3.1_Zeo. The plasmid DNA is purified from transformed bacteria and its concentration is determined by UV spectroscopy. The final construct is verified by sequencing. The plasmid is transfected via electroporation (Amaxa electroporation device) in comibination with Nucleofector Kit V. Selection of NOX5 overexpressing genes is achieved using selection antibiotic Zeocin.

### NOX1 Inhibition Assay (L-012)

Evaluation of the NOX4 inhibitors on NOX1 activity is assessed in HEK293 cells stably overexpressing human NOX1 (hNOX1).

Cells are cultured in the DMEM media containing 4.5 g/L glucose supplemented with 10% fetal calf serum (FCS), Puromycin1 μg/mL, Blasticidin 1.5 μg/mL, Geneticin (G418) 200 μg/mL and Hygromycin 100 μg/mL in an incubator at 37° C. with 5% CO₂. For the assay cells are seeded in 384 well plates in DMEM media with 10% FCS. After 24 h, cells are washed, treated for 30 mins with several concentrations (30 nM-100 μM) of test compounds and then stimulated with phorbol myristate acetate (PMA) at 1 μM to induce NOX1-dependent ROS production (except wells used as a positive control). Lastly, 8-amino-5-chloro-2,3-dihydro-7-phenyl-pyrido[3,4-d]pyridazine-1,4-dione sodium salt (L-012, CAS 143556-24-5) is added at 400 μM to all wells, and cells are incubated in an incubator for an additional 3 h. Afterwards luminescence is measured with a SpectraMax Paradigm Microplate Reader. Data can be found in table 2.

### NOX2 Inhibition Assay (L-012)

Evaluation of the NOX4 inhibitors on NOX2 activity is assessed in human blood-derived granulocytes.

Different concentrations of the test compounds (30 nM-100 μM), DMSO (as a negative control) or diphenyleneidonium chloride (DPI; 30 μM, as a positive control), prepared in assay buffer (PBS containing CaCl₂) and MgCl2+0.1% bovine serum albumin (BSA)), are placed in 384 well plates. Afterwards, freshly isolated granulocytes are added to the wells containing the compounds in assay buffer and incubated for 1 h at 37° C. Next, a solution of N-formylmethionine-leucyl-phenylalanine (fMLP; 730 nM), phorbol myristate acetate (PMA, 100 nM) (stimuli of ROS production) and L-012 (200 uM, ROS indicator) is added to the wells and the cells are incubated for additional 30 mins at 37° C. Afterwards luminescence is measured in a SpectraMax M5 microplate reader. Data can be found in table 2.

### NOX3 Inhibition Assay (L-012)

Evaluation of the NOX4 inhibitors on NOX3 activity is assessed in HEK293 cells stably overexpressing human NOX3 (hNOX3).

Cells are cultured in the DMEM media containing 4.5 g/L glucose supplemented with 10% fetal calf serum (FCS), Puromycin 1 μg/mL and Blasticidin 1.5 μg/mL in an incubator at 37° C. with 5% CO₂. Cells are seeded in 384 well plates for 2 days. For the assay cells are washed and treated with several concentrations (10 nM-30 μM) of test compound (diluted in DMSO) or 100 μM DPI (as a positive control in assay buffer consisting of PBS), and incubated for 2 hours at 24° C. in a humidified incubator. Cells are then stimulated with PMA at 0.1 μM to induce NOX3-dependent ROS production. Finally, L-012 is added at 100 μM and incubated for 2 hours at 24° C. in a humidified incubator. Luminescence is measured with an PHERAstar multimode reader. Data can be found in table 2.

### NOX5 Inhibition Assay (L-012)

Evaluation of the NOX4 inhibitors on NOX5 activity is assessed in HEK293 cells stably overexpressing human NOX5 (hNOX5).

Cells are cultured in the DMEM media containing 4.5 g/L glucose supplemented with 10% fetal calf serum and 250 μg/ml Zeocin in an incubator at 37° C. with 5% CO₂. Cells are seeded in 384 well plates over-night. For the assay cells are washed and treated with several concentrations (10 nM-30 μM) of test compounds (diluted in DMSO and added to assay buffer consisting of PBS) or 100 μM DPI as a positive control and incubated for 2 hours at 24° C. in a humidified incubator. Cells are then stimulated with PMA at 0.1 μM to induce NOX5-dependent ROS production. Finally, L-012 is added at 100 μM and incubated for 2 hours at 24° C. in a humidified incubator. Luminescence is measured with an PHERAstar multimode reader. Data can be found in table 2.

TABLE 2

| Example | NOX1 IC₅₀ | NOX2 IC₅₀ | NOX3 IC₅₀ | NOX5 IC₅₀ |
|---|---|---|---|---|
| Number | [μM] | [μM] | [μM] | [μM] |
| 1 | 5 | >100 | 27 | 35 |
| 2 | N/A | >100 | N/A | N/A |
| 4 | 32 | >100 | >100 | 28 |
| 6 | >100 | >100 | >100 | >100 |
| 7 | >100 | >100 | >100 | >100 |
| 8 | >100 | >100 | >100 | >100 |
| 9 | >100 | 88 | 10 | >100 |
| 10 | 57 | >100 | 28 | >100 |
| 11 | 66 | >100 | >100 | >100 |
| 12 | 44 | >100 | 25 | 61 |
| 13 | 52 | >100 | 18 | 120 |
| 14 | >100 | >100 | >100 | >100 |
| 15 | >100 | >100 | 31 | >100 |
| 16 | >100 | >100 | 19 | >100 |
| 17 | 56 | >100 | 47 | 86 |
| 18 | 32 | >100 | 2.8 | 65 |
| 19 | 9.3 | >100 | 3 | 44 |
| 20 | 31 | >100 | N/A | N/A |
| 21 | 36 | >100 | N/A | N/A |
| 22 | 60 | >100 | >100 | >100 |
| 23 | >100 | 84 | >100 | >100 |
| 24 | 19 | >100 | 27 | >100 |
| 25 | 30 | >100 | >100 | >100 |
| 26 | 38 | 188 | 91 | 81 |
| 27 | 68 | >100 | 2 | 86 |
| 28 | 72 | >100 | >100 | >100 |
| 29 | 17 | >100 | 7 | 25 |
| 30 | 29 | >100 | 4.2 | >100 |
| 31 | 30 | >100 | 12 | >100 |
| 32 | 55 | >100 | 12 | >100 |
| 33 | 27 | >100 | 4 | 30 |
| 34 | 65 | >100 | 38 | 87 |
| 35 | 40 | >100 | 12 | 74 |
| 36 | 34 | >100 | 2 | 32 |
| 37 | 44 | >100 | 21 | >100 |
| 38 | 38 | >100 | 1 | >100 |
| 39 | 42 | >100 | 84 | >100 |
| 40 | 32 | >100 | >100 | >100 |
| 41 | 49 | >100 | 5 | >100 |
| 42 | 30 | >100 | 1 | >100 |
| 43 | 18 | >100 | 7 | 69 |
| 44 | 5 | >100 | 2 | 22 |
| 45 | 37 | >100 | 23 | 62 |
| 46 | 86 | >100 | >100 | >100 |
| 47 | >100 | >100 | 64 | >100 |
| 48 | >100 | >100 | 6 | 76 |
| 49 | 26 | >100 | 2 | 43 |
| 50 | >100 | >100 | 3 | >100 |
| 51 | >100 | >100 | >100 | >100 |
| 52 | 95 | N/A | 1 | 85 |
| 53 | 72 | >100.0 | 13 | >100 |
| 54 | 28 | >100 | >100 | >100 |
| 55 | 19 | >100 | 35 | 43 |
| 56 | 30 | >100 | 100 | 35 |
| 57 | 58 | >100 | >100 | >100 |
| 58 | >100 | >100 | 37 | >100 |
| 59 | >100 | >100 | 33 | 53 |
| 60 | >100 | >100 | 34 | 48 |
| 61 | 66 | >100 | >100 | >100 |
| 62 | >100 | >100 | >100 | 68 |
| 63 | N/A | N/A | N/A | N/A |
| 64 | 66 | >100 | 36 | 61 |
| 65 | N/A | N/A | N/A | N/A |
| 66 | 10 | >100 | 51 | 37 |
| 67 | 74 | >100 | 52 | 63 |
| 68 | 46 | >100.0 | >100 | 52 |
| 69 | 9.8 | >100 | >100 | >100 |
| 70 | 59 | >100 | 63 | 94 |
| 71 | 30 | >100 | 32 | 21 |
| 72 | 68 | >100 | >100 | 100 |
| 73 | 33 | >100 | >100 | >100 |
| 74 | >100 | >100 | >100 | >100 |
| 75 | >100 | >100 | >100 | >100 |
| 76 | 22 | >100 | 49 | 23 |
| 77 | 87 | >100 | >100 | >100 |
| 78 | >100 | >100 | 55 | 89 |
| 79 | >100 | >100 | 19 | >100 |
| 80 | >100 | >100 | 57 | 52 |
| 81 | >100 | >100 | >100 | >100 |
| 82 | >100 | >100 | 39 | >100 |
| 83 | 63 | >100.0 | >100 | 84 |
| 84 | N/A | N/A | >100 | 130 |
| 85 | >100 | >100 | >100 | 88 |
| 86 | >100 | N/A | >100 | 110 |
| 87 | 79 | N/A | >100 | 50 |

*N/A: not available

### Evaluation of Hepatocyte Clearance

The metabolic degradation of the test compound is assayed in a hepatocyte suspension. Hepatocytes (cryopreserved) are incubated in Dulbecco's modified eagle medium (supplemented with 3.5 μg glucagon/500 mL, 2.5 mg insulin/500 mL and 3.75 mg/500 mL hydrocorti-sone) containing 5% human serum.

Following a 30 min preincubation in an incubator (37° C., 10% CO₂) 5 μl of test compound solution (80 μM; from 2 mM in DMSO stock solution diluted 1:25 with medium) are added into 395 μl hepatocyte suspension (cell density in the range 0.25-5 Mio cells/mL, typically 1 Mio cells/mL; final concentration of test compound 1 μM, final DMSO concentration 0.05%).

The cells are incubated for six hours (incubator, orbital shaker) and samples (25 μl) are taken at 0, 0.5, 1, 2, 4 and 6 hours. Samples are transferred into acetonitrile and pelleted by cen-trifugation (5 min). The supernatant is transferred to a new 96-deepwell plate, evaporated under nitrogen and resuspended.

Decline of parent compound is analyzed by HPLC-MS/MS.

CLᵢₙₜ is calculated as follows:

$$CL\_INTRINSIC - Dose / AUC = ( C 0 / CD ) / ( AUD + clast / k ) \times 1000 / 60.$$

C0: initial concentration in the incubation [μM], CD: cell density of vital cells [10⁶ cells/mL], AUD: area under the data [μM×h], clast: concentration of last data point [μM],
k: slope of the regression line for parent decline [h−1].

The calculated in vitro hepatic intrinsic clearance can be scaled up to the intrinsic in vivo hepatic clearance and used to predict hepatic in vivo blood clearance (CL) by the use of a liver model (well stirred model).

$$CL\_INTRINSIC \_INVIVO = [ ml / min / kg ] = ( CL\_INTRINSIC [ μL / min / {10}^{6} cells ] \times hepatocel - lularity [ {10}^{6} cells / g liver ] \times liver factor [ g / kg body weight ] ) / 1000$$

$$CL [ ml / min / kg ] = CL\_INTRINSIC \_INVIVO [ ml / min / kg ] \times hepatic blood flow [ ml / min / kg ] / ( CL\_INTRINSIC \_INVIVO [ ml / min / kg ] + hepatic blood flow [ ml / min / kg ] )$$

$$Qh [ \% ] = CL [ ml / min / kg ] / hepatic blood flow [ ml / min / kg ] )$$

Hepatocellularity, human: 120×10⁶ cells/g liver
Liver factor, human: 25.7 g/kg bodyweight
Blood flow, human: 21 ml/(min×kg)

The resulting data are visible in table 3.

TABLE 3

| Example | |
|---|---|
| Number | Qh [%] |
| 1 | 59 |
| 2 | 38 |
| 6 | 12 |
| 7 | 12 |
| 8 | 17 |
| 9 | 12 |
| 10 | 26 |
| 11 | 8 |
| 12 | 14 |
| 13 | 9 |
| 14 | 9 |
| 15 | 15 |
| 16 | 18 |
| 17 | 10 |
| 18 | 24 |
| 19 | 11 |
| 20 | 12 |
| 22 | 30 |
| 23 | 19 |
| 24 | 15 |
| 25 | 12 |
| 26 | 19 |
| 27 | 8 |
| 30 | 22 |
| 34 | 10 |
| 35 | 32 |
| 36 | 15 |
| 37 | 32 |
| 38 | 7 |
| 42 | 13 |
| 43 | 8 |
| 46 | 26 |
| 48 | 13 |
| 49 | 22 |
| 50 | 34 |
| 53 | 5 |
| 55 | 13 |
| 56 | 10 |
| 57 | 14 |
| 58 | 8 |
| 59 | 15 |
| 60 | 11 |
| 61 | 13 |
| 62 | 10 |
| 63 | 12 |
| 66 | 15 |
| 67 | 21 |
| 68 | 35 |
| 69 | 9 |
| 70 | 21 |
| 71 | 14 |
| 72 | 11 |
| 73 | 6 |
| 74 | <4 |
| 75 | 13 |
| 76 | 5 |
| 77 | 10 |
| 78 | 18 |
| 79 | 23 |
| 80 | 16 |
| 81 | 33 |
| 82 | 19 |
| 83 | 15 |
| 84 | 13 |
| 85 | 9 |

### Method of Treatment

The present invention is directed to compounds of general formula (I) which are useful in the prevention and/or treatment of a disease and/or condition associated with or modulated by NOX4 activity, including but not limited to the treatment and/or prevention of chronic liver diseases, portal hypertension, viral infections, cancer, interstitial lung diseases, retinopathies, acute and chronic inflammation as well as fibrotic diseases. Particularly, the pharmaceutical compositions of the invention are suitable for the therapy of interstitial lung diseases, e.g. idiopathic pulmonary disease, and may also be suitable for the therapy of fibrotic diseases, allergic and inflammatory diseases.

The compounds of general formula (I) are useful for the prevention and/or treatment of: vascular inflammation, atherosclerosis, interstitial lung diseases (e.g. idiopathic pulmonary fibrosis, progressive pulmonary fibrosis), liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, head and neck tumors), systemic sclerosis, inflammatory bowel disease, Duchenne muscular dystrophy, COVID-19, acute respiratory distress syndrome, influenza, ischemic and hemorrhagic stroke.

Accordingly, the present invention relates to a compound of general formula (I) for use as a medicament.

Furthermore, the present invention relates to the use of a compound of general formula (I) for the treatment and/or prevention of a disease and/or condition associated with or modulated by NOX4 activity.

Furthermore, the present invention relates to the use of a compound of general formula (I) for the treatment and/or prevention of chronic liver diseases, viral infections, cancer, interstitial lung diseases, retinopathies, acute and chronic inflammation as well as fibrotic diseases.

Particularly, the pharmaceutical compositions of the invention are suitable for the therapy of interstitial lung diseases, e.g. idiopathic pulmonary disease, and may also be suitable for the therapy of fibrotic, allergic and inflammatory diseases.

Furthermore, the present invention relates to the use of a compound of general formula (I) for the treatment and/or prevention of: vascular inflammation, atherosclerosis, interstitial lung diseases, e.g. idiopathic pulmonary fibrosis, progressive pulmonary fibrosis, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, and head and neck tumors), systemic sclerosis, inflammatory bowel disease, Duchenne muscular dystrophy, COVID-19, acute respiratory distress syndrome, influenza, pulmonary hypertension, ischemic and hemorrhagic stroke, heart failure, cardio myopathies, hypertension, progression of interstitial lung diseases, chronic lung disease, chronic obstructive pulmonary disease (COPD), asthma, diabetic nephropathy, acute kidney injury, obstructive nephropathy, hypertensive nephropathy, and neurodegenerative diseases.

In a further aspect the present invention relates to a compound of general formula (I) for use in the treatment and/or prevention of above mentioned diseases and conditions.

In a further aspect the present invention relates to the use of a compound of general formula (I) for the preparation of a medicament for the treatment and/or prevention of above mentioned diseases and conditions.

In a further aspect of the present invention the present invention relates to methods for the treatment or prevention of above mentioned diseases and conditions, which method comprises the administration of an effective amount of a compound of general formula (I) to a human being.

The dose range of the compounds of general formula (I) applicable per day is usually from 0.00001 to 100 mg per kg body weight, for example from 0.00001 to 10 mg per kg body weight of the patient. Each dosage unit may conveniently contain from 0.001 to 1000 mg, for example from 0.001 to 100 mg.

The actual pharmaceutically effective amount or therapeutic dosage will usually depend on factors known by those skilled in the art such as age and weight of the patient, route of administration and severity of disease. In any case the compounds will be administered at dosages and in a manner which allows a pharmaceutically effective amount to be delivered based upon patient's unique condition.

### Pharmaceutical Composition

Suitable preparations for administering the compounds of formula (I) will be apparent to those with ordinary skill in the art and include for example tablets, pills, capsules, suppositories, lozenges, troches, solutions, syrups, elixirs, sachets, injectables, inhalables and powders etc.

Suitable tablets may be obtained, for example, by mixing one or more compounds according to formula (I) with known excipients, for example inert diluents, carriers, disintegrants, adjuvants, surfactants, binders and/or lubricants.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with chronic liver diseases, viral infections, cancer, interstitial lung diseases, retinopathies, acute and chronic inflammation as well as fibrotic diseases.

According to another embodiment, the additional therapeutic agent is selected from the group of therapeutic agents useful in the treatment of diseases or conditions described hereinbefore, in particular associated with vascular inflammation, atherosclerosis, interstitial lung diseases (e.g. idiopathic pulmonary fibrosis, progressive pulmonary fibrosis), liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, head and neck tumors), systemic sclerosis, inflammatory bowel disease, Duchenne muscular dystrophy, COVID-19, acute respiratory distress syndrome, influenza, ischemic and hemorrhagic stroke.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of antifibrotics (e.g. Ofev, PDE4i); of immunotherapeutics (e.g. PD-1, aCTLA-4); of T cell engagers; of sGC activators; of ATX-inhibitors; of SGLT2 inhibitors (e.g. dapagliflozin, empagliflozin); of THRb inhibitors; of GLP1 agonists and GLP1 agonist combinations; of FGF-analogs, such as FGF21 or FGF19; of KRAS-G12C-inhibitors (e.g. sotorasib); of KRAS-G12D-inhibitors; of MDM2-p53-antagonists; of Her2-inhibitors; of platinum derivatives (e.g. cisplatin, oxaliplatin, carboplatin); of alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); of antimitotic agents (e.g. Vinca alkaloids e.g. vinblastine, vindesin, vinorelbin and vincristine); of taxanes such as paclitaxel, docetaxel, nab-paclitaxel (Abraxane); of angiogenesis inhibitors (e.g. tasquinimod, bevacizumab); of tubuline inhibitors; of DNA synthesis inhibitors; of PARP inhibitors; of topoisomerase inhibitors (e.g. epipodophyllotoxins such as for example etoposide and etopophos, teniposide, amsacrin, topotecan, irinotecan, mitoxantrone); of antimetabolites (e.g. methotrexate, raltitrexed, 5-fluorouracil (5-FU), capecitabine, floxuridine, gemcitabine, mercaptopurine, thioguanine, cladribine, pentostatin, cytarabine (ara C), fludarabine, combination of trifluridine and tipiracil (=TAS102)); of antitumor antibiotics (e.g. anthracyclins such as doxorubicin, doxil (pegylated liposomal doxorubicin hydrochloride), myocet (non-pegylated liposomal doxorubicin), daunorubicin, epirubicin and idarubicin, mitomycin-C, bleomycin, dactinomycin, plicamycin, streptozocin); of inhibitors of vascular endothelial growth factor. Furthermore, the compounds according to the present invention can be combined with a radiotherapy regime.

Therefore, in another aspect, this invention relates to the use of a compound according to the invention in combination with one or more additional therapeutic agents described hereinbefore and hereinafter for the treatment of diseases or conditions which may be affected or which are mediated by NOX4, in particular diseases or conditions as described hereinbefore and hereinafter.

In a further aspect this invention relates to a method for treating a disease or condition which can be influenced by the inhibition of NOX4 in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of NOX4 in a patient in need thereof.

In yet another aspect the present invention relates to a method for the treatment of a disease or condition mediated by NOX4 activity in a patient that includes the step of administering to the human patient, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### Preparation

The compounds according to the invention and their intermediates may be obtained using methods of synthesis which are known to the one skilled in the art and described in the literature of organic synthesis. Preferably the compounds are obtained analogously to the methods of preparation explained more fully hereinafter, in particular as described in the experimental section. In some cases, the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the literature. The terms “ambient temperature” and “room temperature” are used interchangeably and designate a temperature of about 20° C., e.g. between 19 and 24° C.

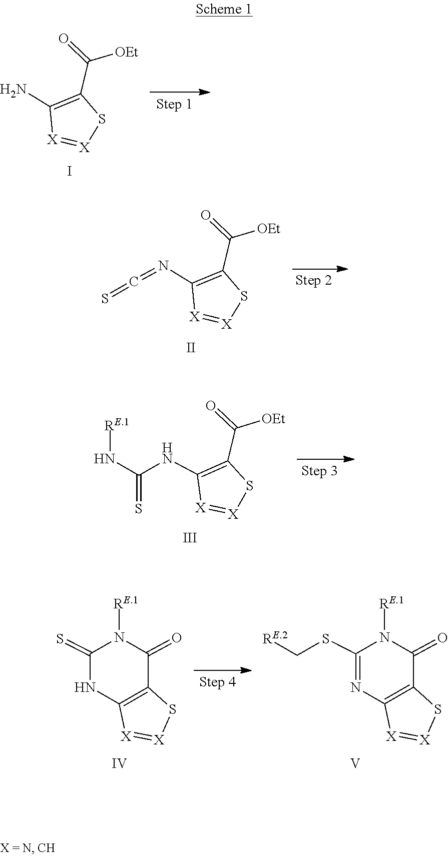

Intermediates II (Step 1, intermediates I→intermediates II) can be prepared by treating intermediates I with a suitable thiocarbonylation reagent, for example 1,1′-thiocarbonylbis(pyridin-2(1H)-one), in a suitable solvent, for example acetonitrile (Scheme 1). Preferred reaction temperatures are between room temperature and 75° C. Formation of the thiourea (Step 2, intermediates II→intermediates III) can be achieved by reacting intermediates II with an aniline-R^{E.1}—NH₂ either in the presence of a suitable base, such as triethylamine or N,N-diisopropylethylamine, or without base, in a suitable solvent, such as acetonitrile or 2-methyltetrahydrofuran. The cyclization (Step 3, intermediates III→intermediates IV) can be achieved by treating intermediates III with a suitable base, such as potassium tert-butoxide or aqueous sodium hydroxide in an appropriate solvent, such as acetonitrile, methanol, or water. Compounds according to the present invention V (step 4, intermediates IV→compounds of the invention V) can be prepared by reaction of intermediates IV with a benzyl halide (i.e. chloride or bromide) R^{E.2}—CH₂—Z (Z is halide) and a suitable base, such as N,N-diisopropylethylamine or triethylamine, in a suitable solvent, for example acetonitrile, propan-2-ol, N,N-dimethylacetamide or N,N-dimethylformamide.

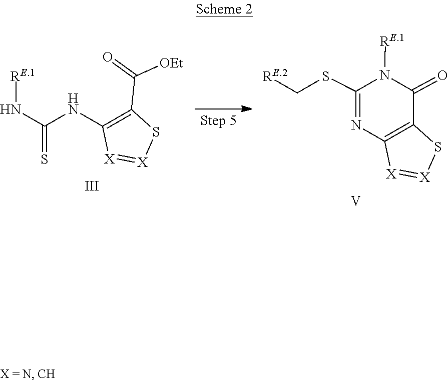

Alternatively, compounds of the present invention V can directly be obtained by reacting intermediates III with a benzyl halide, such as benzyl chloride or benzyl bromide, and a suitable base, such as N,N-diisopropylethylamine or aqueous lithium hydroxide in a suitable solvent, such as N,N-dimethylformamide or tetrahydrofuran, at temperatures between room temperature and 40° C. (Scheme 2).

Abbreviations:

| | |
|---|---|
| ACN | acetonitrile |
| Aq. | aqueous |
| Boc | tert-butyloxycarbonyl |
| ° C. | degree celsius |
| CDI | carbonyl diimidazole |
| CyH/CH | cyclohexane |
| conc. | concentrated |
| DCM | dichloromethane |
| DIPEA | N,N-diisopropylethylamine |
| DMA | N,N-dimethylacetamide |
| DMF | N,N-dimethylformamide |
| DMSO | dimethyl sulfoxide |
| ESI-MS | electrospray ionisation mass spectrometry |
| EtOAc | ethyl acetate |
| EtOH | ethanol |
| ex | example |
| equiv | equivalent |
| FA | formic acid |
| h | hour |
| HATU | 1-[bis(dimethylamino)methylene]-1H-1,2,3- |
| | triazolo[4,5-b]pyridinium 3-oxid hexafluorophosphate |
| HCl | hydrochloric acid |
| HPLC | high performance liquid chromatography |
| Int. | intermediate |
| K₂CO₃ | potassium carbonate |
| KotBu | potassium tert-butoxide |
| L | liter |
| LiOH*H₂O | lithium hydroxide monohydrate |
| M | Molar (mol/L) |
| MeOH | methanol |
| MgSO₄ | magnesium sulphate |
| min | minute |
| mL | milliliter |
| MTBE | tert-butylmethylether |
| MW | molecular weight |
| NaOEt | sodium ethoxide |
| NH₃ | ammonia |
| PMB | para-methoxybenzyl |
| Prep. | Preparative |
| RP | reversed phase |
| R_{f} | Retardation factor |
| Rₜ | retention time |
| sat. | saturated |
| scCO₂ | supercritical CO₂ |
| SFC | supercritical fluid chromatography |
| TBTU | 2-(1H-benzotriazole-1-yl)-1,1,3,3-tetramethylaminium |
| | tetrafluoroborate |
| TCDI | 1,1-thiocarbonyldiimidazole |
| TEA | triethylamine |
| TFA | trifluoroacetic acid |
| TFAA | trifluoroacetic anhydride |
| THF | tetrahydrofuran |
| TBS | tert-butyldimethylsilyl |
| TMS | trimethylsilyl |
| TMS-Cl | trimethylsilyl chloride |
| XphosPdG3 | (2-dicyclohexylphosphino-2′,4′,6′-triisopropyl-1,1′- |
| | biphenyl)[2-(2′-amino-1,1′-biphenyl)]palladium(II) |
| | methanesulfonate |
| XPhosPdG4 | methanesulfonato(2-dicyclohexylphosphino-2′,4′,6′-tri-i- |
| | propyl-1,1′-biphenyl)(2′-methylamino-1,1′-biphenyl-2- |
| | yl)palladium(II) |

### Nomenclature

The names of the compounds described in the experimental section were generated using PerkinElmer ChemDraw© Software (Version 22.2.0.330).

### Preparation of Starting Materials

Unless stated otherwise, the starting materials are commercially available and used without further purification.

### PREPARATION OF INTERMEDIATES

### Intermediate 1

### Ethyl 4-isothiocyanatothiazole-5-carboxylate

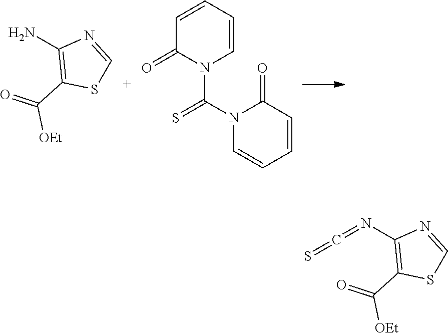

1,1′-Thiocarbonylbis(pyridin-2(1H)-one) (28.3 g, 122 mmol, 1.10 equiv) is added to a solution of ethyl 4-aminothiazole-5-carboxylate (20.0 g, 116 mmol, 1 equiv) in acetonitrile (110 mL), and the reaction mixture is heated at 75° C. After 3 h, the reaction mixture is poured on ice-water, and the mixture is extracted with tert-butylmethylether. The phases are separated, and the organic phase is washed with water and dried over sodium sulfate. All volatiles are removed under reduced pressure, and the residue is purified by flash column chromatography (silica gel, gradient cyclohexane to cyclohexane/EtOAc 70:30) to provide the product.

| | |
|---|---|
| C₇H₆N₂O₂S₂ | (MW = 214.0 g/mol) |
| ESI | 215 [M + H]⁺ |
| Rₜ (HPLC) | 0.96 min (Z018_S04) |

### Intermediate 2

### 6-Fluoro-5-methoxypyrazin-2-amine

### Step 1

### 5-Bromo-3-fluoro-2-methoxypyrazine

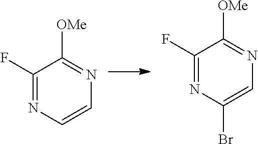 [SMILES: COc1nccnc1F ; COc1ncc(Br)nc1F]

3-Nitrobezenesulfonic acid (150 mg, 724 μmol, 0.100 equiv) and 1,3-dibromo-5,5-dimethylhydantoin (1.30 g, 4.41 mmol, 0.600 equiv) are added to a solution of 2-fluoro-3-methoxypyrazine (1.00 g, 7.42 mmol, 1 equiv) in 1,1,1,3,3,3-hexafluoropropan-2-ol (5 mL), and the reaction mixture is heated at 60° C. After 18 h, the reaction mixture is concentrated under reduced pressure (50° C., 84 mbar). The residue is dissolved in dichloromethane, and the organic phase is washed with water. The organic layer is concentrated under reduced pressure (50° C., 84 mbar), and the residue is purified by flash column chromatography (silica gel, gradient cyclohexane to cyclohexane/EtOAc 90:10) to yield the desired product.

| | |
|---|---|
| C₅H₄BrFN₂O | (MW = 205.9 g/mol) |
| ESI | not detected |
| Rₜ (HPLC) | 0.93 min (Z018_S04) |

### Step 2

### 6-Fluoro-5-methoxypyrazin-2-amine

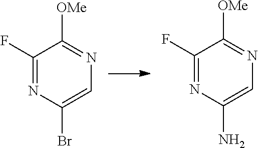 [SMILES: COc1ncc(Br)nc1F ; COc1ncc(N)nc1F]

Under an atmosphere of nitrogen, a solution of lithium bis(trimethylsilyl)amide (1 M in THF, 4.35 mL, 4.35 mmol, 3.00 equiv) is added dropwise to a mixture of 5-bromo-3-fluoro-2-methoxypyrazine (step 1, 300 mg, 1.45 mmol, 1 equiv) and bis(di-tert-butyl(4-dimethylaminophenyl)phosphine)dichlorpalladium(II) (67.0 mg, 145 μmol, 0.10 equiv) in 1,4-dioxane (6 mL). After complete addition, the reaction mixture is heated at 110° C. After 18 h, the reaction mixture is cooled at room temperature and concentrated under reduced pressure (50° C., 180 mbar). The residue is purified by flash column chromatography (silica gel, gradient cyclohexane to cyclohexane/EtOAc 65:35) to yield the desired product.

| | |
|---|---|
| C₅H₆FN₃O | (MW = 143.0 g/mol) |
| ESI | 144 [M + H]⁺ |
| Rₜ (HPLC) | 0.58 min (Z018_S04) |

### Intermediate 3

### 6-(6-Fluoro-5-methoxypyrazin-2-yl)-5-((4-(trifluoromethyl)benzyl)thio)thiazolo[4,5-d]pyrimidin-7(6H)-one

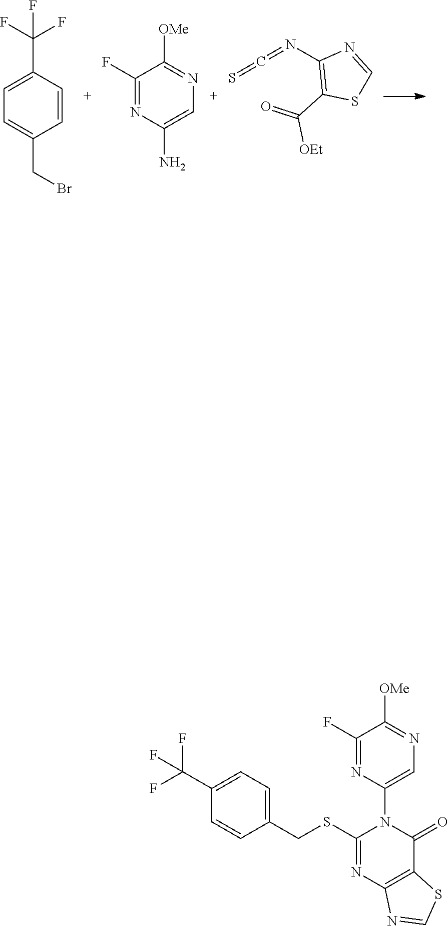

A mixture of ethyl 4-isothiocyanatothiazole-5-carboxylate (intermediate 1, 150 mg, 700 μmol, 1 equiv), and 6-fluoro-5-methoxypyrazin-2-amine (intermediate 2, 100 mg, 699 μmol, 1 equiv) in acetonitrile (3 mL) is stirred for 24 h at room temperature. Potassium tert-butoxide (78.0 mg, 695 μmol, 1 equiv) is added, and the mixture is stirred for 4 h. 1-(Bromomethyl)-4-(trifluoromethyl)benzene (170 mg, 697 μmol, 1 equiv) is added, and the mixture is stirred for 15 min. Dichloromethane and water are added, and the layers are separated. The organic layer is concentrated under reduced pressure. The residue is purified by reversed phase HPLC (Agilent Sunfire™-C18, gradient of acetonitrile in water, 0.1% TFA) to yield the desired product.

| | |
|---|---|
| C₁₈H₁₁F₄N₅O₂S₂ | (MW = 469.0 g/mol) |
| ESI | 470 [M + H]⁺ |
| Rₜ (HPLC) | 1.10 min (Z018_S04) |

The following compounds are prepared using procedures analogous to those described for intermediate 3, using appropriate starting materials. As is appreciated by those skilled in the art, the analogous examples may involve variations in general reaction conditions.

| | | | HPLC retention time |
|---|---|---|---|
| Intermediate | Structure | ESI-MS | [min] (method) |
| 4 | 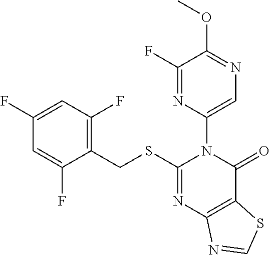 [SMILES: COc1ncc(-n2c(SCc3c(F)cc(F)cc3F)nc3ncsc3c2=O)nc1F] | 456 [M + H]⁺ | 1.07 (Z018_S04) |
| 5 | 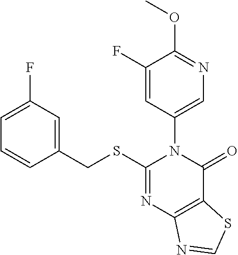 [SMILES: COc1ncc(-n2c(SCc3cccc(F)c3)nc3ncsc3c2=O)cc1F] | 401 [M + H]⁺ | 0.98 (Z018_S04) |
| 6 | 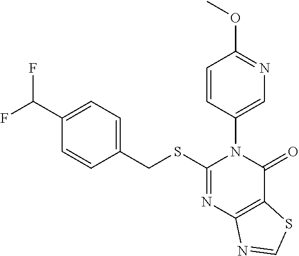 [SMILES: COc1ccc(-n2c(SCc3ccc(C(F)F)cc3)nc3ncsc3c2=O)cn1] | 433 [M + H]⁺ | 0.98 (Z018_S04) |

### Intermediate 7

### 5-Ethoxy-3-fluoropyrazin-2-amine

### Step 1

### Tert-butyl (5-ethoxypyrazin-2-yl)carbamate

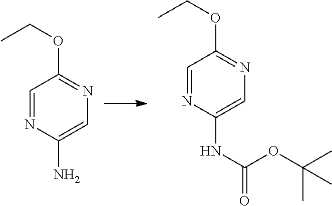

Under an atmosphere of nitrogen, 4-dimethylamino-pyridine (658 mg, 5.39 μmol, 0.100 equiv) is added to a mixture of 5-ethoxypyrazin-2-yl-amine (7.50 g, 53.9 mmol, 1 equiv) in tetrahydrofuran (5 mL), and the mixture is stirred at room temperature for 1 h. Di-tert-butyldicarbonate (17.6 g, 80.8 mmol, 1.50 equiv) is added, and the mixture is heated at reflux for 14 h. The mixture is cooled at room temperature and poured into water (5 mL). The mixture is extracted with ethyl acetate (3×), and the combined organic layers are concentrated under reduced pressure. The residue is purified by flash column chromatography (silica gel, gradient petroleum ether EtOAc 20:1 to petroleum ether/EtOAc 5:1) to yield the desired product.

| | |
|---|---|
| C₁₁H₁₇N₃O₃ | (MW = 239.3 g/mol) |
| R_{f} (silica gel) | 0.6 (dichloromethan/MeOH 20:1) |

### Step 2

### Tert-butyl (5-ethoxy-3-fluoropyrazin-2-yl)carbamate

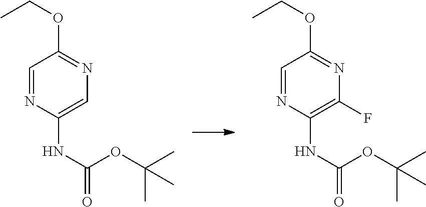 [SMILES: CCOc1cnc(NC(=O)OC(C)(C)C)cn1 ; CCOc1cnc(NC(=O)OC(C)(C)C)c(F)n1]

1-(Chloromethyl)-4-fluoro-1,4-diazabicyclo[2.2.2]octane-1,4-diium ditetrafluoroborate (Selectfluor) (5.70 g, 16.1 mmol, 3.90 equiv) is added to a solution of tert-butyl (5-ethoxypyrazin-2-yl)carbamate (step 1, 980 mg, 4.10 mmol, 1 equiv) in acetonitrile (10 mL). After 5 d, the reaction mixture is filtered, and the filtrate is concentrated under reduced pressure. The residue is purified by flash column chromatography (silica gel, gradient cyclohexane to cyclohexane/EtOAc 2:1) to yield the desired product.

| | |
|---|---|
| C₁₁H₁₆FN₃O₃ | (MW = 257.1 g/mol) |
| ESI | not detected |
| Rₜ (HPLC) | 0.97 min (Z018_S04) |

### Step 3

### 5-Ethoxy-3-fluoropyrazin-2-amine

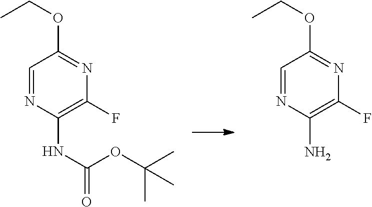 [SMILES: CCOc1cnc(NC(=O)OC(C)(C)C)c(F)n1 ; CCOc1cnc(N)c(F)n1]

Trifluoroacetic acid (1 mL) is added to a solution of tert-butyl (5-ethoxy-3-fluoropyrazin-2-yl)carbamate (step 3, 70.0 mg, 272 μmol, 1 equiv) in dichloromethane (2 mL). After 1 h, the reaction mixture is concentrated under reduced pressure to yield the desired product.

| | |
|---|---|
| C₆H₈FN₃O | (MW = 157.1 g/mol) |
| ESI | 158 [M + H]⁺ |
| Rₜ (HPLC) | 0.69 min (Z018_S04) |

### Intermediate 8

### 6-(5-Ethoxy-3-fluoropyrazin-2-yl)-5-thioxo-5,6-dihydrothiazolo[4,5-d]pyrimidin-7(4H)one

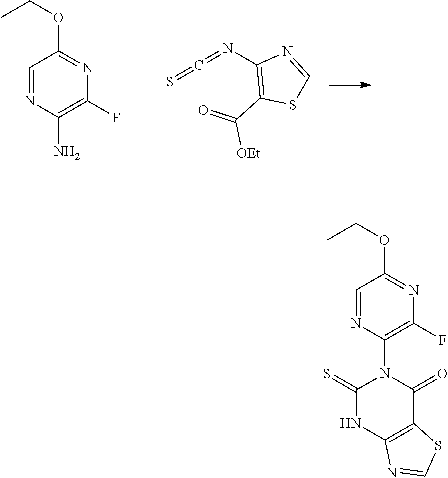 [SMILES: CCOc1cnc(N)c(F)n1 ; CCOC(=O)c1scnc1N=C=S ; CCOc1cnc(-n2c(=S)[nH]c3ncsc3c2=O)c(F)n1]

A mixture of ethyl 4-isothiocyanatothiazole-5-carboxylate (intermediate 1, 27.0 mg, 126 μmol, 1 equiv), and 5-ethoxy-3-fluoropyrazin-2-amine TFA salt (intermediate 7, 27.0 mg, 80.0 μmol, 0.600 equiv) in acetonitrile (2 mL) is stirred at room temperature. After 24 h, triethylamine (40 μL, 287 μmol, 2.30 equiv) is added. After 24 h, potassium tertbutoxide (55.0 mg, 490 μmol, 3.90 equiv) is added. After 30 min, the reaction mixture is acidified with acetic acid and purified by reversed phase HPLC (Agilent Sunfire™-C18, gradient of acetonitrile in water, 0.1% TFA) to yield the desired product.

| | |
|---|---|
| C₁₁H₈FN₅O₂S₂ | (MW = 325.0 g/mol) |
| ESI | 326 [M + H]⁺ |
| Rₜ (HPLC) | 0.86 min (Z018_S04) |

### Intermediate 9

### 2-Bromo-5-((4-methoxybenzyl)oxy)pyrazine

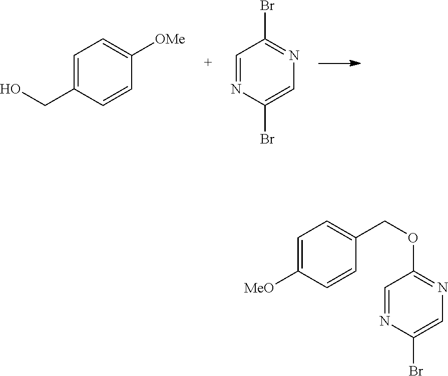 [SMILES: COc1ccc(CO)cc1 ; Brc1cnc(Br)cn1 ; COc1ccc(COc2cnc(Br)cn2)cc1]

Sodium hydride (60% in mineral oil, 9.90 g, 248 mmol, 1.10 equiv) is slowly added to a solution of 4-methoxybenzylalcohol (31.5 mL, 248 mmol, 1.10 equiv) in tetrahydrofuran (550 mL) keeping the temperature between 30-38° C. After 30 min, 2,5-dibromopyrazine (54.6 g, 225 mmol, 1 equiv) is added. After 1.5 h water is added, and the precipitate is filtered and dried to yield the desired product.

| | |
|---|---|
| C₁₂H₁₁BrN₂O₂S₂ | (MW = 294.0 g/mol) |
| ESI | 295/297 [M + H]⁺ (bromine isotope pattern) |
| Rₜ (HPLC) | 0.67 min (X012_S01) |

### Intermediate 10

### 5-((4-Methoxybenzyl)oxy)pyrazin-2-amine

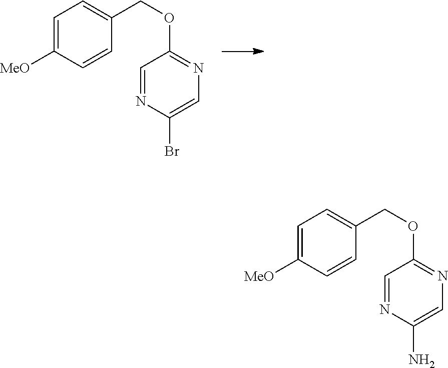 [SMILES: COc1ccc(COc2cnc(Br)cn2)cc1 ; COc1ccc(COc2cnc(N)cn2)cc1]

A solution of lithium-bis-(trimethylsilyl)-amide in tetrahydrofuran (1 M, 163 mL, 163 mmol, 1.20 equiv) is added dropwise to a mixture of 2-bromo-5-((4-methoxybenzyl)oxy)pyrazine (intermediate 9, 40.0 g, 136 mmol, 1 equiv) and XphosPdG3 (3.00 g, 3.50 mmol, 0.0250 equiv) in toluene (450 mL) under argon atmosphere. After complete addition, the reaction mixture is heated at 110° C. After 3 h, the reaction mixture is allowed to cool to room temperature. Water is added, and the mixture is filtered through a pad of Celite®. The filter cake is washed with toluene. The layers are separated, and the organic layer is washed with water and concentrated under reduced pressure. The residue is dissolved in a mixture of methanol (200 mL) and acetic acid (0.5 mL), and the mixture is stirred at room temperature. After 4 h, the mixture is filtered through a pad of Celite®, and the filtrate is concentrated under reduced pressure. The residue is purified by flash column chromatography (silica gel, gradient cyclohexane/EtOAc 80:20 to cyclohexane/EtOAc 60:40) to yield the desired product.

| | |
|---|---|
| C₁₂H₁₃N₃O₂ | (MW = 231.1 g/mol) |
| ESI | 232 [M + H]⁺ |
| Rₜ (HPLC) | 0.49 min (X011_S05) |

### Intermediate 11

### Ethyl 4-(3-(5-((4-methoxybenzyl)oxy)pyrazin-2-yl)thioureido)thiazole-5-carboxylate

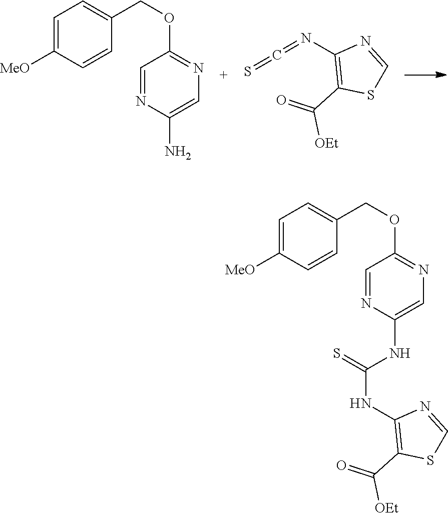 [SMILES: COc1ccc(COc2cnc(N)cn2)cc1 ; CCOC(=O)c1scnc1N=C=S ; CCOC(=O)c1scnc1NC(=S)Nc1cnc(OCc2ccc(OC)cc2)cn1]

5-((4-Methoxybenzyl)oxy)pyrazin-2-amine (intermediate 10, 7.19 g, 30.1 mmol, 1 equiv) is added to a solution of ethyl 4-isothiocyanatothiazole-5-carboxylate (intermediate 1, 6.46 g, 30.1 mmol, 1 equiv) in acetonitrile (70 mL), and the mixture is stirred at room temperature. After 18 h, the precipitate is filtered and dried to yield the desired product.

| | |
|---|---|
| C₁₉H₁₉N₅O₄S₂ | (MW = 445.1 g/mol) |
| ESI | 446 [M + H]⁺ |
| Rₜ (HPLC) | 1.15 min (Z018_S04) |

### Intermediate 12

### 6-(5-((4-Methoxybenzyl)oxy)pyrazin-2-yl)-5-thioxo-5,6-dihydrothiazolo[4,5-d]pyrimidin-7(4H)-one

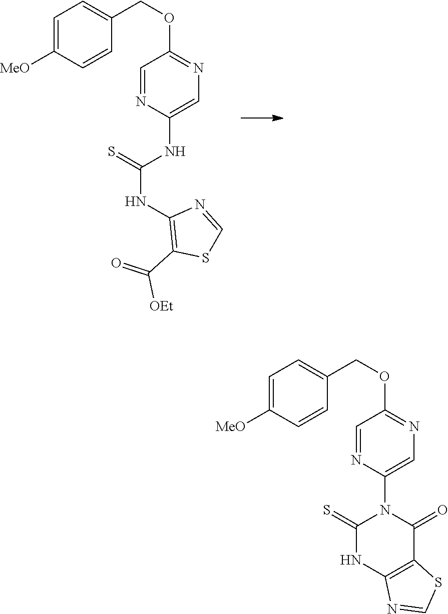 [SMILES: CCOC(=O)c1scnc1NC(=S)Nc1cnc(OCc2ccc(OC)cc2)cn1 ; COc1ccc(COc2cnc(-n3c(=S)[nH]c4ncsc4c3=O)cn2)cc1]

Ethyl 4-(3-(5-((4-methoxybenzyl)oxy)pyrazin-2-yl)thioureido)thiazole-5-carboxylate (intermediate 11, 16.0 g, 28.7 mmol, 1 equiv) is added in small portions to a mixture of aqueous sodium hydroxide solution (4 M, 28.7 mL, 113 mmol, 4.00 equiv) and methanol (160 mL), and the mixture is stirred at room temperature for 2 h. Water is added, and the mixture is neutralized with aqueous hydrochloric acid (4 M, 28.7 mL), and the precipitate is filtered and washed with water. The crude product is treated with a mixture of dichloromethane/methanol (400 mL, 95:5) and filtered, and the filtrate is concentrated under reduced pressure to yield the product.

| | |
|---|---|
| C₁₇H₁₃N₅O₃S₂ | (MW = 399.0 g/mol) |
| ESI | 400 [M + H]⁺ |
| Rₜ (HPLC) | 0.91 min (Z018_S04) |

### Intermediate 13 (General Procedure A)

### 6-(5-((4-Methoxybenzyl)oxy)pyrazin-2-yl)-5-((4-(trifluoromethyl)benzyl)thio)thiazolo[4,5-d]pyrimidin-7(6H)-one

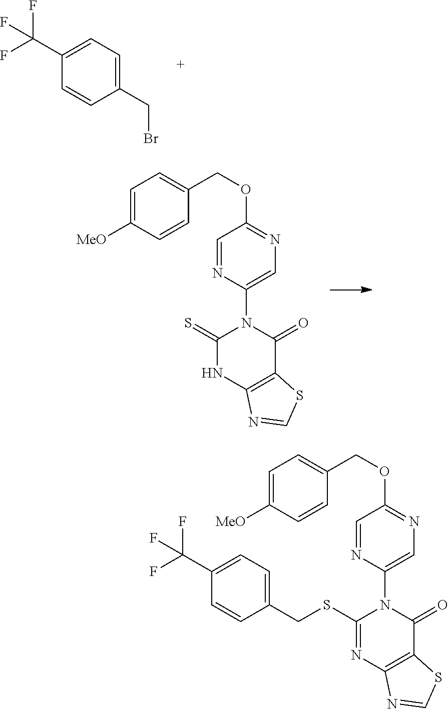

A solution of 1-(bromomethyl)-4-(trifluoromethyl)benzene (2.65 g, 10.9 mmol, 1 equiv) and N,N-diisopropylethylamine (6.00 mL, 33.6 mmol, 3.10 equiv) in propan-2-ol (10 mL) is added to a mixture of 6-(5-((4-methoxybenzyl)oxy)pyrazin-2-yl)-5-thioxo-5,6-dihydrothiazolo[4,5-d]pyrimidin-7(4H)-one (intermediate 12, 4.34 g, 10.9 mmol, 1 equiv) and propan-2-ol (70 mL). After 30 min water is added, and the precipitate is filtered and dried to yield the desired product.

| | |
|---|---|
| C₂₅H₁₈F₃N₅O₃S₂ | (MW = 557.1 g/mol) |
| ESI | 558 [M + H]⁺ |
| Rₜ (HPLC) | 1.13 min (Z018_S04) |

### Intermediate 14 (General Procedure B)

### 5-((4-(Difluoromethyl)benzyl)thio)-6-(5-((4-methoxybenzyl)oxy)pyrazin-2-yl)thiazolo[4,5-d]pyrimidin-7(6H)-one

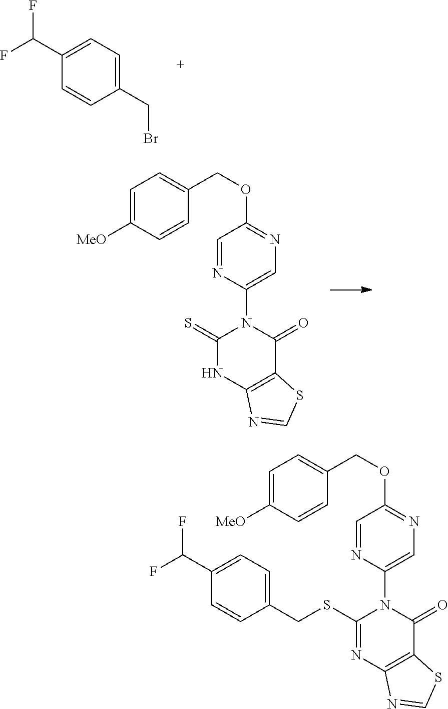

1-(Bromomethyl)-4-(difluoromethyl)benzene (277 mg, 1.25 mmol, 1 equiv) is added to a solution of 6-(5-((4-methoxybenzyl)oxy)pyrazin-2-yl)-5-thioxo-5,6-dihydrothiazolo[4,5-d]-pyrimidin-7(4H)-one (intermediate 12, 500 mg, 1.25 mmol, 1 equiv) and triethylamine (536 μL, 3.87 mmol, 3.10 equiv) in N,N-dimethylacetamide (3 mL). After 30 min, water is added, and the precipitate is filtered and dried to yield the desired product.

| | |
|---|---|
| C₂₅H₁₉F₂N₅O₃S₂ | (MW = 539.1 g/mol) |
| ESI | 540 [M + H]⁺ |
| Rₜ (HPLC) | 1.09 min (Z018_S04) |

The following intermediates are prepared using procedures analogous to the general procedures A and B described for intermediates 13 and 14, using appropriate starting materials. As is appreciated by those skilled in the art, the analogous examples may involve variations in general reaction conditions.

| | General | | | HPLC retention |
|---|---|---|---|---|
| Interme- | proce- | | | time [min] |
| diate | dure | Structure | ESI-MS | (method) |
| 15 | B | 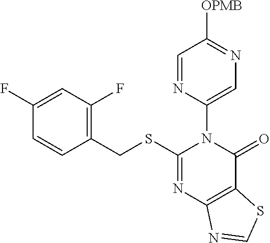 | 526 [M + H]⁺ | 1.08 (Z018_S04) |
| 16 | B | 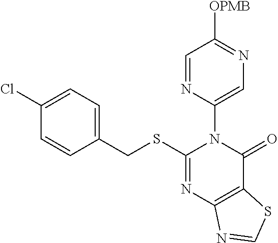 | 524/526 [M + H]⁺ | 1.10 (Z018_S04) |
| 17 | B | 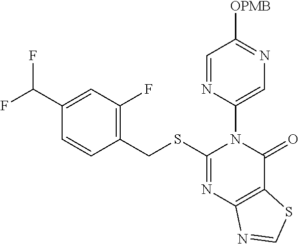 | 558 [M + H]⁺ | 1.09 (Z018_S04) |
| 18 | B | 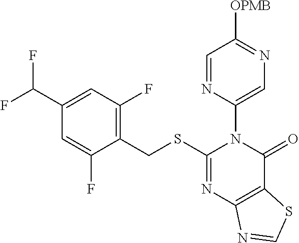 | 576 [M + H]⁺ | 1.11 (Z018_S04) |
| 19 | B | 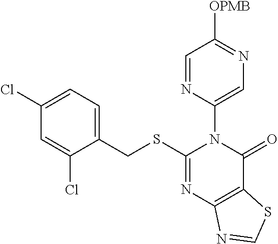 | 558/560 [M + H]⁺ | 1.19 (Z018_S04) |
| 20 | B | 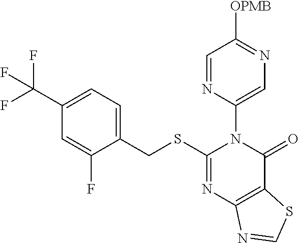 | 576 [M + H]⁺ | 1.16 (Z018_S04) |
| 21 | B | 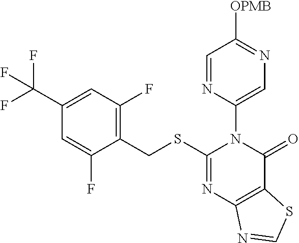 | 594 [M + H]⁺ | 1.16 (Z018_S04) |
| 22 | B | 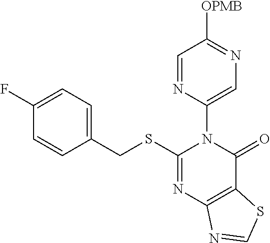 | 508 [M + H]⁺ | 1.10 (Z018_S04) |
| 23 | B | 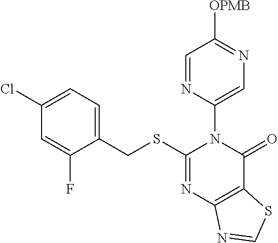 | 542/544 [M + H]⁺ | 1.15 (Z018_S04) |
| 24 | B | 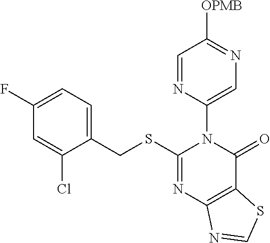 | 542/544 [M + H]⁺ | 1.14 (Z018_S04) |
| 25 | B | 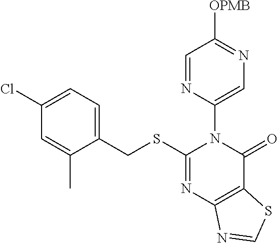 | 538/540 [M + H]⁺ | 1.17 (Z018_S04) |
| 26 | B | 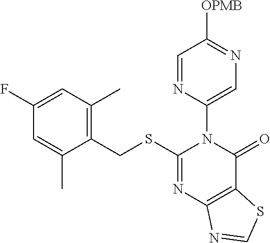 | 536 [M + H]⁺ | 1.16 (Z018_S04) |
| 27 | B | 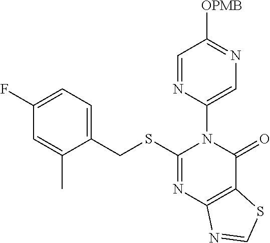 | 522 [M + H]⁺ | 1.13 (Z018_S04) |
| 28 | A | 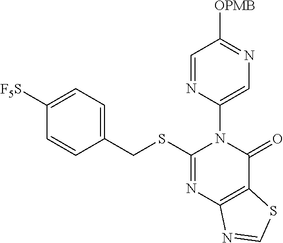 | 616 [M + H]⁺ | 0.80 (X012_S01) |
| 29 | A | 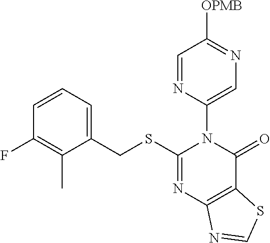 | 522 [M + H]⁺ | 0.75 (X012_S01) |
| 30 | A | 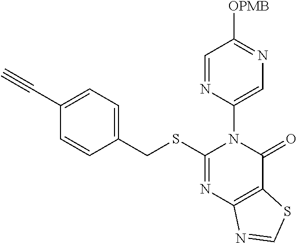 | 514 [M + H]⁺ | 0.75 (X012_S01) |
| 31 | B | 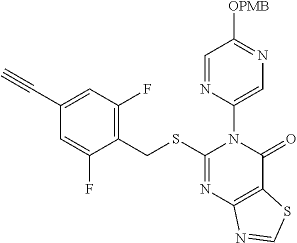 | 550 [M + H]⁺ | 1.09 (X018_S03) |

### Intermediate 32

### Ethyl 4-(3-(5-fluoro-6-hydroxypyridin-3-yl)thioureido)thiazole-5-carboxylate

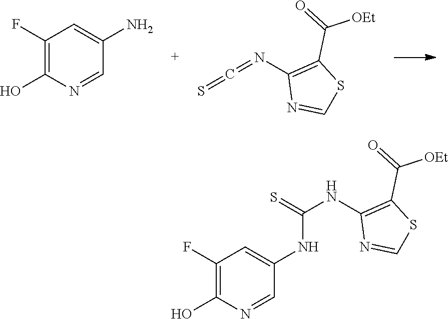 [SMILES: Nc1cnc(O)c(F)c1 ; CCOC(=O)c1scnc1N=C=S ; CCOC(=O)c1scnc1NC(=S)Nc1cnc(O)c(F)c1]

Ethyl 4-isothiocyanatothiazole-5-carboxylate (intermediate 1, 2.00 g. 9.37 mmol, 1 equiv) is added to a solution of 5-amino-3-fluoropyridin-2-ol (1.50 g, 9.37 mmol, 1 equiv) in 2-methyltetrahydrofuran (15 mL). After 18 h, tert-butylmethylether is added, and the precipitate is filtered to yield the product.

| | |
|---|---|
| C₁₂H₁₁FN₄O₃S₂ | (MW = 342.0 g/mol) |
| ESI | 343 [M + H]⁺ |
| Rₜ (HPLC) | 0.89 min (Z020_S01) |

### Intermediate 33

### 6-(5-Fluoro-6-hydroxypyridin-3-yl)-5-thioxo-5,6-dihydrothiazolo[4,5-d]pyrimidin-7(4H)one

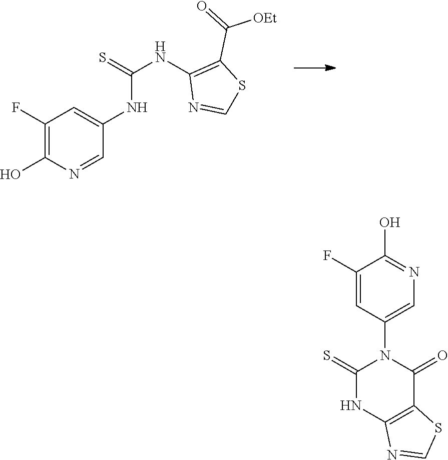 [SMILES: CCOC(=O)c1scnc1NC(=S)Nc1cnc(O)c(F)c1 ; O=c1c2scnc2[nH]c(=S)n1-c1cnc(O)c(F)c1]

Ethyl 4-(3-(5-fluoro-6-hydroxypyridin-3-yl)thioureido)thiazole-5-carboxylate (intermediate 32, 525 mg, 1.53 mmol, 1 equiv) is added in small portions to a solution of sodium hydroxide in water (0.1 M, 12 mL, 1.53 mmol, 1 equiv), and the mixture is stirred at room temperature. After 30 min, the reaction mixture is poured on aqueous potassium bisulfate solution, and the precipitate is filtered and washed with water to yield the product.

| | |
|---|---|
| C₁₀H₅FN₄O₂S₂ | (MW = 296.0 g/mol) |
| ESI | 297 [M + H]⁺ |
| Rₜ (HPLC) | 0.60 min (Z020_S01) |

### Intermediate 34

### 3-(5-((4-Methoxybenzyl)oxy)pyrazin-2-yl)-2-thioxo-2,3-dihydrothieno[3,2-d]pyrimidin-4(11H)-one

### Step 1

### Methyl 3-isothiocyanatothiophene-2-carboxylate

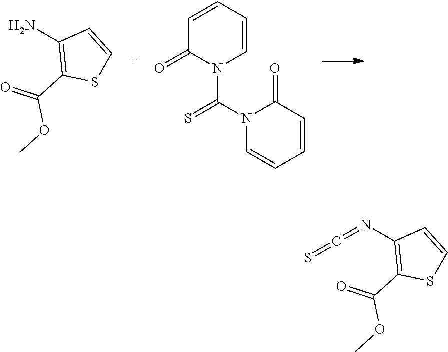

1,1′-Thiocarbonylbis(pyridin-2(1H)-one) (15.1 g, 64.8 mmol, 1.05 equiv) is added to a solution of methyl 3-aminothiophene-2-carboxylate (10.0 g, 61.7 mmol, 1 equiv) in acetonitrile (50 mL), and the reaction mixture is heated at 75° C. After 2 h, the reaction mixture is poured on ice-water, and the mixture is extracted with tert-butylmethylether. The phases are separated, and the organic phase is washed with water and dried over magnesium sulfate. All volatiles are removed under reduced pressure, and the residue is purified by flash column chromatography (silica gel, gradient cyclohexane to cyclohexane/EtOAc 70:30) to provide the product.

| | |
|---|---|
| C₇H₅NO₂S₂ | (MW = 199.2 g/mol) |
| ESI | not detected |
| Rₜ (HPLC) | 1.07 min (Z020_S01) |

### Step 2

### 3-(5-((4-Methoxybenzyl)oxy)pyrazin-2-yl)-2-thioxo-2,3-dihydrothieno[3,2-d]pyrimidin-4(11H)-one

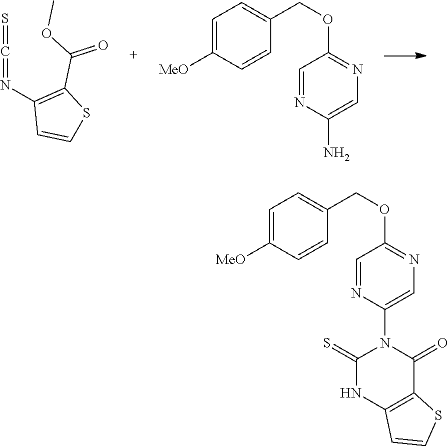 [SMILES: COC(=O)c1sccc1N=C=S ; COc1ccc(COc2cnc(N)cn2)cc1 ; COc1ccc(COc2cnc(-n3c(=S)[nH]c4ccsc4c3=O)cn2)cc1]

A mixture of 5-((4-Methoxybenzyl)oxy)pyrazin-2-amine (intermediate 10, 1.71 g, 7.15 mmol, 1 equiv) and methyl 3-isothiocyanatothiophene-2-carboxylate (step 1, 1.50 g, 7.15 mmol, 1 equiv) in acetontrile (50 mL) is stirred at room temperature over night and then heated at 50° C. for 1.5 h. Potassium tert-butoxide (802 mg, 7.15 mmol, 1 equiv) is added, and the mixture is stirred for 2 h and then cooled at room temperature. Water is added, and the mixture is neutralized with aqueous hydrochloric acid. The precipitate is filtered and dried. The residue is triturated with acetonitrile and dried to obtain the desired product.

| | |
|---|---|
| C₁₀H₅FN₄O₂S₂ | (MW = 398.1 g/mol) |
| ESI | 399 [M + H]⁺ |
| Rₜ (HPLC) | 0.57 min (X012_S01) |

### Intermediate 35

### Methyl 3-(3-(5-fluoro-6-hydroxypyridin-3-yl)thioureido)thiophene-2-carboxylate

### Step 1

### Methyl 3-isothiocyanatothiophene-2-carboxylate

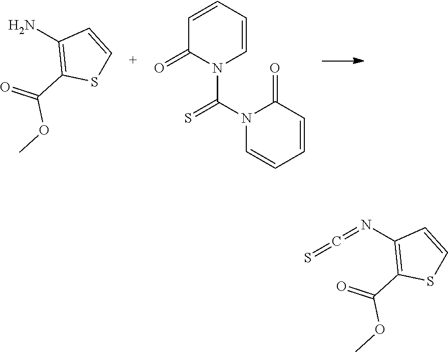

1,1′-Thiocarbonylbis(pyridin-2(1H)-one) (15.1 g, 64.8 mmol, 1.05 equiv) is added to a solution of methyl 3-aminothiophene-2-carboxylate (10.0 g, 61.7 mmol, 1 equiv) in acetonitrile (50 mL), and the reaction mixture is heated at 75° C. After 2 h, the reaction mixture is poured on ice-water, and the mixture is extracted with tert-butylmethylether. The phases are separated, and the organic phase is washed with water and dried over magnesium sulfate. All volatiles are removed under reduced pressure, and the residue is purified by flash column chromatography (silica gel, gradient cyclohexane to cyclohexane/EtOAc 70:30) to provide the product.

| | |
|---|---|
| C₇H₅NO₂S₂ | (MW = 199.2 g/mol) |
| ESI | not detected |
| Rₜ (HPLC) | 1.07 min (Z020_S01) |

### Step 2

### Methyl 3-(3-(5-fluoro-6-hydroxypyridin-3-yl)thioureido)thiophene-2-carboxylate

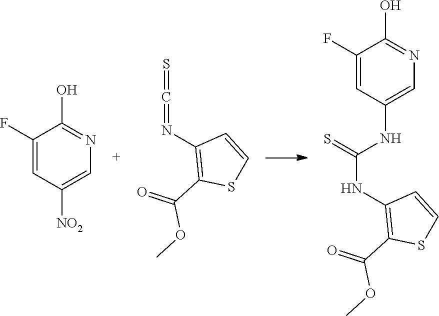 [SMILES: O=[N+]([O-])c1cnc(O)c(F)c1 ; COC(=O)c1sccc1N=C=S ; COC(=O)c1sccc1NC(=S)Nc1cnc(O)c(F)c1]

A mixture of 3-fluoro-5-nitropyridin-2-ol (500 mg, 3.10 mmol, 1 equiv), palladium on carbon (10%, 50 mg) and ammonia (2M in MeOH, 155 μL, 310 μmol, 0.100 equiv) in methanol (10 ml) is stirred under hydrogen atmosphere (3 bar). After 24 h, the mixture is filtered, 1-methyl-2-pyrolidinon (3 mL) is added to the filtrate and methanol is distilled off under reduced pressure. The residue is diluted with 2-methyltetrahydrofuran (3 mL) and methyl 3-isothiocyanatothiophene-2-carboxylate (step 1, 617 mg, 3.00 mmol, 1 equiv) is added. After 72 h, methyl tert-butyl ether is added, and the precipitate is filtered and dried to yield the desired product.

| | |
|---|---|
| C₁₂H₁₀FN₃O₃S₂ | (MW = 327.0 g/mol) |
| ESI | 328 [M + H]⁺ |
| Rₜ (HPLC) | 0.43 min (X012_S01) |

### Intermediate 36

### 3-(5-fluoro-6-hydroxypyridin-3-yl)-2-thioxo-2,3-dihydrothieno[3,2-d]pyrimidin-4(1H)-one

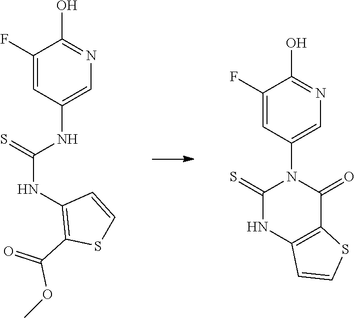 [SMILES: COC(=O)c1sccc1NC(=S)Nc1cnc(O)c(F)c1 ; O=c1c2sccc2[nH]c(=S)n1-c1cnc(O)c(F)c1]

A solution of sodium hydroxide in water (2M, 1.71 mL, 3.42 mmol, 2.00 equiv) is added to a mixture of methyl 3-(3-(5-fluoro-6-hydroxypyridin-3-yl)thioureido)thiophene-2-carboxylate (intermediate 35, 559 mg, 1.71 mmol, 1 equiv) in water (10 mL). After 30 min, the reaction mixture is acidified with hydrochloric acid (2M), and the precipitate is filtered and dried to yield the desired product.

| | |
|---|---|
| C₁₁H₆FN₃O₂S₂ | (MW = 295.0 g/mol) |
| ESI | 296 [M + H]⁺ |
| Rₜ (HPLC) | 0.30 min (X012_S01) |

### Intermediate 37

### 6-(5-((4-Methoxybenzyl)oxy)pyrazin-2-yl)-5-thioxo-5,6-dihydro-[1,2,3]thiadiazolo[4,5-d]-pyrimidin-7(4H)-one

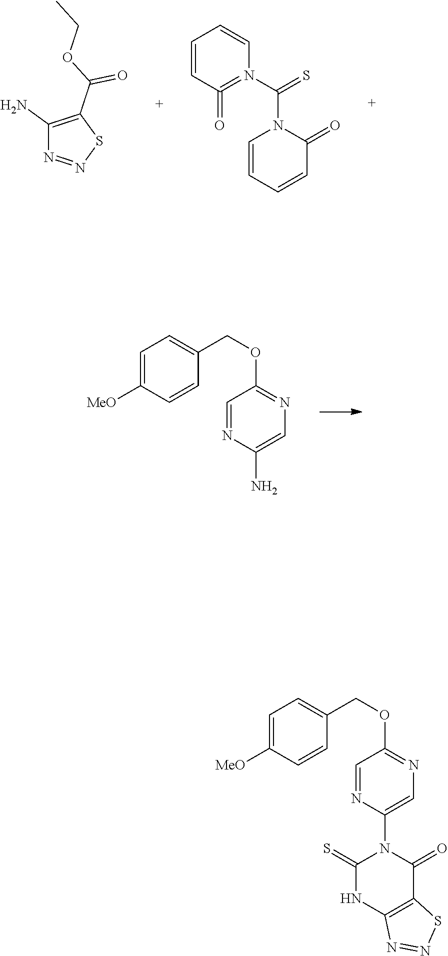

A mixture of ethyl 4-amino-1,2,3-thiadiazole-5-carboxylate (1.50 g, 8.23 mmol, 1 equiv) and 1,1′-thiocarbonylbis(pyridin-2(1H)-one) (2.21 g, 9.05 mmol, 1.10 equiv) in acetonitrile is heated at 70° C. After 1.5 h, 5-((4-methoxybenzyl)oxy)pyrazin-2-amine (intermediate 10, 2.00 g, 8.64 mmol, 1.10 equiv) is added. After 30 min, the reaction mixture is cooled at room temperature, and concentrated under reduced pressure. The residue is dissolved in methanol (40 mL), and potassium tert-butoxide is added. After 20 min, the reaction mixture is acidified with aqueous hydrochloric acid, and the mixture is extracted with ethyl acetate. The organic phase is washed with water and dried over magnesium sulfate. The dried solution is filtered, and the filtrate is concentrated under reduced pressure to yield the desired product.

| | |
|---|---|
| C₁₆H₁₂N₆O₃S₂ | (MW = 400.4 g/mol) |
| ESI | 401 [M + H]⁺ |
| Rₜ (HPLC) | 0.57 min (X012_S01) |

### Intermediate 38

### 6-(5-Fluoro-6-methoxypyridin-3-yl)-5-thioxo-5,6-dihydro-[1,2,3]thiadiazolo[4,5-d]pyrimidin-7(4H)-one

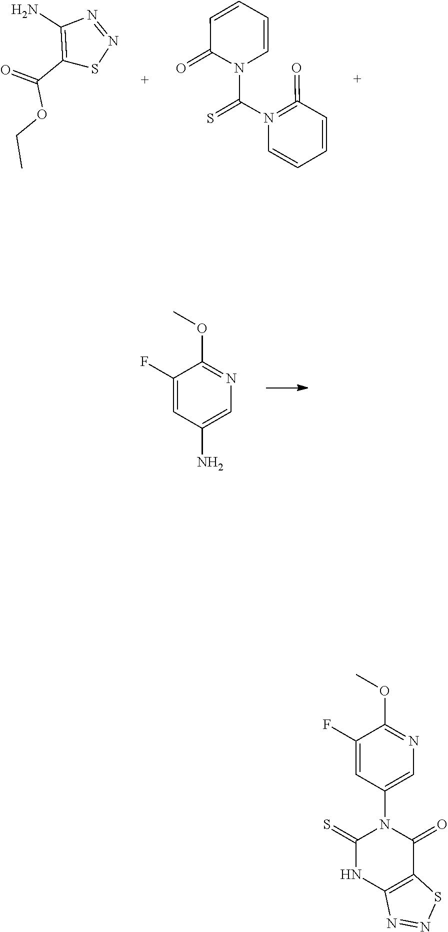

Intermediate 38 is prepared using procedures analogous to those described for intermediate 37, using appropriate starting materials.

| | |
|---|---|
| C₁₀H₆FN₅O₂S₂ | (MW = 311.3 g/mol) |
| ESI | 312 [M + H]⁺ |
| Rₜ (HPLC) | 0.45 min (X012_S01) |

### Preparation of Final Compounds

### Example 1

### 6-(6-Fluoro-5-hydroxypyrazin-2-yl)-5-((4-(trifluoromethyl)benzyl)thio)thiazolo[4,5-d]pyrimidin-7(6H)-one

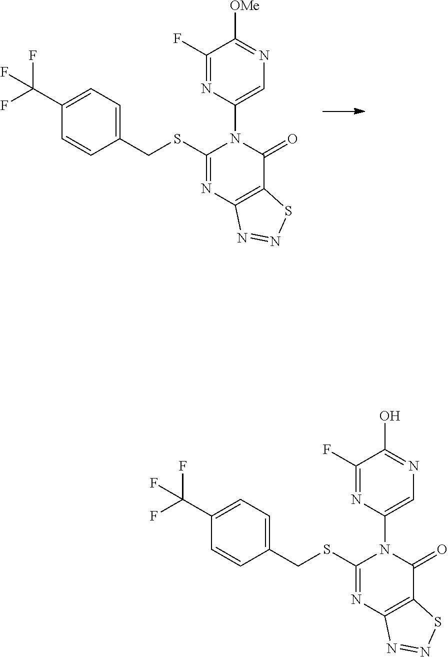 [SMILES: COc1ncc(-n2c(SCc3ccc(C(F)(F)F)cc3)nc3nnsc3c2=O)nc1F ; O=c1c2snnc2nc(SCc2ccc(C(F)(F)F)cc2)n1-c1cnc(O)c(F)n1]

Sodium iodide (45.0 mg, 300 μmol, 2.60 equiv) and trimethylsilyl chloride (100 μL, 788 μmol, 6.70 equiv) are added to a solution of 6-(6-fluoro-5-methoxypyrazin-2-yl)-5-((4-(trifluoromethyl)benzyl)thio)thiazolo[4,5-d]pyrimidin-7(6H)-one (intermediate 3, 55.0 mg, 117 μmol, 1 equiv) in acetonitrile (3 mL). After 18 h, additional trimethylsilyl chloride (30.0 μL, 236 μmol, 2.02 equiv) is added. After 1 h, the reaction mixture is filtered, and the filtrate is purified by reversed phase HPLC (Agilent Sunfire™-C18, gradient of acetonitrile in water, 0.1% TFA) to yield the desired product.

| | |
|---|---|
| C₁₇H₉F₄N₅O₂S₂ | (MW = 455.0 g/mol) |
| ESI | 456 [M + H]⁺ |
| Rₜ (HPLC) | 0.94 min (Z018_S04) |

The following examples are prepared using procedures analogous to those described for example 1, using appropriate starting materials. As is appreciated by those skilled in the art, the analogous examples may involve variations in general reaction conditions.

| | | | HPLC retention |
|---|---|---|---|
| Example | Structure | ESI-MS | time [min] (method) |
| 2 | 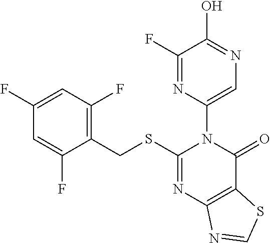 [SMILES: O=c1c2scnc2nc(SCc2c(F)cc(F)cc2F)n1-c1cnc(O)c(F)n1] | 442 [M + H]⁺ | 0.89 (Z018_S04) |
| 3 | 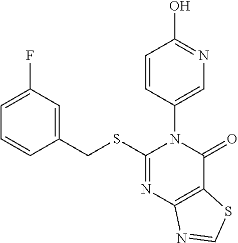 [SMILES: O=c1c2scnc2nc(SCc2cccc(F)c2)n1-c1ccc(O)nc1] | 387 [M + H]⁺ | 0.78 (Z018_S04) |
| 4 | 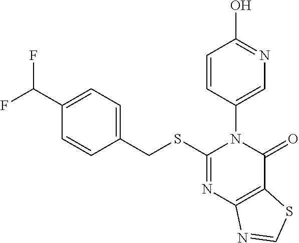 [SMILES: O=c1c2scnc2nc(SCc2ccc(C(F)F)cc2)n1-c1ccc(O)nc1] | 419 [M + H]⁺ | 0.80 (Z018_S04) |

### Example 3

### 5-((3-Fluorobenzyl)thio)-6-(6-hydroxypyridin-3-yl)thiazolo[4,5-d]pyrimidin-7(6H)-one

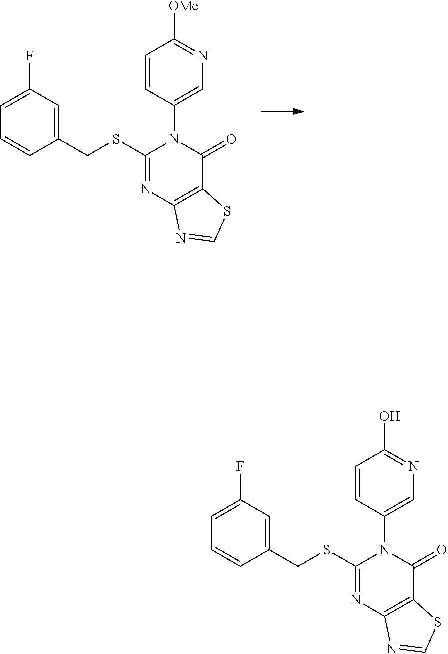 [SMILES: COc1ccc(-n2c(SCc3cccc(F)c3)nc3ncsc3c2=O)cn1 ; O=c1c2scnc2nc(SCc2cccc(F)c2)n1-c1ccc(O)nc1]

Potassium iodide (37.0 mg, 223 μmol, 0.900 equiv) and trimethylsilyl chloride (140 μL, 1.10 μmol, 4.60 equiv) are added to a solution of 5-((3-fluorobenzyl)thio)-6-(6-methoxypyridin-3-yl)thiazolo[4,5-d]pyrimidin-7(6H)-one (intermediate 5, 95.0 mg, 237 μmol, 1 equiv) in acetonitrile (2 mL). After 18 h, acetic acid and methanol are added, and the reaction mixture is purified by reversed phase HPLC (Agilent Sunfire™-C18, gradient of acetonitrile in water, 0.1% TFA) to yield the desired product.

| | |
|---|---|
| C₁₇H₁₁FN₄O₂S₂ | (MW = 386.0 g/mol) |
| ESI | 387 [M + H]⁺ |
| Rₜ (HPLC) | 0.78 (Z018_S04) |

The following examples are prepared using procedures analogous to those described for example 3, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 4 | 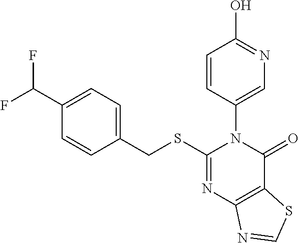 [SMILES: O=c1c2scnc2nc(SCc2ccc(C(F)F)cc2)n1-c1ccc(O)nc1] | 419 [M + H]⁺ | 0.80 (Z018_S04) |

### Example 5

### 6-(3-fluoro-5-hydroxypyrazin-2-yl)-5-((4-(trifluoromethyl)benzyl)thio)thiazolo[4,5-d]pyrimidin-7(6H)-one

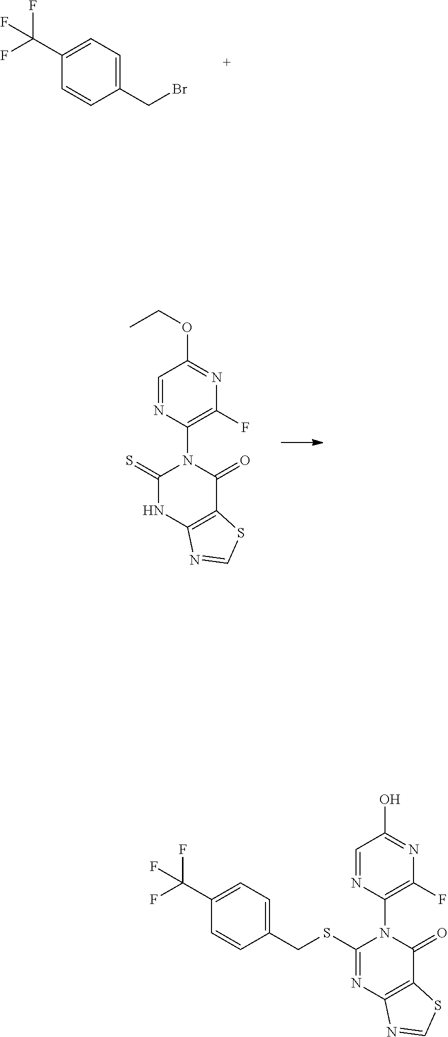 [SMILES: FC(F)(F)c1ccc(CBr)cc1 ; CCOc1cnc(-n2c(=S)[nH]c3ncsc3c2=O)c(F)n1 ; O=c1c2scnc2nc(SCc2ccc(C(F)(F)F)cc2)n1-c1ncc(O)nc1F]

1-(Bromomethyl)-4-(trifluoromethyl)benzene (6.75 mg, 28.0 μmol, 1 equiv) and triethylamine (10.0 μL, 72.0 μmol, 2.60 equiv) are added to a solution of 6-(5-ethoxy-3-fluoropyrazin-2-yl)-5-thioxo-5,6-dihydrothiazolo[4,5-d]pyrimidin-7(4H)-one (intermediate 8, 9.00 mg, 28.0 μmol, 1 equiv) in acetonitrile (3 mL). After 45 min, the reaction mixture is concentrated under reduced pressure. Pyridinium hydrochloride (9.59 mg, 83.0 μmol, 3.00 equiv) is added to the residue and the mixture is heated at 250° C. for 2 minutes with a heatgun. The mixture is cooled at room temperature and purified by reversed phase HPLC (Agilent Sunfire™-C18, gradient of acetonitrile in water, 0.1% TFA) to yield the desired product.

| | |
|---|---|
| C₁₇H₉F₄N₅O₂S₂ | (MW = 455.0 g/mol) |
| ESI | 456 [M + H]⁺ |
| Rₜ (HPLC) | 1.00 min (Z018_S04) |

### Example 6 (General Procedure C)

### 6-(5-Hydroxypyrazin-2-yl)-5-((4-(trifluoromethyl)benzyl)thio)thiazolo[4,5-d]pyrimidin-7(6H)-one

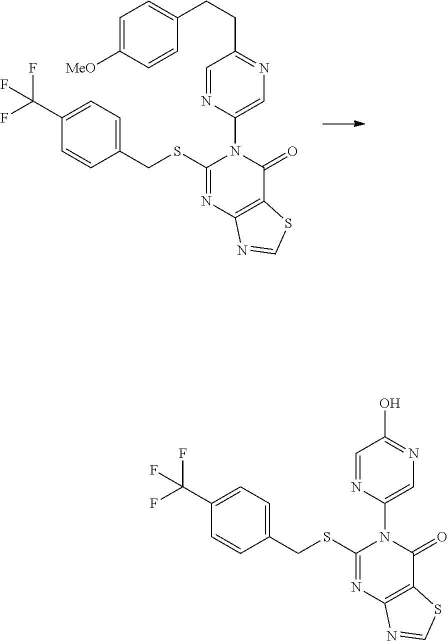 [SMILES: COc1ccc(CCc2cnc(-n3c(SCc4ccc(C(F)(F)F)cc4)nc4ncsc4c3=O)cn2)cc1 ; O=c1c2scnc2nc(SCc2ccc(C(F)(F)F)cc2)n1-c1cnc(O)cn1]

Amberlyst® 15(H) (58 mg, 267 μmol, 2.5 equiv) is added to a solution of 6-(5-((4-methoxybenzyl)oxy)pyrazin-2-yl)-5-((4-(trifluoromethyl)benzyl)thio)thiazolo[4,5-d]pyrimidin-7(6H)-one (intermediate 13, 58 mg, 104 μmol, 1 equiv) in methanol (1 mL), and the reaction mixture is heated at reflux. After 2.5 h, the reaction mixture is filtered, and the filtrate is cooled at room temperature and concentrated under reduced pressure. The residue is triturated with dichloromethane and dried to yield the product.

| | |
|---|---|
| C₁₇H₁₀F₃N₅O₂S₂ | (MW = 437.0 g/mol) |
| ESI | 436 [M − H]⁻ |
| Rₜ (HPLC) | 0.88 min (Z018_S04) |

### Example 7 (General Procedure D)

### 5-((4-(Difluoromethyl)benzyl)thio)-6-(5-hydroxypyrazin-2-yl)thiazolo[4,5-d]pyrimidin-7(6H)-one

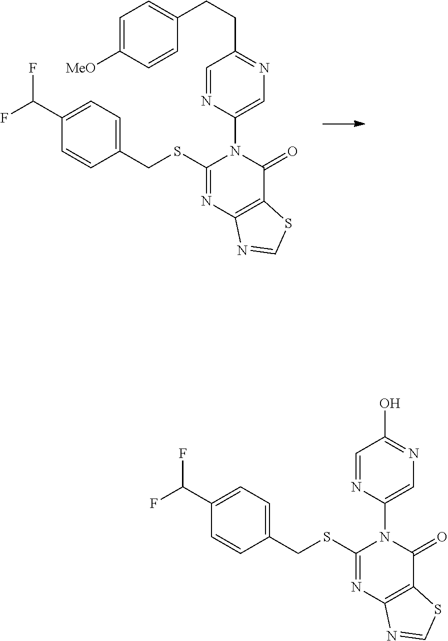 [SMILES: COc1ccc(CCc2cnc(-n3c(SCc4ccc(C(F)F)cc4)nc4ncsc4c3=O)cn2)cc1 ; O=c1c2scnc2nc(SCc2ccc(C(F)F)cc2)n1-c1cnc(O)cn1]

A mixture of 5-((4-(difluoromethyl)benzyl)thio)-6-(5-((4-methoxybenzyl)oxy)pyrazin-2-yl)thiazolo[4,5-d]pyrimidin-7(6H)-one (intermediate 14, 690 mg, 1.28 mmol, 1 equiv) and acetic acid (5 mL) is heated at 120° C. for 2 h. The mixture is concentrated under reduced pressure and the residue is purified by reversed phase HPLC (Gilson Sunfire™-C18, gradient of acetonitrile in water, 0.1% TFA) to yield the desired product.

| | |
|---|---|
| C₁₇H₁₁F₂N₅O₂S₂ | (MW = 419.0 g/mol) |
| ESI | 420 [M + H]⁺ |
| Rₜ (HPLC) | 0.81 min (Z018_S04) |

The following examples are prepared using procedures analogous to the general procedures C and D described for examples 6 and 7, using appropriate starting materials. As is appreciated by those skilled in the art, the analogous examples may involve variations in general reaction conditions.

| | General | | | HPLC retention |
|---|---|---|---|---|
| Example | procedure | Structure | ESI-MS | time [min] (method) |
| 8 | C | 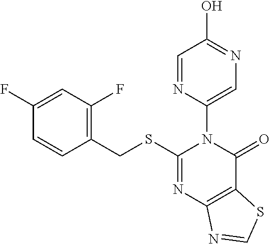 [SMILES: O=c1c2scnc2nc(SCc2ccc(F)cc2F)n1-c1cnc(O)cn1] | 406 [M + H]⁺ | 0.81 (Z018_S04) |
| 9 | C | 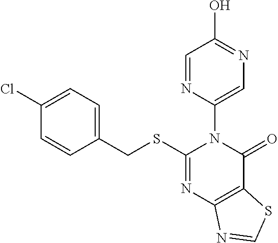 [SMILES: O=c1c2scnc2nc(SCc2ccc(Cl)cc2)n1-c1cnc(O)cn1] | 404 [M + H]⁺ | 0.84 (Z018_S04) |
| 10 | D | 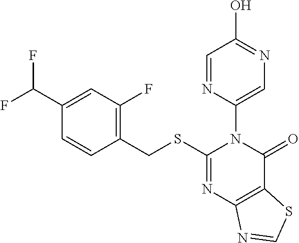 [SMILES: O=c1c2scnc2nc(SCc2ccc(C(F)F)cc2F)n1-c1cnc(O)cn1] | 438 [M + H]⁺ | 0.83 (Z018_S04) |
| 11 | D | 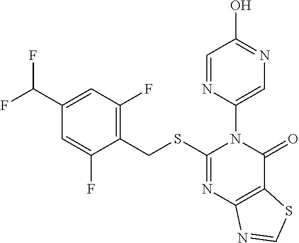 [SMILES: O=c1c2scnc2nc(SCc2c(F)cc(C(F)F)cc2F)n1-c1cnc(O)cn1] | 456 [M + H]⁺ | 0.84 (Z018_S04) |
| 12 | D | 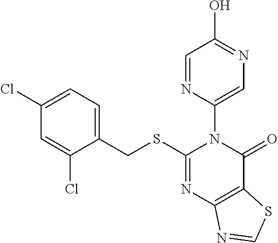 [SMILES: O=c1c2scnc2nc(SCc2ccc(Cl)cc2Cl)n1-c1cnc(O)cn1] | 438/440 [M + H]⁺ | 0.90 (Z018_S04) |
| 13 | D | 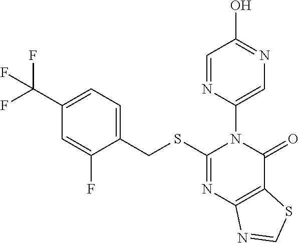 [SMILES: O=c1c2scnc2nc(SCc2ccc(C(F)(F)F)cc2F)n1-c1cnc(O)cn1] | 456 [M + H]⁺ | 0.89 (Z018_S04) |
| 14 | D | 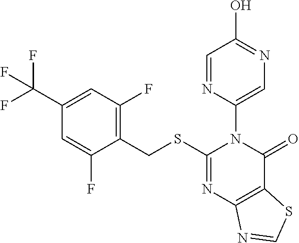 [SMILES: O=c1c2scnc2nc(SCc2c(F)cc(C(F)(F)F)cc2F)n1-c1cnc(O)cn1] | 474 [M + H]⁺ | 0.90 (Z018_S04) |
| 15 | D | 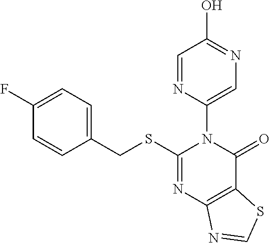 [SMILES: O=c1c2scnc2nc(SCc2ccc(F)cc2)n1-c1cnc(O)cn1] | 388 [M + H]⁺ | 0.78 (Z018_S04) |
| 16 | C | 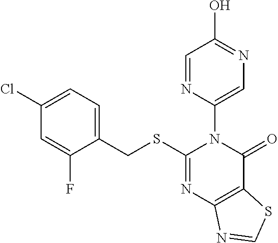 [SMILES: O=c1c2scnc2nc(SCc2ccc(Cl)cc2F)n1-c1cnc(O)cn1] | 422/424 [M + H]⁺ | 0.84 (Z018_S04) |
| 17 | D | 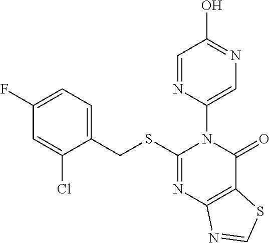 [SMILES: O=c1c2scnc2nc(SCc2ccc(F)cc2Cl)n1-c1cnc(O)cn1] | 422/424 [M + H]⁺ | 0.86 (Z018_S04) |
| 18 | C | 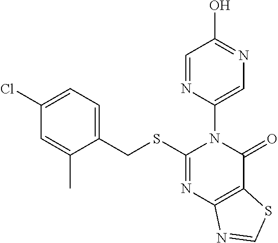 [SMILES: Cc1cc(Cl)ccc1CSc1nc2ncsc2c(=O)n1-c1cnc(O)cn1] | 418/420 [M + H]⁺ | 0.88 (Z018_S04) |
| 19 | C | 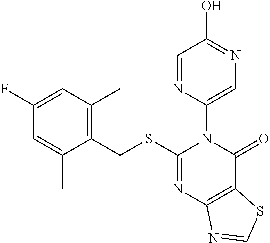 [SMILES: Cc1cc(F)cc(C)c1CSc1nc2ncsc2c(=O)n1-c1cnc(O)cn1] | 416 [M + H]⁺ | 0.88 (Z018_S04) |
| 20 | C | 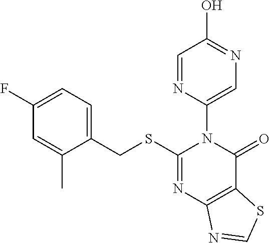 [SMILES: Cc1cc(F)ccc1CSc1nc2ncsc2c(=O)n1-c1cnc(O)cn1] | 402 [M + H]⁺ | 0.84 (Z018_S04) |
| 21 | C | 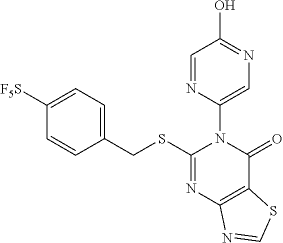 | 496 [M + H]⁺ | 0.56 (X012_S01) |
| 22 | C | 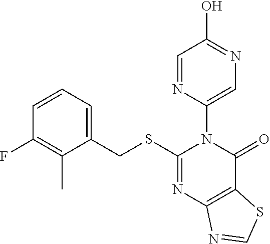 [SMILES: Cc1c(F)cccc1CSc1nc2ncsc2c(=O)n1-c1cnc(O)cn1] | 402 [M + H]⁺ | 0.49 (X012_S01) |
| 23 | C | 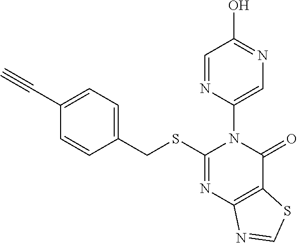 [SMILES: C#Cc1ccc(CSc2nc3ncsc3c(=O)n2-c2cnc(O)cn2)cc1] | 394 [M + H]⁺ | 0.47 (X012_S01) |
| 24 | C | 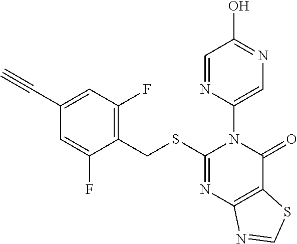 [SMILES: C#Cc1cc(F)c(CSc2nc3ncsc3c(=O)n2-c2cnc(O)cn2)c(F)c1] | 430 [M + H]⁺ | 0.70 (X018_S03) |

### Example 25

### 5-((2-Chloro-4,6-difluorobenzyl)thio)-6-(5-hydroxypyrazin-2-yl)thiazolo[4,5-d]pyrimidin-7(6H)-one

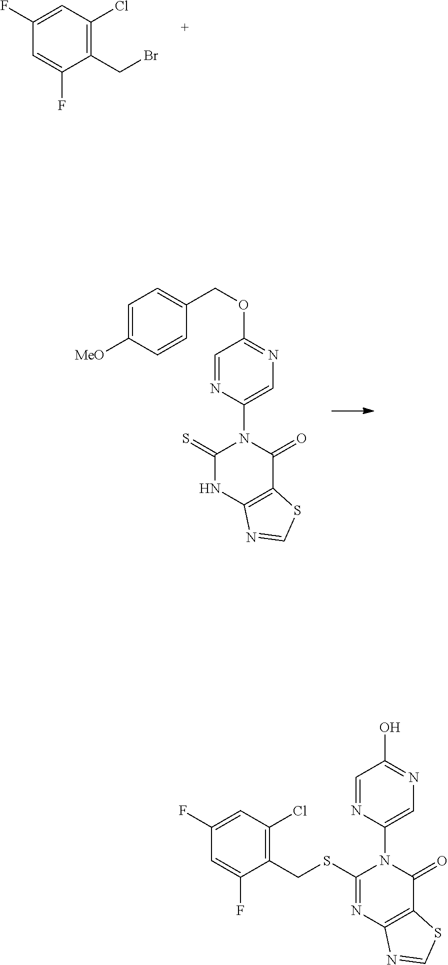 [SMILES: Fc1cc(F)c(CBr)c(Cl)c1 ; COc1ccc(COc2cnc(-n3c(=S)[nH]c4ncsc4c3=O)cn2)cc1 ; O=c1c2scnc2nc(SCc2c(F)cc(F)cc2Cl)n1-c1cnc(O)cn1]

2-(Bromomethyl)-1-chloro-3,5-difluorobenzene (21.2 mg, 88.0 μmol, 1 equiv) is added to a solution of 6-(5-((4-methoxybenzyl)oxy)pyrazin-2-yl)-5-thioxo-5,6-dihydrothiazolo[4,5-d]-pyrimidin-7(4H)-one (intermediate 12, 35 mg, 88.0 μmol, 1 equiv) and triethylamine (37.5 μL, 271 μmol, 3.10 equiv) in N,N-dimethylacetamide (2.5 mL). After 1 h, Amberlyst® 15(H) (95.2 mg, 438 mmol, 5.00 equiv) is added, and the reaction mixture is heated at 60° C. After 18 h, the reaction mixture is filtered, and the filtrate is purified by reversed phase HPLC (Waters XBridge™-C18, gradient of acetonitrile in water, 0.1% NH₃) to yield the desired product.

| | |
|---|---|
| C₁₆H₈ClF₂N₅O₂S₂ | (MW = 439.0 g/mol) |
| ESI | 440 [M + H]⁺ |
| Rₜ (HPLC) | 0.49 min (X012_S01) |

The following examples are prepared using procedures analogous to those described for example 25, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 26 | 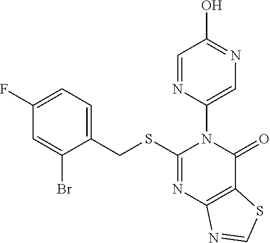 [SMILES: O=c1c2scnc2nc(SCc2ccc(F)cc2Br)n1-c1cnc(O)cn1] | 466 [M + H]⁺ | 0.72 (007_CA11) |
| 27 | 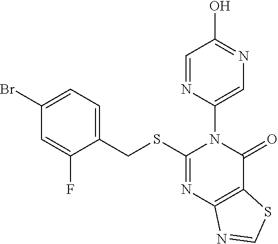 [SMILES: O=c1c2scnc2nc(SCc2ccc(Br)cc2F)n1-c1cnc(O)cn1] | 466 [M + H]⁺ | 0.76 (007_CA11) |
| 28 | 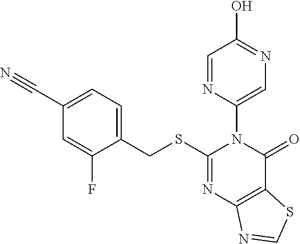 [SMILES: N#Cc1ccc(CSc2nc3ncsc3c(=O)n2-c2cnc(O)cn2)c(F)c1] | 413 [M + H]⁺ | 0.61 (007_CA02) |
| 29 | 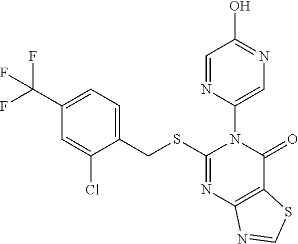 [SMILES: O=c1c2scnc2nc(SCc2ccc(C(F)(F)F)cc2Cl)n1-c1cnc(O)cn1] | 472 [M + H]⁺ | 0.44 (008_CA02) |
| 30 | 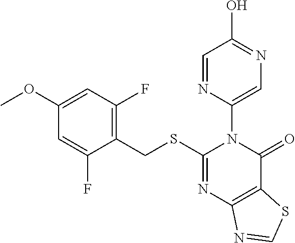 [SMILES: COc1cc(F)c(CSc2nc3ncsc3c(=O)n2-c2cnc(O)cn2)c(F)c1] | 436 [M + H]⁺ | 0.70 (007_CA02) |
| 31 | 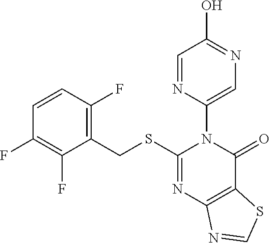 [SMILES: O=c1c2scnc2nc(SCc2c(F)ccc(F)c2F)n1-c1cnc(O)cn1] | 424 [M + H]⁺ | 0.67 (007_CA02) |
| 32 | 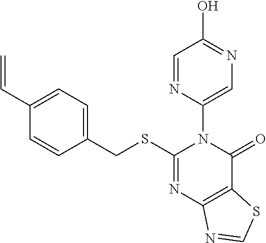 [SMILES: C=Cc1ccc(CSc2nc3ncsc3c(=O)n2-c2cnc(O)cn2)cc1] | 396 [M + H]⁺ | 0.73 (007_CA02) |
| 33 | 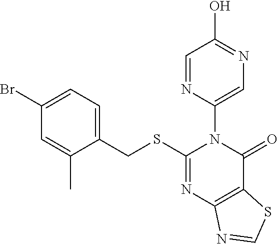 [SMILES: Cc1cc(Br)ccc1CSc1nc2ncsc2c(=O)n1-c1cnc(O)cn1] | 462 [M + H]⁺ | 0.41 (008_CA02) |
| 34 | 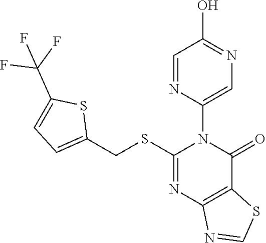 [SMILES: O=c1c2scnc2nc(SCc2ccc(C(F)(F)F)s2)n1-c1cnc(O)cn1] | 444 [M + H]⁺ | 0.76 (007_CA02) |
| 35 | 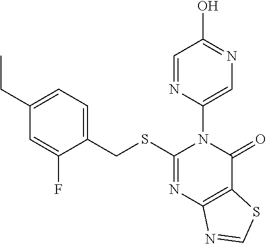 [SMILES: CCc1ccc(CSc2nc3ncsc3c(=O)n2-c2cnc(O)cn2)c(F)c1] | 416 [M + H]⁺ | 0.79 (007_CA02) |
| 36 | 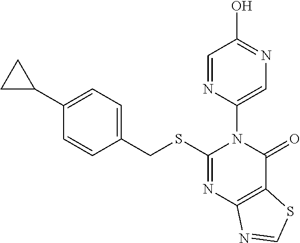 [SMILES: O=c1c2scnc2nc(SCc2ccc(C3CC3)cc2)n1-c1cnc(O)cn1] | 410 [M + H]⁺ | 0.40 (008_CA02) |
| 37 | 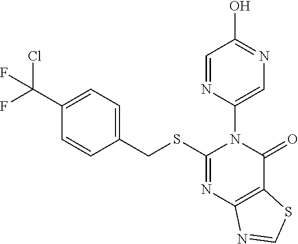 [SMILES: O=c1c2scnc2nc(SCc2ccc(C(F)(F)Cl)cc2)n1-c1cnc(O)cn1] | 454 [M + H]⁺ | 0.81 (007_CA02) |
| 38 | 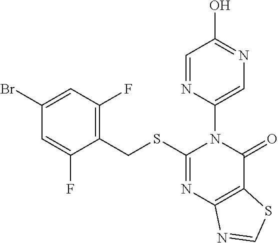 [SMILES: O=c1c2scnc2nc(SCc2c(F)cc(Br)cc2F)n1-c1cnc(O)cn1] | 484 [M + H]⁺ | 0.76 (007_CA02) |
| 39 | 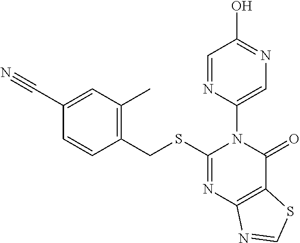 [SMILES: Cc1cc(C#N)ccc1CSc1nc2ncsc2c(=O)n1-c1cnc(O)cn1] | 409 [M + H]⁺ | 0.62 (007_CA11) |
| 40 | 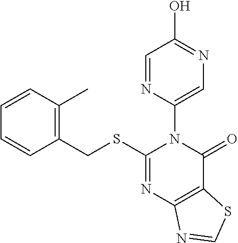 [SMILES: Cc1ccccc1CSc1nc2ncsc2c(=O)n1-c1cnc(O)cn1] | 384 [M + H]⁺ | 0.67 (007_CA11) |
| 41 | 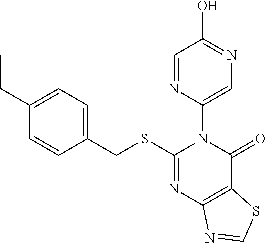 [SMILES: CCc1ccc(CSc2nc3ncsc3c(=O)n2-c2cnc(O)cn2)cc1] | 398 [M + H]⁺ | 0.75 (007_CA11) |
| 42 | 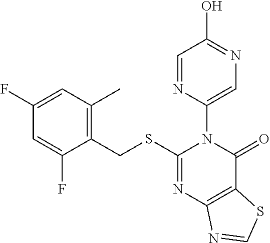 [SMILES: Cc1cc(F)cc(F)c1CSc1nc2ncsc2c(=O)n1-c1cnc(O)cn1] | 420 [M + H]⁺ | 0.72 (007_CA02) |
| 43 | 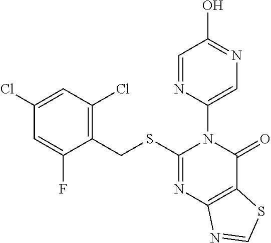 [SMILES: O=c1c2scnc2nc(SCc2c(F)cc(Cl)cc2Cl)n1-c1cnc(O)cn1] | 456 [M + H]⁺ | 0.79 (007_CA02) |
| 44 | 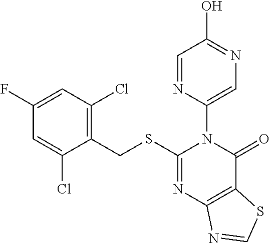 [SMILES: O=c1c2scnc2nc(SCc2c(Cl)cc(F)cc2Cl)n1-c1cnc(O)cn1] | 456 [M + H]⁺ | 0.77 (007_CA02) |
| 45 | 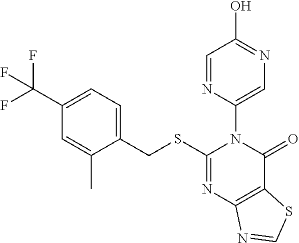 [SMILES: Cc1cc(C(F)(F)F)ccc1CSc1nc2ncsc2c(=O)n1-c1cnc(O)cn1] | 452 [M + H]⁺ | 0.82 (007_CA02) |
| 46 | 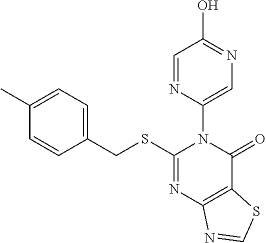 [SMILES: Cc1ccc(CSc2nc3ncsc3c(=O)n2-c2cnc(O)cn2)cc1] | 384 [M + H]⁺ | 0.71 (007_CA02) |
| 47 | 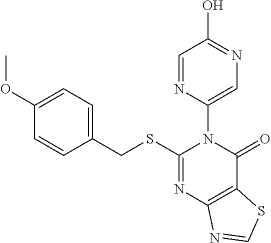 | 400 [M + H]⁺ | 0.44 (X012_S01) |
| 48 | 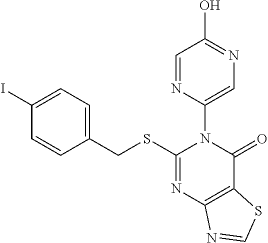 [SMILES: O=c1c2scnc2nc(SCc2ccc(I)cc2)n1-c1cnc(O)cn1] | 496 [M + H]⁺ | 0.77 (007_CA02) |
| 49 | 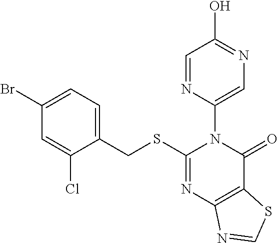 [SMILES: O=c1c2scnc2nc(SCc2ccc(Br)cc2Cl)n1-c1cnc(O)cn1] | 482 [M + H]⁺ | 0.81 (007_CA02) |
| 50 | 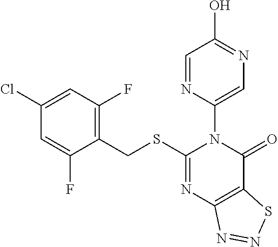 [SMILES: O=c1c2snnc2nc(SCc2c(F)cc(Cl)cc2F)n1-c1cnc(O)cn1] | 440 [M + H]⁺ | 0.50 (X012_S01) |
| 51 | 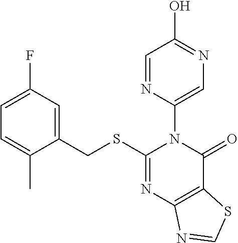 [SMILES: Cc1ccc(F)cc1CSc1nc2ncsc2c(=O)n1-c1cnc(O)cn1] | 402 [M + H]⁺ | 0.50 (X012_S01) |
| 52 | 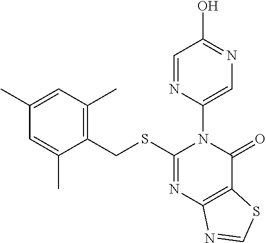 [SMILES: Cc1cc(C)c(CSc2nc3ncsc3c(=O)n2-c2cnc(O)cn2)c(C)c1] | 412 [M + H]⁺ | 0.82 (007_CA02) |
| 53 | 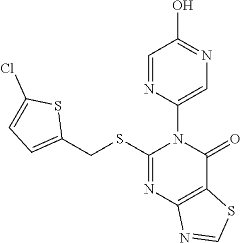 [SMILES: O=c1c2scnc2nc(SCc2ccc(Cl)s2)n1-c1cnc(O)cn1] | 410 [M + H]⁺ | 0.48 (X012_S01) |
| 54 | 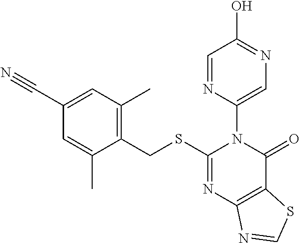 [SMILES: Cc1cc(C#N)cc(C)c1CSc1nc2ncsc2c(=O)n1-c1cnc(O)cn1] | 423 [M + H]⁺ | 0.38 (X011_S03) |
| 55 | 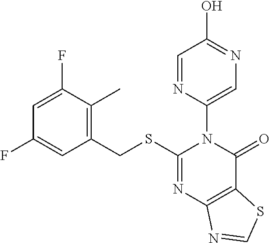 [SMILES: Cc1c(F)cc(F)cc1CSc1nc2ncsc2c(=O)n1-c1cnc(O)cn1] | 420 [M + H]⁺ | 0.52 (X012_S01) |
| 56 | 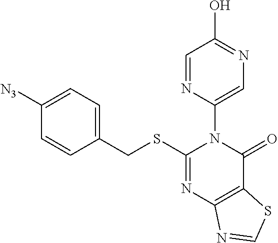 [SMILES: [N-]=[N+]=Nc1ccc(CSc2nc3ncsc3c(=O)n2-c2cnc(O)cn2)cc1] | 411 [M + H]⁺ | 0.49 (X012_S01) |
| 57 | 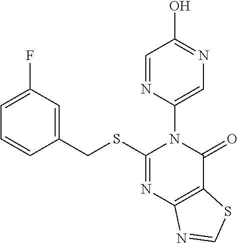 [SMILES: O=c1c2scnc2nc(SCc2cccc(F)c2)n1-c1cnc(O)cn1] | 388 [M + H]⁺ | 0.86 (Z018_S04) |

### Example 58

### 5-((4-(Difluoromethyl)-2,6-difluorobenzyl)thio)-6-(5-fluoro-6-hydroxypyridin-3-yl)thiazolo[4,5-d]pyrimidin-7(6H)-one

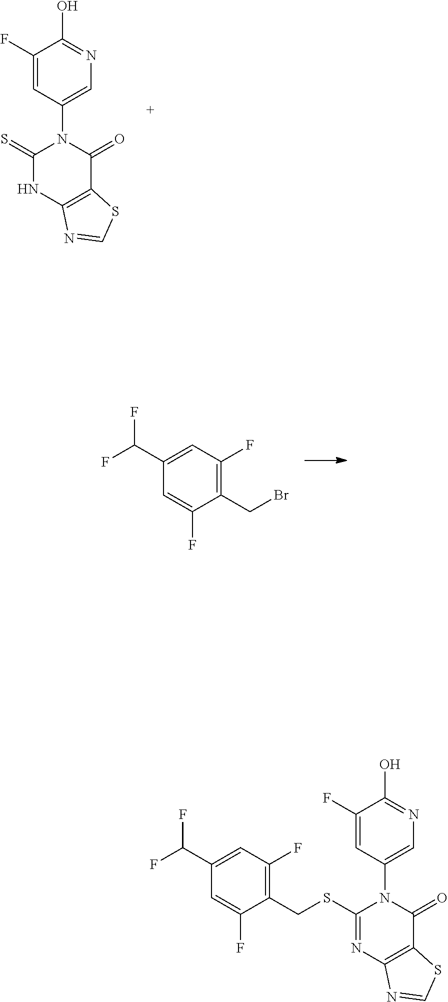 [SMILES: O=c1c2scnc2[nH]c(=S)n1-c1cnc(O)c(F)c1 ; Fc1cc(C(F)F)cc(F)c1CBr ; O=c1c2scnc2nc(SCc2c(F)cc(C(F)F)cc2F)n1-c1cnc(O)c(F)c1]

2-(Bromomethyl)-5-(difluoromethyl)-1,3-difluorobenzene (187 mg, 729 μmol, 0.900 equiv) is added to a solution of 6-(5-fluoro-6-hydroxypyridin-3-yl)-5-thioxo-5,6-dihydrothiazolo[4,5-d]pyrimidin-7(4H)-one (intermediate 33, 300 mg, 810 μmol, 1 equiv) and triethylamine (337 μL, 2.43 mmol, 3.00 equiv) in N,N-dimethylformamide (7 mL). After 1 h, the reaction mixture is filtered, and the filtrate is purified by reversed phase HPLC (Gilson XBridge™-C18, gradient of acetonitrile in water, 0.1% TFA) to yield the desired product.

| | |
|---|---|
| C₁₈H₉F₅N₄O₂S₂ | (MW = 472.0 g/mol) |
| ESI | 473 [M + H]⁺ |
| Rₜ (HPLC) | 0.50 min (X012_S01) |

The following examples are prepared using procedures analogous to those described for example 58, using appropriate starting materials. As is appreciated by those skilled in the art, the analogous examples may involve variations in general reaction conditions.

| | | | HPLC retention time [min] |
|---|---|---|---|
| Example | Structure | ESI-MS | (method) |
| 59 | 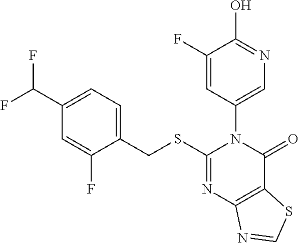 [SMILES: O=c1c2scnc2nc(SCc2ccc(C(F)F)cc2F)n1-c1cnc(O)c(F)c1] | 455 [M + H]⁺ | 0.49 (X012_S01) |
| 60 | 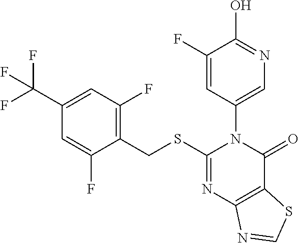 [SMILES: O=c1c2scnc2nc(SCc2c(F)cc(C(F)(F)F)cc2F)n1-c1cnc(O)c(F)c1] | 491 [M + H]⁺ | 0.54 (X012_S01) |
| 61 | 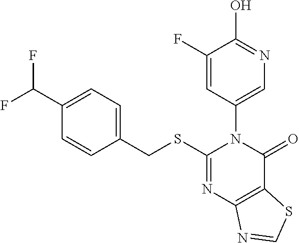 [SMILES: O=c1c2scnc2nc(SCc2ccc(C(F)F)cc2)n1-c1cnc(O)c(F)c1] | 437 [M + H]⁺ | 0.48 (X012_S01) |
| 62 | 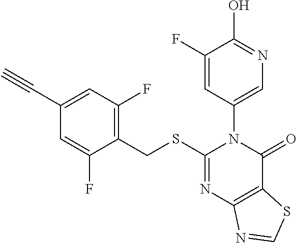 [SMILES: C#Cc1cc(F)c(CSc2nc3ncsc3c(=O)n2-c2cnc(O)c(F)c2)c(F)c1] | 447 [M + H]⁺ | 0.40 (X011_S04) |
| 63 | 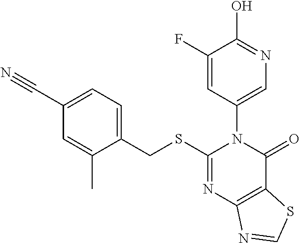 [SMILES: Cc1cc(C#N)ccc1CSc1nc2ncsc2c(=O)n1-c1cnc(O)c(F)c1] | 426 [M + H]⁺ | 0.89 (Z018_S04) |
| 64 | 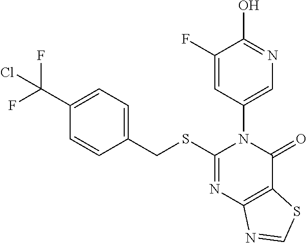 [SMILES: O=c1c2scnc2nc(SCc2ccc(C(F)(F)Cl)cc2)n1-c1cnc(O)c(F)c1] | 471 [M + H]⁺ | 0.56 (X012_S01) |
| 65 | 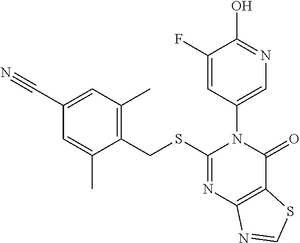 [SMILES: Cc1cc(C#N)cc(C)c1CSc1nc2ncsc2c(=O)n1-c1cnc(O)c(F)c1] | 440 [M + H]⁺ | 0.71 (007_CA02) |
| 66 | 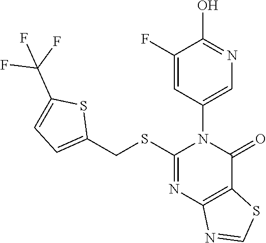 [SMILES: O=c1c2scnc2nc(SCc2ccc(C(F)(F)F)s2)n1-c1cnc(O)c(F)c1] | 461 [M + H]⁺ | 0.53 (X012_S01) |
| 67 | 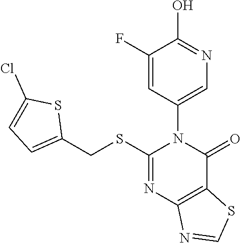 [SMILES: O=c1c2scnc2nc(SCc2ccc(Cl)s2)n1-c1cnc(O)c(F)c1] | 427 [M + H]⁺ | 0.94 (Z018_S04) |
| 68 | 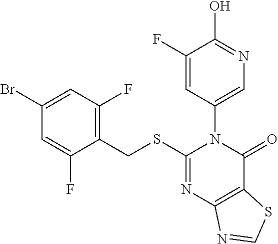 [SMILES: O=c1c2scnc2nc(SCc2c(F)cc(Br)cc2F)n1-c1cnc(O)c(F)c1] | 501 [M + H]⁺ | 0.77 (X018_S03) |
| 69 | 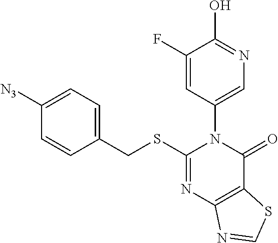 [SMILES: [N-]=[N+]=Nc1ccc(CSc2nc3ncsc3c(=O)n2-c2cnc(O)c(F)c2)cc1] | 428 [M + H]⁺ | 0.50 (X012_S01) |

### Example 70

### 2-((4-Chlorobenzyl)thio)-3-(5-hydroxypyrazin-2-yl)thieno[3,2-d]pyrimidin-4(3H)-one

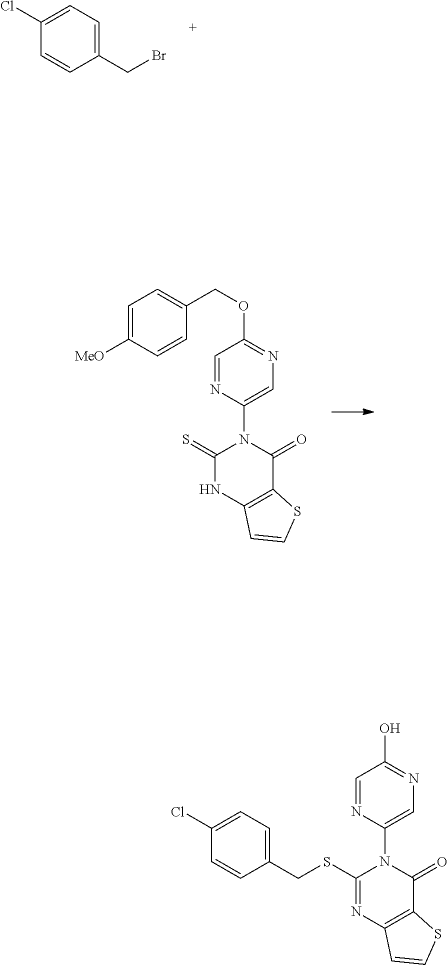 [SMILES: Clc1ccc(CBr)cc1 ; COc1ccc(COc2cnc(-n3c(=S)[nH]c4ccsc4c3=O)cn2)cc1 ; O=c1c2sccc2nc(SCc2ccc(Cl)cc2)n1-c1cnc(O)cn1]

1-(Bromomethyl)-4-chlorobenzene (27.0 mg, 127 μmol, 1 equiv) is added to a solution of 3-(5-((4-methoxybenzyl)oxy)pyrazin-2-yl)-2-thioxo-2,3-dihydrothieno[3,2-d]pyrimidin-4-(1H)-one (intermediate 34, 50 mg, 125 μmol, 1 equiv) and triethylamine (54.0 μL, 390 μmol, 3.10 equiv) in N,N-dimethylacetamide (2 mL). After 16 h, Amberlyst® 15(H) (274 mg, 1.26 μmol, 10.0 equiv) and methanol (0.5 mL) are added, and the reaction mixture is heated at 80° C. After 18 h, the reaction mixture is filtered, and the filtrate is purified by reversed phase HPLC (Waters XBridge™-C18, gradient of acetonitrile in water, 0.1% NH₃) to yield the desired product.

| | |
|---|---|
| C₁₇H₁₁ClN₄O₂S₂ | (MW = 402.0 g/mol) |
| ESI | 403 [M + H]⁺ |
| Rₜ (HPLC) | 0.58 min (X012_S01) |

The following examples are prepared using procedures analogous to those described for example 70, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 71 | 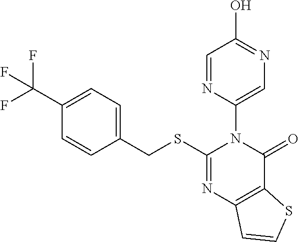 [SMILES: O=c1c2sccc2nc(SCc2ccc(C(F)(F)F)cc2)n1-c1cnc(O)cn1] | 437 [M + H]⁺ | 0.96 (Z018_S04) |
| 72 | 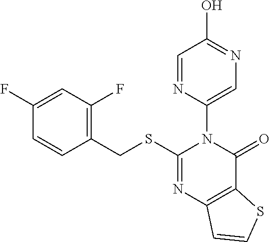 [SMILES: O=c1c2sccc2nc(SCc2ccc(F)cc2F)n1-c1cnc(O)cn1] | 405 [M + H]⁺ | 0.81 (007_CA11) |
| 73 | 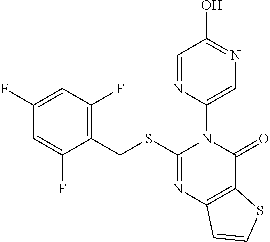 [SMILES: O=c1c2sccc2nc(SCc2c(F)cc(F)cc2F)n1-c1cnc(O)cn1] | 423 [M + H]⁺ | 0.82 (007_CA11) |
| 74 | 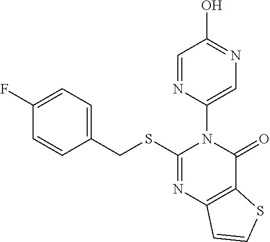 [SMILES: O=c1c2sccc2nc(SCc2ccc(F)cc2)n1-c1cnc(O)cn1] | 387 [M + H]⁺ | 0.79 (007_CA11) |
| 75 | 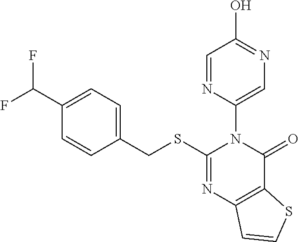 [SMILES: O=c1c2sccc2nc(SCc2ccc(C(F)F)cc2)n1-c1cnc(O)cn1] | 419 [M + H]⁺ | 0.80 (007_CA11) |
| 76 | 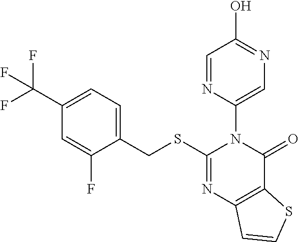 [SMILES: O=c1c2sccc2nc(SCc2ccc(C(F)(F)F)cc2F)n1-c1cnc(O)cn1] | 455 [M + H]⁺ | 0.90 (007_CA11) |

### Example 77

### 3-(5-Fluoro-6-hydroxypyridin-3-yl)-2-((2,4,6-trifluorobenzyl)thio)thieno[3,2-d]pyrimidin-4(31H)-one

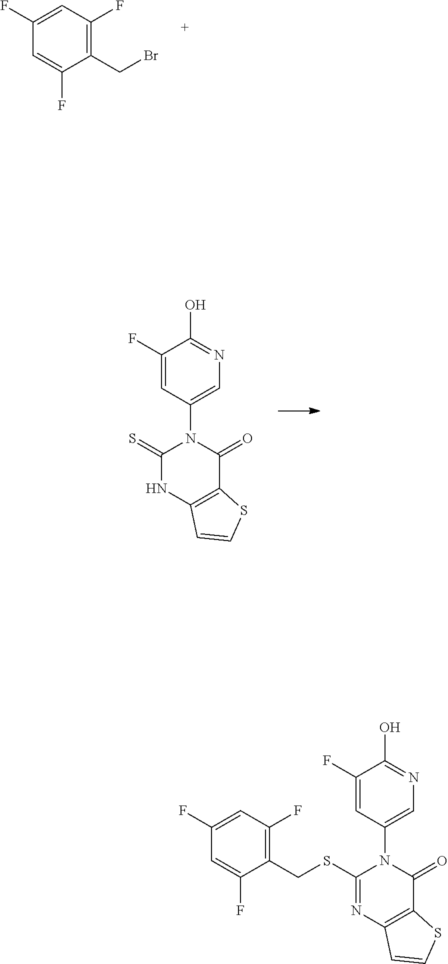 [SMILES: Fc1cc(F)c(CBr)c(F)c1 ; O=c1c2sccc2[nH]c(=S)n1-c1cnc(O)c(F)c1 ; O=c1c2sccc2nc(SCc2c(F)cc(F)cc2F)n1-c1cnc(O)c(F)c1]

Example 77 is prepared using procedures analogous to those described for example 58, using appropriate starting materials.

| | |
|---|---|
| C₁₈H₉F₄N₃O₂S₂ | (MW = 439.0 g/mol) |
| ESI | 440 [M + H]⁺ |
| Rₜ (HPLC) | 0.57 min (X012_S01) |

### Example 78

### 5-((4-Chlorobenzyl)thio)-6-(5-hydroxypyrazin-2-yl)-[1,2,3]thiadiazolo[4,5-d]pyrimidin-7(6H)-one

### Step 1

### 5-((4-Chlorobenzyl)thio)-6-(5-((4-methoxybenzyl)oxy)pyrazin-2-yl)-[1,2,3]thiadiazolo[4,5-d]pyrimidin-7(6H)-one

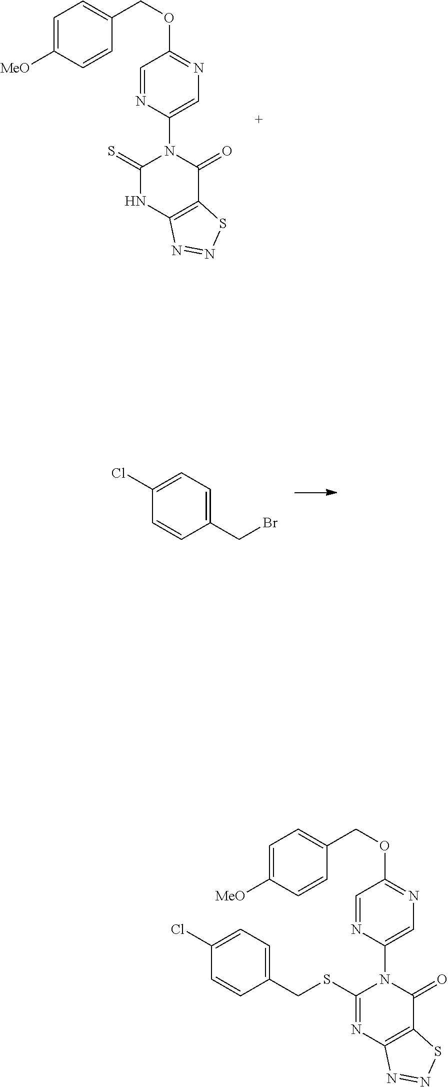 [SMILES: COc1ccc(COc2cnc(-n3c(=S)[nH]c4nnsc4c3=O)cn2)cc1 ; Clc1ccc(CBr)cc1 ; COc1ccc(COc2cnc(-n3c(SCc4ccc(Cl)cc4)nc4nnsc4c3=O)cn2)cc1]

1-(Bromomethyl)-4-chlorobenzene (89.0 mg, 425 μmol, 1 equiv) is added to a solution of 6-(5-((4-methoxybenzyl)oxy)pyrazin-2-yl)-5-thioxo-5,6-dihydro-[1,2,3]thiadiazolo[4,5-d]-pyrimidin-7(4H)-one (intermediate 37, 200 mg, 425 μmol, 1 equiv) and triethylamine (178 μL, 1.27 mmol, 3.00 equiv) in N,N-dimethylacetamide (2 mL). After 2 h, water and ethyl acetate are added, the layers are separated, and the aqueous layer is extracted with ethyl acetate. The combined organic layers are dried over magnesium sulfate, filtered, and concentrated under reduced pressure to yield the desired product.

### Step 2

### 5-((4-Chlorobenzyl)thio)-6-(5-hydroxypyrazin-2-yl)-[1,2,3]thiadiazolo[4,5-d]pyrimidin-7(6H)-one

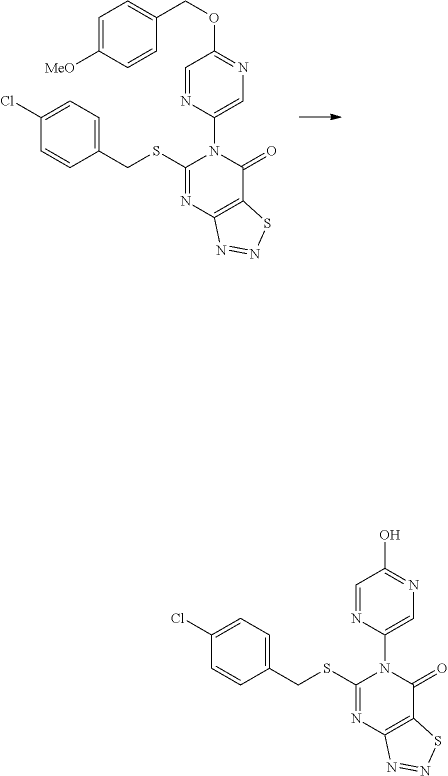 [SMILES: COc1ccc(COc2cnc(-n3c(SCc4ccc(Cl)cc4)nc4nnsc4c3=O)cn2)cc1 ; O=c1c2snnc2nc(SCc2ccc(Cl)cc2)n1-c1cnc(O)cn1]

A mixture of 5-((4-chlorobenzyl)thio)-6-(5-((4-methoxybenzyl)oxy)pyrazin-2-yl)-[1,2,3]-thiadiazolo[4,5-d]pyrimidin-7(6H)-one (step 1, 228 mg, 434 μmol, 1 equiv) and acetic acid (2 mL) is heated at 120° C. for 2 h. The mixture is concentrated under reduced pressure and the residue is purified by reversed phase HPLC (Waters XBridge™-C18, gradient of acetonitrile in water, 0.1% TFA) to yield the desired product.

| | |
|---|---|
| C₁₅H₉ClN₆O₂S₂ | (MW = 404.0 g/mol) |
| ESI | 405 [M + H]⁺ |
| Rₜ (HPLC) | 0.55 min (X012_S01) |

The following examples are prepared using procedures analogous to those described for example 78, using appropriate starting materials. As is appreciated by those skilled in the art, the analogous examples may involve variations in general reaction conditions.

| | | | HPLC retention time |
|---|---|---|---|
| Example | Structure | ESI-MS | [min] (method) |
| 79 | 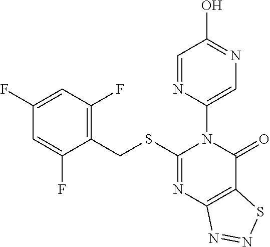 [SMILES: O=c1c2snnc2nc(SCc2c(F)cc(F)cc2F)n1-c1cnc(O)cn1] | 425 [M + H]⁺ | 0.53 (X012_S01) |
| 80 | 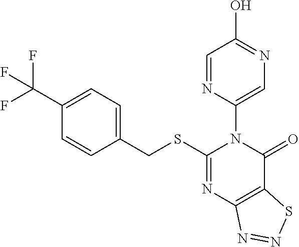 [SMILES: O=c1c2snnc2nc(SCc2ccc(C(F)(F)F)cc2)n1-c1cnc(O)cn1] | 439 [M + H]⁺ | 0.59 (X012_S01) |
| 81 | 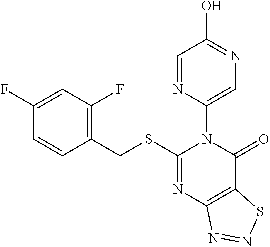 [SMILES: O=c1c2snnc2nc(SCc2ccc(F)cc2F)n1-c1cnc(O)cn1] | 407 [M + H]⁺ | 0.52 (X012_S01) |
| 82 | 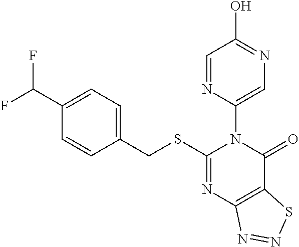 [SMILES: O=c1c2snnc2nc(SCc2ccc(C(F)F)cc2)n1-c1cnc(O)cn1] | 421 [M + H]⁺ | 0.57 (X012_S01) |

### Example 83

### 5-((4-Chlorobenzyl)thio)-6-(5-fluoro-6-hydroxypyridin-3-yl)-[1,2,3]thiadiazolo[4,5-d]pyrimidin-7(6H)-one

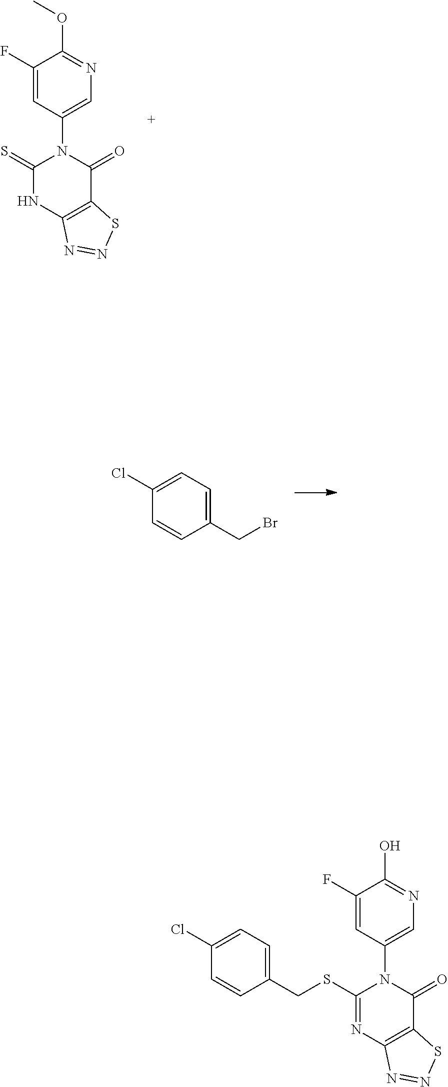 [SMILES: COc1ncc(-n2c(=S)[nH]c3nnsc3c2=O)cc1F ; Clc1ccc(CBr)cc1 ; O=c1c2snnc2nc(SCc2ccc(Cl)cc2)n1-c1cnc(O)c(F)c1]

1-(Bromomethyl)-4-chlorobenzene (98.5 mg, 470 μmol, 1.10 equiv) is added to a mixture of 6-(5-fluoro-6-methoxypyridin-3-yl)-5-thioxo-5,6-dihydro-[1,2,3]thiadiazolo[4,5-d]pyrimidin-7(4H)-one (intermediate 38, 133 mg, 427 μmol, 1 equiv), and triethylamine (179 μL, 1.28 mmol, 3.00 equiv) in N,N-dimethylacetamide (2 mL). After 3 h, ethyl acetate is added, and the mixture is washed with water. The organic layer is dried over magnesium sulfate, filtered, and concentrated under reduced pressure. The residue is dissolved in acetonitrile (10 mL) and treated with chlorotrimethylsilane (162 μL, 1.28 mmol, 3.00 equiv) and sodium iodide (128 mg, 854 μmol, 2.00 equiv). After 2 h, the precipitate is filtered, washed with water, and dried to yield the desired product.

| | |
|---|---|
| C₁₆H₉ClFN₅O₂S₂ | (MW = 421.0 g/mol) |
| ESI | 422 [M + H]⁺ |
| Rₜ (HPLC) | 0.74 min (X012_S01) |

The following examples are prepared using procedures analogous to those described for example 83, using appropriate starting materials. As is appreciated by those skilled in the art, the analogous examples may involve variations in general reaction conditions.

| | | | HPLC retention |
|---|---|---|---|
| Example | Structure | ESI-MS | time [min] (method) |
| 84 | 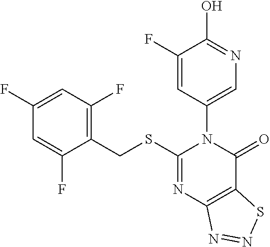 [SMILES: O=c1c2snnc2nc(SCc2c(F)cc(F)cc2F)n1-c1cnc(O)c(F)c1] | 442 [M + H]⁺ | 0.54 (X012_S01) |
| 85 | 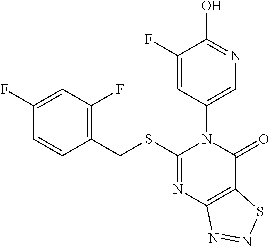 [SMILES: O=c1c2snnc2nc(SCc2ccc(F)cc2F)n1-c1cnc(O)c(F)c1] | 424 [M + H]⁺ | 0.53 (X012_S01) |
| 86 | 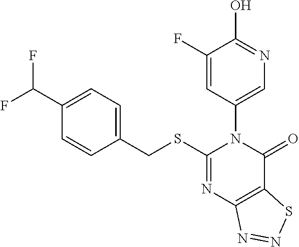 [SMILES: O=c1c2snnc2nc(SCc2ccc(C(F)F)cc2)n1-c1cnc(O)c(F)c1] | 438 [M + H]⁺ | 0.54 (X012_S01) |
| 87 | 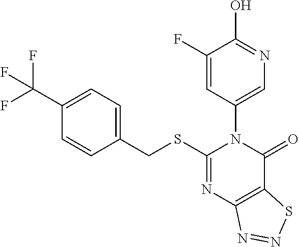 [SMILES: O=c1c2snnc2nc(SCc2ccc(C(F)(F)F)cc2)n1-c1cnc(O)c(F)c1] | 456 [M + H]⁺ | 0.59 (X012_S01) |

### Analytical HPLC Methods

| | |
|---|---|
| Method Name: | X011_S03 |
| Device description: | Waters Acquity with DA- and MS-Detector |
| Column: | XBridge BEH C18_2.1 × 30 mm_1.7 μm |
| Column producer: | Waters |
| Description: | |

| Gradient/ | % Sol | | | |
|---|---|---|---|---|
| Solvent Time | [Water | % Sol | Flow | Temp |
| [min] | 0.1% NH3] | [Acetonitrile] | [ml/min] | [° C.] |
| 0.0 | 95.0 | 5.0 | 1.3 | 60.0 |
| 0.02 | 95.0 | 5.0 | 1.3 | 60.0 |
| 1.0 | 0.0 | 100.0 | 1.3 | 60.0 |
| 1.1 | 0.0 | 100.0 | 1.3 | 60.0 |

| | |
|---|---|
| Method Name: | X011_S04 |
| Device description: | Waters Acquity with DA- and MS-Detector |
| Column: | XBridge BEH C18_2.1 × 30 mm_2.5 μm |
| Column producer: | Waters |
| Description: | |

| Gradient/ | % Sol | | | |
|---|---|---|---|---|
| Solvent Time | [Water | % Sol | Flow | Temp |
| [min] | 0.1% NH3] | [Acetonitrile] | [ml/min] | [° C.] |
| 0.0 | 95.0 | 5.0 | 1.3 | 60.0 |
| 0.02 | 95.0 | 5.0 | 1.3 | 60.0 |
| 1.0 | 0.0 | 100.0 | 1.3 | 60.0 |
| 1.1 | 0.0 | 100.0 | 1.3 | 60.0 |

| | |
|---|---|
| Method Name: | X011_S05 |
| Device description: | Waters Acquity with DA- and MS-Detector |
| Column: | XBridge BEH C18_2.1 × 30 mm_2.5 μm |
| Column producer: | Waters |
| Description: | |

| Gradient/ | % Sol | | | |
|---|---|---|---|---|
| Solvent Time | [Water | % Sol | Flow | Temp |
| [min] | 0.1% NH3] | [Acetonitrile] | [ml/min] | [° C.] |
| 0.0 | 95.0 | 5.0 | 1.3 | 60.0 |
| 0.02 | 95.0 | 5.0 | 1.3 | 60.0 |
| 1.0 | 0.0 | 100.0 | 1.3 | 60.0 |
| 1.3 | 0.0 | 100.0 | 1.3 | 60.0 |

| | |
|---|---|
| Method Name: | X012_S01 |
| Device description: | Waters Acquity with DA- and MS-Detector |
| Column: | XBridge BEH C18_2.1 × 30 mm_1.7 μm |
| Column producer: | Waters |
| Description: | |

| Gradient/ | % Sol | | | |
|---|---|---|---|---|
| Solvent Time | [Water 0.1% | % Sol | Flow | Temp |
| [min] | TFA (v/v)] | [Acetonitrile] | [ml/min] | [° C.] |
| 0.0 | 99.0 | 1.0 | 1.6 | 60.0 |
| 0.02 | 99.0 | 1.0 | 1.6 | 60.0 |
| 1.0 | 0.0 | 100.0 | 1.6 | 60.0 |
| 1.1 | 0.0 | 100.0 | 1.6 | 60.0 |

| | |
|---|---|
| Method Name: | X018_S03 |
| Device description: | Waters Acquity with DA- and MS-Detector |
| Column: | Sunfire C18_3.0 × 30 mm_2.5 μm |
| Column producer: | Waters |
| Description: | |

| Gradient/ | % Sol | | | |
|---|---|---|---|---|
| Solvent Time | [Water 0.1% | % Sol | Flow | Temp |
| [min] | TFA (v/v)] | [Acetonitrile] | [ml/min] | [° C.] |
| 0.0 | 95.0 | 5.0 | 1.5 | 60.0 |
| 1.3 | 0.0 | 100.0 | 1.5 | 60.0 |
| 1.5 | 0.0 | 100.0 | 1.5 | 60.0 |

| | |
|---|---|
| Method Name: | Z018_S04 |
| Device description: | Agilent 1200 with DA- and MS-Detector |
| Column: | Sunfire C18_3.0 × 30 mm_2.5 μm |
| Column producer: | Waters |
| Description: | |

| Gradient/ | % Sol | | | |
|---|---|---|---|---|
| Solvent Time | [Water 0.1% | % Sol | Flow | Temp |
| [min] | TFA (v/v)] | [Acetonitrile] | [ml/min] | [° C.] |
| 0.0 | 97.0 | 3.0 | 2.2 | 60.0 |
| 0.2 | 97.0 | 3.0 | 2.2 | 60.0 |
| 1.2 | 0.0 | 100.0 | 2.2 | 60.0 |
| 1.25 | 0.0 | 100.0 | 3.0 | 60.0 |
| 1.4 | 0.0 | 100.0 | 3.0 | 60.0 |

| | |
|---|---|
| Method Name: | Z020_S01 |
| Device description: | Agilent 1200 with DA- and MS-Detector |
| Column: | Sunfire C18_3.0 × 30 mm_2.5 μm |
| Column producer: | Waters |
| Description: | |

| Gradient/ | % Sol | | | |
|---|---|---|---|---|
| Solvent Time | [Water 0.1% | % Sol | Flow | Temp |
| [min] | FA (v/v)] | [Acetonitrile] | [ml/min] | [° C.] |
| 0.0 | 97.0 | 3.0 | 2.2 | 60.0 |
| 0.2 | 97.0 | 3.0 | 2.2 | 60.0 |
| 1.2 | 0.0 | 100.0 | 2.2 | 60.0 |
| 1.25 | 0.0 | 100.0 | 3.0 | 60.0 |
| 1.4 | 0.0 | 100.0 | 3.0 | 60.0 |

| | |
|---|---|
| Method Name: | 007_CA11 |
| Device description: | Waters Acquity, QDa Detector |
| Column: | Sunfire C18_3.0 × 30 mm_2.5 μm |
| Column producer: | Waters |
| Description: | |

| Gradient/ | % Sol | % Sol | | |
|---|---|---|---|---|
| Solvent Time | [Water 0.1% | [Acetonitrile | Flow | Temp |
| [min] | TFA (v/v)] | 0.08% TFA (v/v)] | [ml/min] | [° C.] |
| 0.0 | 95.0 | 5.0 | 1.5 | 60.0 |
| 1.3 | 0.0 | 100.0 | 1.5 | 60.0 |
| 1.5 | 0.0 | 100.0 | 1.5 | 60.0 |
| 1.6 | 95.0 | 5.0 | 1.5 | 60.0 |

| | |
|---|---|
| Method Name: | 007_CA02 |
| Device description: | Waters Acquity, QDa Detector |
| Column: | Sunfire C18_3.0 × 30 mm_2.5 μm |
| Column producer: | Waters |
| Description: | |

| Gradient/ | % Sol | % Sol | | |
|---|---|---|---|---|
| Solvent Time | [Water 0.1% | [Acetonitrile | Flow | Temp |
| [min] | TFA (v/v)] | 0.08% TFA (v/v)] | [ml/min] | [° C.] |
| 0.0 | 95.0 | 5.0 | 1.5 | 60.0 |
| 1.3 | 0.0 | 100.0 | 1.5 | 60.0 |
| 1.5 | 0.0 | 100.0 | 1.5 | 60.0 |
| 1.6 | 95.0 | 5.0 | 1.5 | 60.0 |

| | |
|---|---|
| Method Name: | 008_CA02 |
| Device description: | Waters Acquity, QDa Detector |
| Column: | XBridge C18_3.0 × 30 mm_2.5 μm |
| Column producer: | Waters |
| Description: | |

| Gradient/ | % Sol | | | |
|---|---|---|---|---|
| Solvent Time | [Water | % Sol | Flow | Temp |
| [min] | 0.1% NH3] | [Acetonitrile] | [ml/min] | [° C.] |
| 0.0 | 95.0 | 5.0 | 1.5 | 60.0 |
| 1.3 | 0.0 | 100.0 | 1.5 | 60.0 |
| 1.5 | 0.0 | 100.0 | 1.5 | 60.0 |
| 1.6 | 95.0 | 5.0 | 1.5 | 60.0 |

The invention encompasses the following preferred embodiments:

### PREFERRED EMBODIMENTS

1. A compound of formula (I)

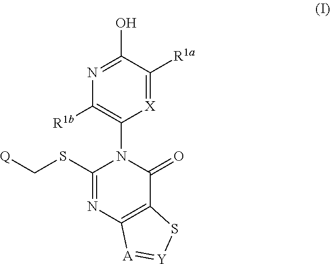

wherein
A is N or CH;
X is N or CR^{1c};
Y is N or CH;
R^{1a}, R^{1b} and R^{1c} is H or F;
Q is selected from among a group consisting of

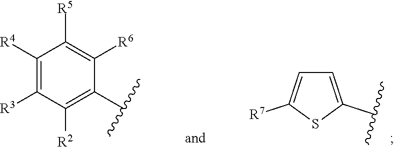

R², R³, R⁴, R⁵, R⁶ are, independently of each other, selected from among a group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃;
R⁷ is —Cl or —CF₃;
or a salt thereof;
provided that the compound is not

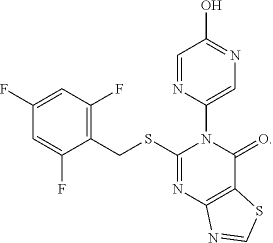 [SMILES: O=c1c2scnc2nc(SCc2c(F)cc(F)cc2F)n1-c1cnc(O)cn1]

2. A compound according to embodiment 1, wherein A is CH, Y is CH, R^{1c} is H and Q is

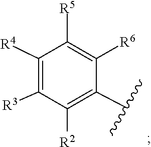

or a salt thereof.

3. A compound according to embodiment 1, wherein A is N, Y is CH and R^{1c} is H; or a salt thereof.

4. A compound according to embodiment 1, wherein A is N, Y is N, R^{1c} is H and Q is

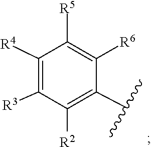

or a salt thereof.

5. A compound according to embodiment 3, wherein
Q is

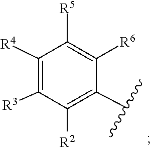

or a salt thereof.

6. A compound according to embodiment 3, wherein
Q is

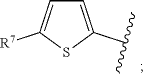

or a salt thereof.

7. A compound according to any one of embodiments 3, 4, 5 or 6 wherein X is N; or a salt thereof.

8. A compound according to any one of embodiments 3, 4, 5 or 6 wherein X is CH; or a salt thereof.

9. A compound according to embodiment 1 selected from among the group consisting of

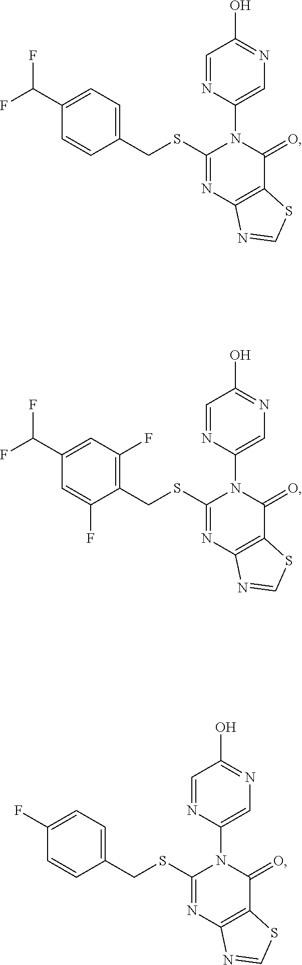 [SMILES: O=c1c2scnc2nc(SCc2ccc(C(F)F)cc2)n1-c1cnc(O)cn1 ; O=c1c2scnc2nc(SCc2c(F)cc(C(F)F)cc2F)n1-c1cnc(O)cn1 ; O=c1c2scnc2nc(SCc2ccc(F)cc2)n1-c1cnc(O)cn1]

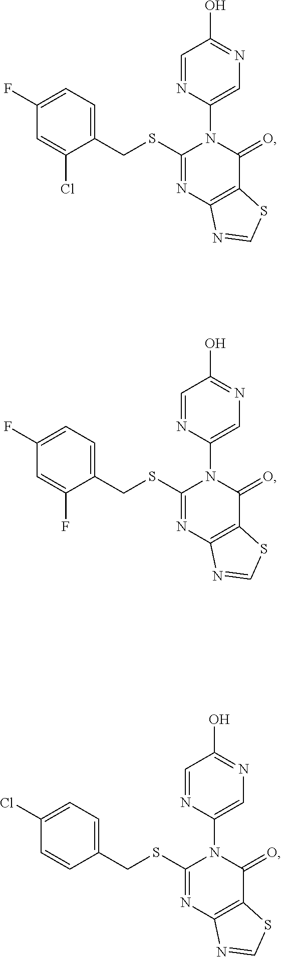 [SMILES: O=c1c2scnc2nc(SCc2ccc(F)cc2Cl)n1-c1cnc(O)cn1 ; O=c1c2scnc2nc(SCc2ccc(F)cc2F)n1-c1cnc(O)cn1 ; O=c1c2scnc2nc(SCc2ccc(Cl)cc2)n1-c1cnc(O)cn1]

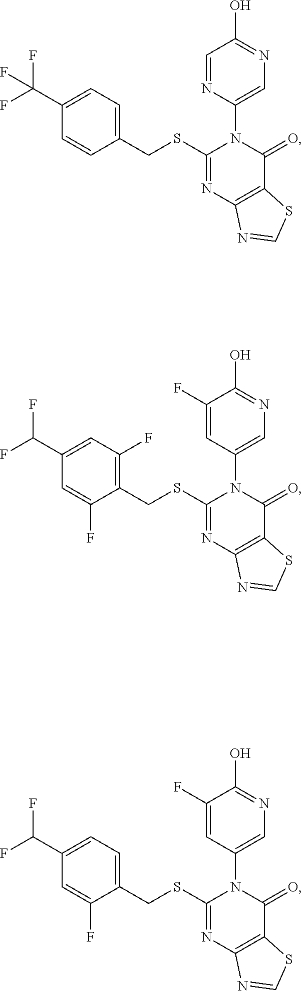 [SMILES: O=c1c2scnc2nc(SCc2ccc(C(F)(F)F)cc2)n1-c1cnc(O)cn1 ; O=c1c2scnc2nc(SCc2c(F)cc(C(F)F)cc2F)n1-c1cnc(O)c(F)c1 ; O=c1c2scnc2nc(SCc2ccc(C(F)F)cc2F)n1-c1cnc(O)c(F)c1]

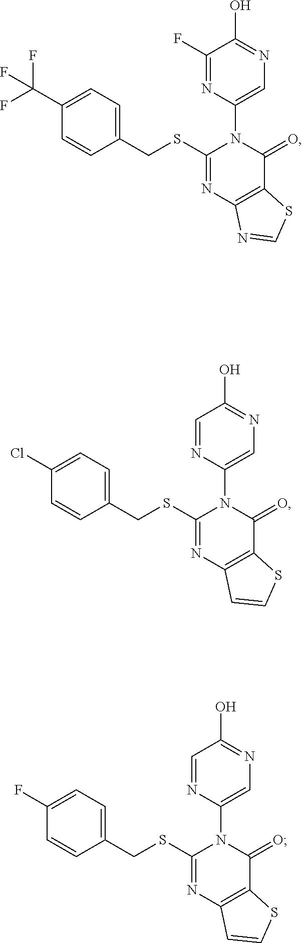 [SMILES: O=c1c2scnc2nc(SCc2ccc(C(F)(F)F)cc2)n1-c1cnc(O)c(F)n1 ; O=c1c2sccc2nc(SCc2ccc(Cl)cc2)n1-c1cnc(O)cn1 ; O=c1c2sccc2nc(SCc2ccc(F)cc2)n1-c1cnc(O)cn1]

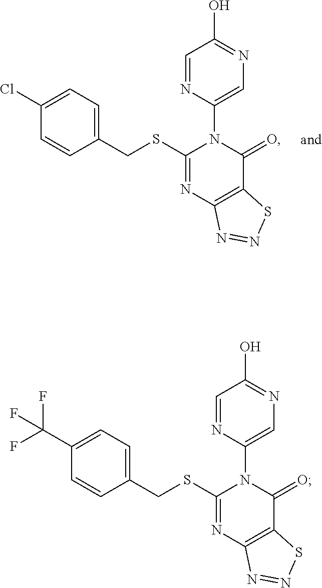 [SMILES: O=c1c2snnc2nc(SCc2ccc(Cl)cc2)n1-c1cnc(O)cn1 ; O=c1c2snnc2nc(SCc2ccc(C(F)(F)F)cc2)n1-c1cnc(O)cn1]

or a pharmaceutically acceptable salt thereof.

10. A compound according to any one of the embodiments 1-9 in its salt free form.

11. A compound according to any one of embodiments 1-9 or a pharmaceutically acceptable salt thereof for use as a medicament.

12. A compound according to any one of embodiments 1 to 9 or a pharmaceutically acceptable salt thereof for use in the treatment and/or prevention of a disease selected from among the group consisting of chronic liver diseases, portal hypertension, viral infections, cancer, interstitial lung diseases, retinopathies, acute and chronic inflammation as well as fibrotic diseases. interstitial lung diseases.

13. A compound according to any one of embodiments 1 to 9 or a pharmaceutically acceptable salt thereof for use in the treatment of and/or prevention of a disease selected from among the group consisting of vascular inflammation, atherosclerosis, interstitial lung diseases (e.g. idiopathic pulmonary fibrosis, progressive pulmonary fibrosis), liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors (pancreatic, lung, breast, colon, head and neck tumors), systemic sclerosis, inflammatory bowel disease, Duchenne muscular dystrophy, COVID-19, acute respiratory distress syndrome, influenza, ischemic and hemorrhagic stroke.

14. A pharmaceutical composition comprising at least one compound according to any one of embodiments 1 to 9 or a pharmaceutically acceptable salt thereof and a pharmaceutically acceptable carrier.

15. A medicament combination which comprises, besides one or more compounds according to one or more of embodiments 1 to 9, or a pharmaceutically acceptable salt thereof as further active substances a substance selected from the group consisting of antifibrotics, immunotherapeutics, sGC activators, ATX-inhibitors, SGLT2-inhibitors, THRb inhibitors, GLP1 agonists and GLP1 agonist combinations, FGF-analogs, KRAS-G12C-inhibitors, KRAS-G12D-inhibitors, MDM2-p53-antagonists, of Her2-inhibitors and chemotherapeutics.

16. A compound of formula (I)

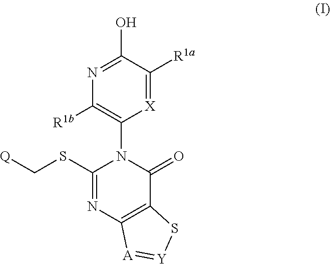

wherein
A is N or CH;
X is N or CR^{1c};
Y is N or CH;
R^{1a}, R^{1b} and R^{1c} is H or F;
Q is

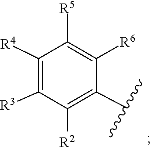

R², R³, R⁴, R⁵, R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃;
or a salt thereof.

17. A compound of formula (I)

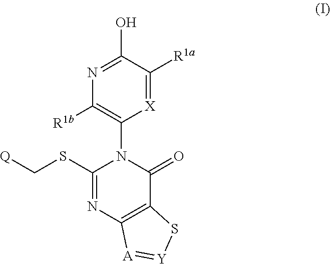

wherein
A is N or CH;
X is N or CR^{1c};
Y is N or CH;
R^{1a}, R^{1b} and R^{1c} is H or F;
Q is

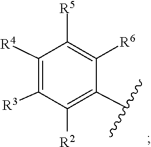

R², R³, R⁴, R⁵, R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃;
or a salt thereof;
provided that the compound is not

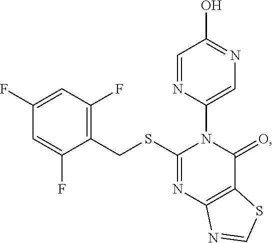 [SMILES: O=c1c2scnc2nc(SCc2c(F)cc(F)cc2F)n1-c1cnc(O)cn1]

and
provided that when A is N and Y is CH, X is not CR^{1c}.

1. A compound according to formula (Ib) 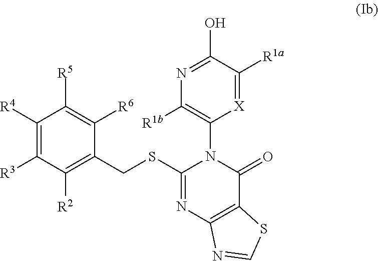
wherein
X is N;
R^{1a} and R^{1b} are, independently of each other, H or F;
R², R³, R⁴, R⁵, and R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

2. The compound according to claim 1, wherein
R^{1a} and R^{1b} are, independently of each other, H or F, provided that R^{1a} and R^{1b} cannot be F simultaneously;
R², R³, R⁴, R⁵, and R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, and R⁶ are H; or
a salt thereof.

3. The compound according to claim 1, wherein
R^{1a} and R^{1b} are, independently of each other, H or F, provided that R^{1a} and R^{1b} cannot be F simultaneously;
R², R⁴, and R⁶ are, independently of each other, selected from the group consisting of —H, —F, —Cl, —C₁₋₂-alkyl, cyclopropyl, —CH═CH₂, —C≡CH, —CF₃, —CF₂H, —CF₂Cl and —OCH₃;
R³ is H; and R⁵ is H; or a salt thereof.

4. The compound according to claim 1, wherein the compound is selected from the group consisting of 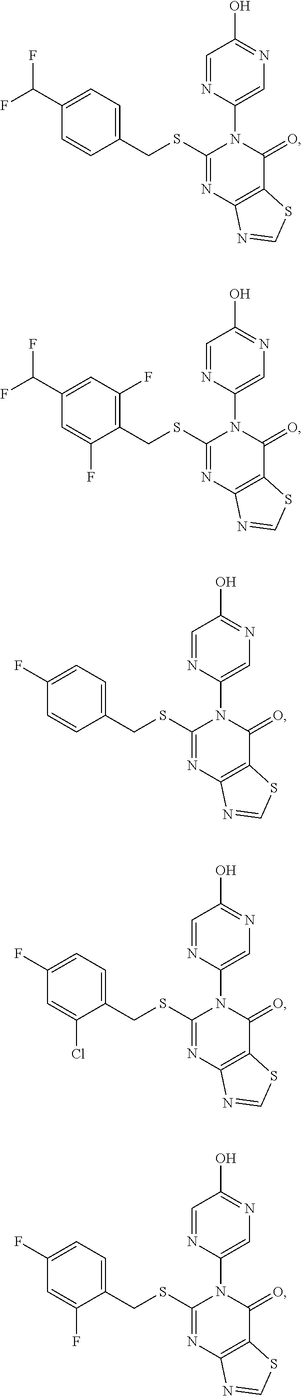 [SMILES: O=c1c2scnc2nc(SCc2ccc(C(F)F)cc2)n1-c1cnc(O)cn1 ; O=c1c2scnc2nc(SCc2c(F)cc(C(F)F)cc2F)n1-c1cnc(O)cn1 ; O=c1c2scnc2nc(SCc2ccc(F)cc2)n1-c1cnc(O)cn1 ; O=c1c2scnc2nc(SCc2ccc(F)cc2Cl)n1-c1cnc(O)cn1 ; O=c1c2scnc2nc(SCc2ccc(F)cc2F)n1-c1cnc(O)cn1] 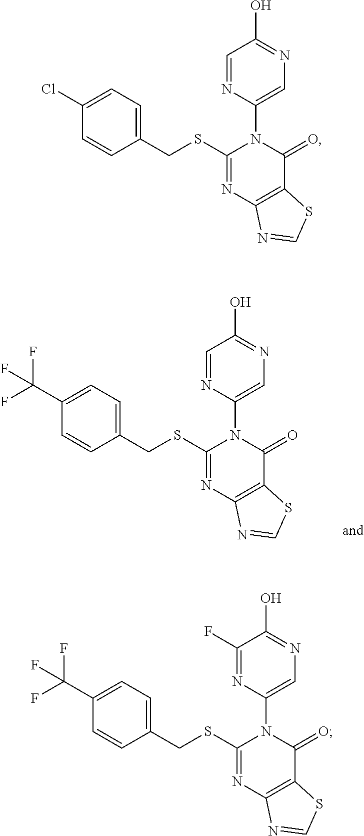 [SMILES: O=c1c2scnc2nc(SCc2ccc(Cl)cc2)n1-c1cnc(O)cn1 ; O=c1c2scnc2nc(SCc2ccc(C(F)(F)F)cc2)n1-c1cnc(O)cn1 ; O=c1c2scnc2nc(SCc2ccc(C(F)(F)F)cc2)n1-c1cnc(O)c(F)n1]
or a pharmaceutically acceptable salt thereof.

5. A pharmaceutical composition comprising at least one compound according to claim 1, or a pharmaceutically acceptable salt thereof, and a pharmaceutically acceptable carrier.

6. A pharmaceutical composition according to claim 5, further comprising an additional therapeutic agent selected from the group consisting of antifibrotics, immunotherapeutics, sGC activators, ATX-inhibitors, SGLT2-inhibitors, THRb inhibitors, GLP1 agonists and GLP1 agonist combinations, FGF-analogs, KRAS-G12C-inhibitors, KRAS-G12D-inhibitors, MDM2-p53-antagonists, of Her2-inhibitors and chemotherapeutics.

7. A method for the treatment and/or prevention of a disease selected from the group consisting of chronic liver diseases, portal hypertension, viral infections, cancer, interstitial lung diseases, retinopathies, acute and chronic inflammation and fibrotic diseases, said method comprising administering an effective amount of a compound according to claim 1, or a pharmaceutically acceptable salt thereof, to a human being.

8. The method according to claim 7 wherein the disease is selected in the group consisting of vascular inflammation, atherosclerosis, interstitial lung diseases, idiopathic pulmonary fibrosis, progressive pulmonary fibrosis, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors, NOX4+ cancer associated fibroblast rich tumors wherein the tumor is selected in the group consisting of pancreatic, lung, breast, colon, head and neck tumors, systemic sclerosis, inflammatory bowel disease, Duchenne muscular dystrophy, COVID-19, acute respiratory distress syndrome, influenza, ischemic and hemorrhagic stroke, heart failure, cardio myopathies, hypertension, progression of interstitial lung diseases, chronic lung disease, chronic obstructive pulmonary disease (COPD), asthma, diabetic nephropathy, acute kidney injury, obstructive nephropathy, hypertensive nephropathy, and neurodegenerative diseases.

9. A compound according to formula (Ia) 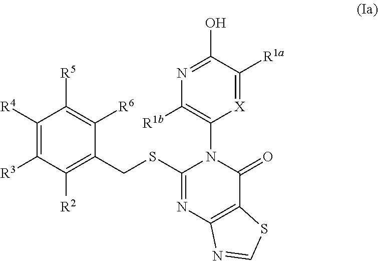
wherein
X is N or CR^{1c};
R^{1c} is H;
R^{1a} and R^{1b} are, independently of each other, H or F;
R², R³, R⁴, R⁵, and R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

10. The compound according to claim 9, wherein
R^{1a} and R^{1b} are, independently of each other, H or F, provided that R^{1a} and R^{1b} cannot be F simultaneously;
R², R³, R⁴, R⁵, and R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, and R⁶ are H; or
a salt thereof.

11. The compound according to claim 9, wherein
R^{1a} and R^{1b} are, independently of each other, H or F, provided that R^{1a} and R^{1b} cannot be F simultaneously;
R², R⁴, and R⁶ are, independently of each other, selected from the group consisting of —H, —F, —Cl, —C₁₋₂-alkyl, cyclopropyl, —CH═CH₂, —C≡CH, —CF₃, —CF₂H, —CF₂Cl and —OCH₃;
R³ is H; and R⁵ is H; or a salt thereof.

12. A pharmaceutical composition comprising at least one compound according to claim 9, or a pharmaceutically acceptable salt thereof, and a pharmaceutically acceptable carrier.

13. A pharmaceutical composition according to claim 12, further comprising an additional therapeutic agent selected from the group consisting of antifibrotics, immunotherapeutics, sGC activators, ATX-inhibitors, SGLT2-inhibitors, THRb inhibitors, GLP1 agonists and GLP1 agonist combinations, FGF-analogs, KRAS-G12C-inhibitors, KRASG12D-inhibitors, MDM2-p53-antagonists, of Her2-inhibitors and chemotherapeutics.

14. A method for the treatment and/or prevention of a disease selected from the group consisting of chronic liver diseases, portal hypertension, viral infections, cancer, interstitial lung diseases, retinopathies, acute and chronic inflammation and fibrotic diseases, said method comprising administering an effective amount of a compound according to claim 9, or a pharmaceutically acceptable salt thereof, to a human being.

15. The method according to claim 14 wherein the disease is selected in the group consisting of vascular inflammation, atherosclerosis, interstitial lung diseases, idiopathic pulmonary fibrosis, progressive pulmonary fibrosis, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors, NOX4+ cancer associated fibroblast rich tumors wherein the tumor is selected in the group consisting of pancreatic, lung, breast, colon, head and neck tumors, systemic sclerosis, inflammatory bowel disease, Duchenne muscular dystrophy, COVID-19, acute respiratory distress syndrome, influenza, ischemic and hemorrhagic stroke, heart failure, cardio myopathies, hypertension, progression of interstitial lung diseases, chronic lung disease, chronic obstructive pulmonary disease (COPD), asthma, diabetic nephropathy, acute kidney injury, obstructive nephropathy, hypertensive nephropathy, and neurodegenerative diseases.

16. A compound according to formula (Id) 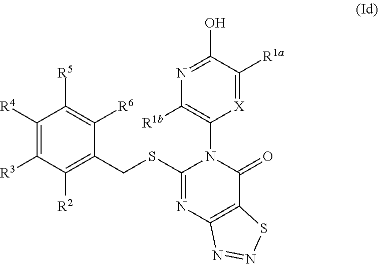
wherein
X is N or CR^{1c};
R^{1c} is H;
R^{1a} and R^{1b} are, independently of each other, H or F;
R², R³, R⁴, R⁵, and R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; or a salt thereof.

17. The compound according to claim 16, wherein
R^{1a} and R^{1b} are, independently of each other, H or F, provided that R^{1a} and R^{1b} cannot be F simultaneously;
R², R³, R⁴, R⁵, and R⁶ are, independently of each other, selected from the group consisting of —H, -halogen, —C₁₋₄-alkyl, cyclopropyl, —C₂₋₄-alken-1-yl, —C₂₋₄-alkyn-1-yl, —C₁₋₄-haloalkyl, —O—C₁₋₄-alkyl, —CN, —SF₅ and —N₃; provided that at least two of R², R³, R⁴, R⁵, and R⁶ are H; or
a salt thereof.

18. The compound according to claim 16, wherein
R^{1a} and R^{1b} are, independently of each other, H or F, provided that R^{1a} and R^{1b} cannot be F simultaneously;
R², R⁴, and R⁶ are, independently of each other, selected from the group consisting of —H, —F, —Cl, —C₁₋₂-alkyl, cyclopropyl, —CH═CH₂, —C≡CH, —CF₃, —CF₂H, —CF₂Cl and —OCH₃;
R³ is H; and R⁵ is H; or a salt thereof.

19. A pharmaceutical composition comprising at least one compound according to claim 16, or a pharmaceutically acceptable salt thereof, and a pharmaceutically acceptable carrier.

20. A pharmaceutical composition according to claim 19, further comprising an additional therapeutic agent selected from the group consisting of antifibrotics, immunotherapeutics, sGC activators, ATX-inhibitors, SGLT2-inhibitors, THRb inhibitors, GLP1 agonists and GLP1 agonist combinations, FGF-analogs, KRAS-G12C-inhibitors, KRASG12D-inhibitors, MDM2-p53-antagonists, of Her2-inhibitors and chemotherapeutics.

21. A method for the treatment and/or prevention of a disease selected from the group consisting of chronic liver diseases, portal hypertension, viral infections, cancer, interstitial lung diseases, retinopathies, acute and chronic inflammation and fibrotic diseases, said method comprising administering an effective amount of a compound according to claim 16, or a pharmaceutically acceptable salt thereof, to a human being.

22. The method according to claim 21 wherein the disease is selected in the group consisting of vascular inflammation, atherosclerosis, interstitial lung diseases, idiopathic pulmonary fibrosis, progressive pulmonary fibrosis, liver fibrosis, pulmonary hypertension, portal hypertension, liver cirrhosis, acute on chronic liver failure (ACLF), sepsis, multi-organ failure, diabetic retinopathies, wet age-related macular degeneration (AMD), dry AMD, cardiovascular diseases, NOX4+ cancer associated fibroblast rich tumors, NOX4+ cancer associated fibroblast rich tumors wherein the tumor is selected in the group consisting of pancreatic, lung, breast, colon, head and neck tumors, systemic sclerosis, inflammatory bowel disease, Duchenne muscular dystrophy, COVID-19, acute respiratory distress syndrome, influenza, ischemic and hemorrhagic stroke, heart failure, cardio myopathies, hypertension, progression of interstitial lung diseases, chronic lung disease, chronic obstructive pulmonary disease (COPD), asthma, diabetic nephropathy, acute kidney injury, obstructive nephropathy, hypertensive nephropathy, and neurodegenerative diseases.
